(19) United States
(12) Patent Application Publication
(10) Pub. No.: US 2024/0368653 A1
(43) Pub. Date: Nov. 7, 2024

(54) MODIFIED ß-GALACTOSIDASE

(71) Applicant: AMANO ENZYME INC., Nagoya-shi (JP)

(72) Inventors: Genichi Nakamura, Kakamigahara-shi (JP); Yuko Kuritani, Kakamigahara-shi (JP); Satour Ishihara, Kakamigahara-shi (JP)

(73) Assignee: AMANO ENZYME INC., Nagoya-shi (JP)

(21) Appl. No.: 18/776,771

(22) Filed: Jul. 18, 2024

Related U.S. Application Data
(62) Division of application No. 18/080,926, filed on Dec. 14, 2022.
Application No. 18/080,926 is a division of application No. 16/574,149, filed on Sep. 18, 2019, now Pat. No. 11,555,214.
Application No. 16/574,149 is a division of application No. 15/022,685, filed on May 5, 2016, now Pat. No. 10,465,219.

(30) Foreign Application Priority Data
Sep. 30, 2013 (JP) 2013-205097

(51) Int. Cl. C12P 19/14; C12N 9/38; C12P 19/04; C12P 19/12

(52) U.S. Cl. CPC C12P 19/14; C12N 9/2471; C12P 19/04; C12P 19/12; C12Y 302/01023

(57) ABSTRACT

The usefulness of β-galactosidases derived from Bacillus circulans is further enhanced. A modified β-galactosidase in which one or more amino acids selected from the group consisting of proline 182 (P182), tyrosine 187 (Y187), serine 188 (S188), tryptophan 405 (W405), alanine 406 (A406), glutamine 407 (Q407), tyrosine 449 (Y449), threonine 483 (T483), serine 512 (S512), serine 531 (S531), threonine 533 (T533), serine 534 (S534), asparagine 550 (N550), glutamine 551 (Q551), tryptophan 593 (W593), tyrosine 598 (Y598), proline 602 (P602), proline 604 (P604), tyrosine 609 (Y609), lysine 612 (K612), and tyrosine 615 (Y615), or an amino acid(s) corresponding thereto, has/have been substituted by other amino acid in a β-galactosidase consisting of the amino acid sequence of any of SEQ ID NOs. 1 to 4 or an amino acid sequence having 90% or more identity to the amino acid sequence set forth in any of SEQ ID NOs. 1 to 4.

### TECHNICAL FIELD

The present invention relates to a β-galactosidase. Specifically, the present invention relates to modifications of a β-galactosidase derived from Bacillus circulans, uses of modified enzymes thereof, and others. The present application claims priority to Japanese Patent Application No. 2013-205097, filed on Sep. 30, 2013, the entire content of which is incorporated herein by reference.

### SEQUENCE LISTING

The instant application contains a Sequence Listing which has been submitted in XML format and is hereby incorporated by reference in its entirety. The XML Sequence Listing file is named 18080926_SEQLIST.xml and was created on May 15, 2023 and is 28,952 bytes in size.

### BACKGROUND ART

β-galactosidase (EC 3.2.1.23) is an enzyme which hydrolyzes a β-D-galactoside linkage to release D-galactose, and β-galactosidase isozymes are generally found in a wide range of microorganisms and plants. β-galactosidase is otherwise referred to as lactase. β-galactosidase is also capable of transferring a galactoside linkage, and there are known methods in which this capability is used to produce galacto-oligosaccharides (oligosaccharides having galactose residues).

Galacto-oligosaccharides, which are linear or branched polymers of galactose, are composed of galactose except that they terminate in a glucose unit. Galacto-oligosaccharides are contained in mother's milk, which is reported to be a mixture of a variety of galacto-oligosaccharides (Non Patent Literature 1).

### CITATIONS LIST

### Patent Literature

Patent Literature 1: WO 2010/140435

### Non Patent Literatures

Non Patent Literature 1: Urashima et al., KAGAKU TO SEIBUTSU [Chemistry and Biology], 50, 7 (2012)
Non Patent Literature 2: Song et al., Biosci. Biotechnol. Biochem., 77, 73-79 (2013)

### SUMMARY OF INVENTION

### Technical Problems

Various β-galactosidases are known to be produced by a koji mold Aspergillus oryzae, yeasts Kluyveromyces lactis and Kluyveromyces marxinus, a bacterium Bacillus circulans, and others. Among β-galactosidase enzymes produced by these microorganisms, the β-galactosidase derived from Bacillus circulans, which is a mixture of β-galactosidase isozymes, is an enzyme allowing the production of galacto-oligosaccharides from lactose, and is an important enzyme in the industrial production of galacto-oligosaccharides (for example, a preparation of β-galactosidase isozymes is marketed under a trade name of “BIOLACTA”). The galacto-oligosaccharides produced using this enzyme preparation are highly uniform in composition, and it has been difficult that the composition (profile) of the galacto-oligosaccharides to be produced is altered even though modifications of production conditions and others have been made. Thus, the present invention mainly aims at providing a modified β-galactosidase of which the enzymatic specificity has been modified as desired in order to further enhance the usefulness of the β-galactosidase derived from Bacillus circulans and make it possible to that the enzyme is applied to new applications, for example, production of oligosaccharides which cannot be produced or are produced with low efficiency by the wild-type enzyme.

### Solutions to Problems

In previous studies, it was difficult to obtain the steric structure information of β-galactosidase isozymes derived from Bacillus circulans, so that it has not been easy to identify amino acids responsible for functions of these enzymes. For this reason, the inventors attempted to use computer simulation for prediction of their steric structures, and at the same time, to select amino acid residues useful in altering their properties, taking note of characteristic domain structures of the enzymes. Various variant enzymes were designed by introducing mutations at the positions of selected amino acid residues and examined for their properties, with the result that variants with apparently altered enzymatic specificities were found, for example, ones with decreased or increased hydrolytic activities for a β-1,6-bond. Accordingly, the inventors were able to identify amino acid residues that are useful and important in modifying the enzymatic specificity and successfully obtained a plurality of variants (modified enzymes) having altered enzymatic specificities. In addition, variants were designed taking note of a previous report (Non Patent Literature 2) and investigated in detail for their properties, with the result that other amino acids residues were also found which were useful in altering enzymatic specificity.

As is often experienced, on the other hand, combinations of two amino acid substitutions that are effective are highly likely to bring about additive or synergistic effects. Therefore, it can be said that the positions for mutation that have been successfully identified are effective, not only each alone, but also in combination thereof, in altering enzymatic properties.

On the basis of the above-described results and speculations, the following inventions are provided:

[1] A modified β-galactosidase consisting of an amino acid sequence in which one or more amino acids selected from the group consisting of proline at position 182 (P182), tyrosine at position 187 (Y187), serine at position 188 (S188), tryptophan at position 405 (W405), alanine at position 406 (A406), glutamine at position 407 (Q407), tyrosine at position 449 (Y449), threonine at position 483 (T483), serine at position 512 (S512), serine at position 531 (S531), threonine at position 533 (T533), serine at position 534 (S534), asparagine at position 550 (N550), glutamine at position 551 (Q551), tryptophan at position 593 (W593), tyrosine at position 598 (Y598), proline at position 602 (P602), proline at position 604 (P604), tyrosine at position 609 (Y609), lysine at position 612 (K612), and tyrosine at position 615 (Y615), or an amino acid(s) corresponding thereto, has/have been substituted by other amino acid in a β-galactosidase consisting of the amino acid sequence of any of SEQ ID NOs. 1 to 4 or an amino acid sequence having 90% or more identity to the amino acid sequence set forth in any of SEQ ID NOs. 1 to 4.

[2] The modified β-galactosidase according to [1], wherein the one or more amino acids to be substituted are selected from the group consisting of tyrosine at position 449 (Y449), serine at position 531 (S531), tryptophan at position 593 (W593), proline at position 602 (P602), proline at position 604 (P604), and lysine at position 612 (K612).

[3] The modified β-galactosidase according to [1], wherein the one or more amino acids to be substituted are selected from the group consisting of tyrosine at position 187 (Y187), serine at position 188 (S188), threonine at position 533 (T533), proline at position 602 (P602), and lysine at position 612 (K612).

[4] The modified β-galactosidase according to [1], wherein the one or more amino acids to be substituted are selected from the group consisting of proline at position 182 (P182), tyrosine at position 187 (Y187), serine at position 188 (S188), tryptophan at position 405 (W405), glutamine at position 407 (Q407), tyrosine at position 449 (Y449), threonine at position 483 (T483), serine at position 512 (S512), serine at position 531 (S531), asparagine at position 550 (N550), proline at position 602 (P602), and proline at position 604 (P604).

[5] The modified β-galactosidase according to [1], wherein the one or more amino acids to be substituted are selected from the group consisting of threonine at position 483 (T483), serine at position 512 (S512), glutamine at position 551 (Q551), tyrosine at position 598 (Y598), and tyrosine at position 615 (Y615).

[6] The modified β-galactosidase according to [1], wherein the one or more amino acids to be substituted are selected from the group consisting of tryptophan at position 405 (W405), threonine at position 483 (T483), serine at position 531 (S531), asparagine at position 550 (N550), and tyrosine at position 598 (Y598).

[7] The modified β-galactosidase according to [1], wherein the one or more amino acids to be substituted are selected from the group consisting of proline at position 182 (P182), tyrosine at position 187 (Y187), serine at position 188 (S188), tryptophan at position 405 (W405), alanine at position 406 (A406), glutamine at position 407 (Q407), tyrosine at position 449 (Y449), threonine at position 483 (T483), serine at position 512 (S512), serine at position 534 (S534), asparagine at position 550 (N550), glutamine at position 551 (Q551), proline at position 602 (P602), tyrosine at position 609 (Y609), lysine at position 612 (K612), and tyrosine at position 615 (Y615).

[8] The modified β-galactosidase according to [2], wherein the amino acid that has been substituted for tyrosine at position 449 (Y449) is aspartic acid, glutamic acid, asparagine, or tryptophan; the amino acid that has been substituted for serine at position 531 (S531) is alanine, aspartic acid, glutamic acid, phenylalanine, glycine, isoleucine, methionine, asparagine, or glutamine; the amino acid that has been substituted for tryptophan at position 593 (W593) is phenylalanine; the amino acid that has been substituted for proline at position 602 (P602) is glutamic acid or glycine; the amino acid that has been substituted for proline at position 604 (P604) is alanine, glycine, histidine, isoleucine, methionine, serine, threonine, valine, tryptophan, or tyrosine; and the amino acid that has been substituted for lysine at position 612 (K612) is methionine.

[9] The modified β-galactosidase according to [3], wherein the amino acid that has been substituted for tyrosine at position 187 (Y187) is aspartic acid, glutamic acid, or glycine; the amino acid that has been substituted for serine at position 188 (S188) is proline or threonine; the amino acid that has been substituted for threonine at position 533 (T533) is methionine or glutamine; the amino acid that has been substituted for proline at position 602 (P602) is cysteine, isoleucine, asparagine, serine, threonine, or valine; and the amino acid that has been substituted for lysine at position 612 (K612) is phenylalanine.

[10] The modified β-galactosidase according to [4], wherein the amino acid that has been substituted for proline at position 182 (P182) is leucine; the amino acid that has been substituted for tyrosine at position 187 (Y187) is aspartic acid or leucine; the amino acid that has been substituted for serine at position 188 (S188) is isoleucine; the amino acid that has been substituted for tryptophan at position 405 (W405) is alanine, aspartic acid, glutamic acid, glycine, histidine, leucine, asparagine, serine, or threonine; the amino acid that has been substituted for glutamine at position 407 (Q407) is glycine or arginine; the amino acid that has been substituted for tyrosine at position 449 (Y449) is glutamic acid, isoleucine, glutamine, arginine, or serine; the amino acid that has been substituted for threonine at position 483 (T483) is glutamine; the amino acid that has been substituted for serine at position 512 (S512) is phenylalanine, threonine, or valine; the amino acid that has been substituted for serine at position 531 (S531) is proline; the amino acid that has been substituted for asparagine at position 550 (N550) is leucine, arginine, or serine; the amino acid that has been substituted for proline at position 602 (P602) is leucine, asparagine, or glutamine; and the amino acid that has been substituted for proline at position 604 (P604) is isoleucine or methionine.

[11] The modified β-galactosidase according to [5], wherein the amino acid that has been substituted for threonine at position 483 (T483) is lysine, methionine, or arginine; the amino acid that has been substituted for serine at position 512 (S512) is glycine or lysine; the amino acid that has been substituted for glutamine at position 551 (Q551) is asparagine; the amino acid that has been substituted for tyrosine at position 598 (Y598) is asparagine; and the amino acid that has been substituted for tyrosine at position 615 (Y615) is threonine.

[12] The modified β-galactosidase according to [6], wherein the amino acid that has been substituted for tryptophan at position 405 (W405) is alanine, aspartic acid, serine, or threonine; the amino acid that has been substituted for threonine at position 483 (T483) is methionine; the amino acid that has been substituted for serine at position 531 (S531) is aspartic acid, phenylalanine, glycine, leucine, lysine, or methionine; the amino acid that has been substituted for asparagine at position 550 (N550) is phenylalanine; and the amino acid that has been substituted for tyrosine at position 598 (Y598) is phenylalanine or histidine.

[13] The modified β-galactosidase according to [7], wherein the amino acid that has been substituted for proline at position 182 (P182) is leucine or serine; the amino acid that has been substituted for tyrosine at position 187 (Y187) is aspartic acid, glutamic acid, or asparagine; the amino acid that has been substituted for serine at position 188 (S188) is glutamic acid, glycine, isoleucine, or asparagine; the amino acid that has been substituted for tryptophan at position 405 (W405) is histidine; the amino acid that has been substituted for alanine at position 406 (A406) is proline; the amino acid that has been substituted for glutamine at position 407 (Q407) is alanine, phenylalanine, glycine, lysine, or arginine; the amino acid that has been substituted for tyrosine at position 449 (Y449) is glutamic acid or arginine; the amino acid that has been substituted for threonine at position 483 (T483) is serine or glutamine; the amino acid that has been substituted for serine at position 512 (S512) is glycine, threonine, or valine; the amino acid that has been substituted for serine at position 534 (S534) is lysine or glutamine; the amino acid that has been substituted for asparagine at position 550 (N550) is alanine, glycine, lysine, leucine, methionine, arginine, or serine; the amino acid that has been substituted for glutamine at position 551 (Q551) is lysine or histidine; the amino acid that has been substituted for proline at position 602 (P602) is cysteine, aspartic acid, or asparagine; the amino acid that has been substituted for tyrosine at position 609 (Y609) is aspartic acid, glutamine, or threonine; the amino acid that has been substituted for lysine at position 612 (K612) is phenylalanine or isoleucine; and the amino acid that has been substituted for tyrosine at position 615 (Y615) is glutamic acid, isoleucine, or threonine.

[14] The modified β-galactosidase according to any one of [1] to [13], wherein the f-galactosidase consists of the amino acid sequence of SEQ ID NO. 1, and the modified β-galactosidase consists of an amino acid sequence in which in addition to the substitution, the tryptophan at position 1540 (W1540) is substituted with of the amino acid.

[15] A modified f-galactosidase consisting of an amino acid sequence in which one or more amino acids selected from the group consisting of tryptophan at position 85 (W85), phenylalanine at position 91 (F91), tyrosine at position 103 (Y103), tyrosine at position 134 (Y134), tyrosine at position 153 (Y153), proline at position 182 (P182), tryptophan at position 186 (W186), tyrosine at position 192 (Y192), tyrosine at position 295 (Y295), histidine at position 345 (H345), glycine at position 349 (G349), phenylalanine at position 402 (F402), glutamine at position 407 (Q407), glycine at position 479 (G479), glutamic acid at position 480 (E480), lysine at position 482 (K482), threonine at position 483 (T483), arginine at position 484 (R484), glycine at position 485 (G485), aspartic acid at position 486 (D486), lysine at position 487 (K487), valine at position 488 (V488), tyrosine at position 496 (Y496), serine at position 512 (S512), glutamic acid at position 513 (E513), asparagine at position 514 (N514), asparagine at position 515 (N515), tyrosine at position 516 (Y516), aspartic acid at position 517 (D517), tyrosine at position 519 (Y519), threonine at position 533 (T533), tyrosine at position 547 (Y547), tyrosine at position 549 (Y549), tyrosine at position 555 (Y555), arginine at position 572 (R572), threonine at position 573 (T573), tyrosine at position 598 (Y598), tyrosine at position 605 (Y605), tyrosine at position 606 (Y606), tyrosine at position 609 (Y609), phenylalanine at position 616 (F616), phenylalanine at position 624 (F624), tryptophan at position 648 (W648), and tryptophan at position 650 (W650), or an amino acid(s) corresponding thereto, has/have been substituted by other amino acid in a β-galactosidase consisting of the amino acid sequence of any of SEQ ID NOs. 1 to 4 or an amino acid sequence having 90% or more identity to the amino acid sequence set forth in any of SEQ ID NOs. 1 to 4.

[16] The modified β-galactosidase according to [15], wherein the amino acid that has been substituted for tryptophan at position 85 (W85) is glycine; the amino acid that has been substituted for phenylalanine at position 91 (F91) is alanine or isoleucine; the amino acid that has been substituted for tyrosine at position 103 (Y103) is glycine; the amino acid that has been substituted for tyrosine at position 134 (Y134) is glycine; the amino acid that has been substituted for tyrosine at position 153 (Y153) is glycine; the amino acid that has been substituted for proline at position 182 (P182) is tryptophan or tyrosine; the amino acid that has been substituted for tryptophan at position 186 (W186) is methionine; the amino acid that has been substituted for tyrosine at position 192 (Y192) is glycine; the amino acid that has been substituted for tyrosine at position 295 (Y295) is glycine; the amino acid that has been substituted for histidine at position 345 (H345) is glycine; the amino acid that has been substituted for glycine at position 349 (G349) is tryptophan; the amino acid that has been substituted for phenylalanine at position 402 (F402) is glycine; the amino acid that has been substituted for glutamine at position 407 (Q407) is isoleucine, leucine, methionine, valine, or tyrosine; the amino acid that has been substituted for glycine at position 479 (G479) is leucine, proline, or glutamine; the amino acid that has been substituted for glutamic acid at position 480 (E480) is asparagine; the amino acid that has been substituted for lysine at position 482 (K482) is methionine or valine; the amino acid that has been substituted for threonine at position 483 (T483) is glycine or serine; the amino acid that has been substituted for arginine at position 484 (R484) is phenylalanine, isoleucine, methionine, asparagine, or glutamine; the amino acid that has been substituted for glycine at position 485 (G485) is aspartic acid, asparagine, glutamine, or threonine; the amino acid that has been substituted for aspartic acid at position 486 (D486) is alanine, glutamic acid, phenylalanine, isoleucine, lysine, leucine, methionine, proline, glutamine, arginine, threonine, tryptophan, or tyrosine; the amino acid that has been substituted for lysine at position 487 (K487) is cysteine, glutamic acid, phenylalanine, histidine, isoleucine, leucine, asparagine, glutamine, arginine, threonine, valine, tryptophan, or tyrosine; the amino acid that has been substituted for valine at position 488 (V488) is glycine, isoleucine, leucine, arginine, serine, threonine, tryptophan, or tyrosine; the amino acid that has been substituted for tyrosine at position 496 (Y496) is glycine; the amino acid that has been substituted for serine at position 512 (S512) is cysteine, aspartic acid, glutamic acid, asparagine, proline, glutamine, or tryptophan; the amino acid that has been substituted for glutamic acid at position 513 (E513) is alanine, cysteine, aspartic acid, phenylalanine, glycine, histidine, lysine, leucine, methionine, asparagine, proline, glutamine, arginine, serine, threonine, or valine; the amino acid that has been substituted for asparagine at position 514 (N514) is glutamic acid, leucine, or lysine; the amino acid that has been substituted for asparagine at position 515 (N515) is alanine, aspartic acid, glutamic acid, glycine, isoleucine, leucine, proline, or valine; the amino acid that has been substituted for tyrosine at position 516 (Y516) is tryptophan; the amino acid that has been substituted for aspartic acid at position 517 (D517) is lysine or tryptophan; the amino acid that has been substituted for tyrosine at position 519 (Y519) is glycine; the amino acid that has been substituted for threonine at position 533 (T533) is glutamic acid; the amino acid that has been substituted for tyrosine at position 547 (Y547) is glycine; the amino acid that has been substituted for tyrosine at position 549 (Y549) is glycine; the amino acid that has been substituted for tyrosine at position 555 (Y555) is glycine; the amino acid that has been substituted for arginine at position 572 (R572) is cysteine, leucine, methionine, threonine, tryptophan, or tyrosine; the amino acid that has been substituted for threonine at position 573 (T573) is glutamic acid; the amino acid that has been substituted for tyrosine at position 598 (Y598) is histidine or asparagine; the amino acid that has been substituted for tyrosine at position 605 (Y605) is cysteine, aspartic acid, histidine, isoleucine, lysine, proline, arginine, or threonine; the amino acid that has been substituted for tyrosine at position 606 (Y606) is alanine, cysteine, aspartic acid, glutamic acid, phenylalanine, glycine, histidine, isoleucine, lysine, leucine, methionine, or serine; the amino acid that has been substituted for tyrosine at position 609 (Y609) is glutamic acid, asparagine, or serine; the amino acid that has been substituted for phenylalanine at position 616 (F616) is glycine; the amino acid that has been substituted for phenylalanine at position 624 (F624) is glycine; the amino acid that has been substituted for tryptophan at position 648 (W648) is glycine; and the amino acid that has been substituted for tryptophan at position 650 (W650) is glycine.

[17] A modified β-galactosidase consisting of an amino acid sequence in which one or more amino acids selected from the group consisting of tryptophan at position 76 (W76), glutamic acid at position 480 (E480), threonine at position 483 (T483), glycine at position 485 (G485), arginine at position 484 (R484), lysine at position 487 (K487), valine at position 488 (V488), serine at position 512 (S512), asparagine at position 514 (N514), asparagine at position 515 (N515), glycine at position 569 (G569), and phenylalanine at position 596 (F596), or an amino acid(s) corresponding thereto, has/have been substituted by other amino acid in a f-galactosidase consisting of the amino acid sequence of any of SEQ ID NOs. 1 to 4 or an amino acid sequence having 90% or more identity to the amino acid sequence set forth in any of SEQ ID NOs. 1 to 4.

[18] The modified β-galactosidase according to [17], wherein the amino acid that has been substituted for tryptophan at position 76 (W76) is glycine; the amino acid that has been substituted for glutamic acid at position 480 (E480) is glutamine; the amino acid that has been substituted for threonine at position 483 (T483) is tryptophan; the amino acid that has been substituted for glycine at position 485 (G485) is proline or threonine; the amino acid that has been substituted for arginine at position 484 (R484) is alanine, cysteine, aspartic acid, glycine, proline, serine, tyrosine, or lysine; the amino acid that has been substituted for lysine at position 487 (K487) is glycine or proline; the amino acid that has been substituted for valine at position 488 (V488) is proline; the amino acid that has been substituted for serine at position 512 (S512) is leucine or methionine; the amino acid that has been substituted for asparagine at position 514 (N514) is aspartic acid; the amino acid that has been substituted for asparagine at position 515 (N515) is phenylalanine, arginine, tryptophan, tyrosine, or lysine; the amino acid that has been substituted for glycine at position 569 (G569) is lysine, proline, or valine; and the amino acid that has been substituted for phenylalanine at position 596 (F596) is glycine.

[19] A modified β-galactosidase, wherein the modified β-galactosidase has undergone any of the following modifications in a β-galactosidase consisting of the amino acid sequence of any of SEQ ID NOs. 1 to 4 or an amino acid sequence having 90% or more identity to the amino acid sequence set forth in any of SEQ ID NOs. 1 to 4:
a double substitution in which the serine at position 188 or an amino acid corresponding thereto is substituted with isoleucine and the asparagine at position 550 or an amino acid corresponding thereto is substituted with phenylalanine (S188I N550F);
a double substitution in which the glutamine at position 407 or an amino acid corresponding thereto is substituted with arginine and the asparagine at position 550 or an amino acid corresponding thereto is substituted with phenylalanine (Q407R_N550F);
a double substitution in which the tyrosine at position 449 or an amino acid corresponding thereto is substituted with glutamine and the tyrosine at position 598 or an amino acid corresponding thereto is substituted with histidine (Y449Q_Y598H);
a double substitution in which the asparagine at position 550 or an amino acid corresponding thereto is substituted with phenylalanine and the proline at position 602 or an amino acid corresponding thereto is substituted with glutamine (N550F_P602Q);
a double substitution in which the glutamine at position 407 or an amino acid corresponding thereto is substituted with arginine and the tyrosine at position 449 or an amino acid corresponding thereto is substituted with glutamic acid (Q407R_Y449E);
a double substitution in which the asparagine at position 550 or an amino acid corresponding thereto is substituted with phenylalanine and the tyrosine at position 598 or an amino acid corresponding thereto is substituted with asparagine (N550F_Y598N);
a double substitution in which the proline at position 182 or an amino acid corresponding thereto is substituted with leucine and the tyrosine at position 449 or an amino acid corresponding thereto is substituted with glutamic acid (P182L_Y449E);
a double substitution in which the proline at position 182 or an amino acid corresponding thereto is substituted with leucine and the tyrosine at position 449 or an amino acid corresponding thereto is substituted with glutamine (P182L_Y449Q);
a double substitution in which the tyrosine at position 187 or an amino acid corresponding thereto is substituted with leucine and the glutamine at position 407 or an amino acid corresponding thereto is substituted with arginine (Y187L_Q407R);
a double substitution in which the tyrosine at position 187 or an amino acid corresponding thereto is substituted with leucine and the tyrosine at position 449 or an amino acid corresponding thereto is substituted with glutamic acid (Y187L_Y449E);
a double substitution in which the tyrosine at position 187 or an amino acid corresponding thereto is substituted with leucine and the tyrosine at position 449 or an amino acid corresponding thereto is substituted with glutamine (Y187L_Y449Q);
a double substitution in which the tyrosine at position 187 or an amino acid corresponding thereto is substituted with leucine and the serine at position 512 or an amino acid corresponding thereto is substituted with cysteine (Y187L_S512C);
a double substitution in which the tyrosine at position 187 or an amino acid corresponding thereto is substituted with leucine and the serine at position 512 or an amino acid corresponding thereto is substituted with asparagine (Y187L_S512N);
a double substitution in which the tyrosine at position 187 or an amino acid corresponding thereto is substituted with leucine and the serine at position 512 or an amino acid corresponding thereto is substituted with threonine (Y187L_S512T);
a double substitution in which the serine at position 188 or an amino acid corresponding thereto is substituted with isoleucine and the glutamine at position 407 or an amino acid corresponding thereto is substituted with arginine (S188I_Q407R);
a double substitution in which the serine at position 188 or an amino acid corresponding thereto is substituted with isoleucine and the tyrosine at position 449 or an amino acid corresponding thereto is substituted with glutamine (S188I_Y449Q);
a double substitution in which the serine at position 188 or an amino acid corresponding thereto is substituted with isoleucine and the threonine at position 483 or an amino acid corresponding thereto is substituted with glutamic acid (S188I_T483E);
a double substitution in which the glutamine at position 407 or an amino acid corresponding thereto is substituted with leucine and the serine at position 512 or an amino acid corresponding thereto is substituted with valine (Q407L_S512V);
a double substitution in which the glutamine at position 407 or an amino acid corresponding thereto is substituted with arginine and the threonine at position 483 or an amino acid corresponding thereto is substituted with glycine (Q407R_T483G);
a double substitution in which the glutamine at position 407 or an amino acid corresponding thereto is substituted with arginine and the tyrosine at position 449 or an amino acid corresponding thereto is substituted with glutamine (Q407R_Y449Q);
a double substitution in which the glutamine at position 407 or an amino acid corresponding thereto is substituted with arginine and the asparagine at position 550 or an amino acid corresponding thereto is substituted with serine (Q407R_N550S);
a double substitution in which the tyrosine at position 449 or an amino acid corresponding thereto is substituted with glutamine and the asparagine at position 550 or an amino acid corresponding thereto is substituted with serine (Y449Q_N550S);
a double substitution in which the lysine at position 487 or an amino acid corresponding thereto is substituted with tryptophan and the serine at position 512 or an amino acid corresponding thereto is substituted with threonine (K487W_S512T);
a double substitution in which the lysine at position 487 or an amino acid corresponding thereto is substituted with tryptophan and the aspartic acid at position 517 or an amino acid corresponding thereto is substituted with lysine (K487W_D517K);
a double substitution in which the lysine at position 487 or an amino acid corresponding thereto is substituted with tryptophan and the asparagine at position 515 or an amino acid corresponding thereto is substituted with glycine (K487W_N515G);
a double substitution in which the lysine at position 487 or an amino acid corresponding thereto is substituted with tryptophan and the aspartic acid at position 517 or an amino acid corresponding thereto is substituted with tryptophan (K487W_D517W);
a double substitution in which the lysine at position 487 or an amino acid corresponding thereto is substituted with proline and the asparagine at position 515 or an amino acid corresponding thereto is substituted with arginine (K487P N515R);
a double substitution in which the phenylalanine at position 91 or an amino acid corresponding thereto is substituted with serine and the glutamine at position 407 or an amino acid corresponding thereto is substituted with methionine (F91S_Q407M);
a double substitution in which the threonine at position 180 or an amino acid corresponding thereto is substituted with valine and the tyrosine at position 187 or an amino acid corresponding thereto is substituted with leucine (T180V_Y187L);
a double substitution in which the threonine at position 180 or an amino acid corresponding thereto is substituted with valine and the serine at position 188 or an amino acid corresponding thereto is substituted with isoleucine (T180V_S188I);
a double substitution in which the threonine at position 180 or an amino acid corresponding thereto is substituted with valine and the glutamine at position 407 or an amino acid corresponding thereto is substituted with arginine (T180V_Q407R);
a double substitution in which the threonine at position 180 or an amino acid corresponding thereto is substituted with valine and the tyrosine at position 449 or an amino acid corresponding thereto is substituted with glutamic acid (T180V_Y449E);
a double substitution in which the threonine at position 180 or an amino acid corresponding thereto is substituted with valine and the threonine at position 483 or an amino acid corresponding thereto is substituted with glycine (T180V_T483G);
a double substitution in which the threonine at position 180 or an amino acid corresponding thereto is substituted with valine and the serine at position 512 or an amino acid corresponding thereto is substituted with valine (T180V_S512V);
a double substitution in which the threonine at position 180 or an amino acid corresponding thereto is substituted with valine and the asparagine at position 550 or an amino acid corresponding thereto is substituted with phenylalanine (T180V_N550F);
a double substitution in which the threonine at position 180 or an amino acid corresponding thereto is substituted with valine and the tyrosine at position 615 or an amino acid corresponding thereto is substituted with proline (T180V_Y615P);
a double substitution in which the proline at position 182 or an amino acid corresponding thereto is substituted with leucine and the arginine at position 376 or an amino acid corresponding thereto is substituted with proline (P182L_R376P);
a double substitution in which the proline at position 182 or an amino acid corresponding thereto is substituted with tryptophan and the tyrosine at position 449 or an amino acid corresponding thereto is substituted with glutamine (P182W_Y449Q);
a double substitution in which the tyrosine at position 187 or an amino acid corresponding thereto is substituted with leucine and the lysine at position 487 or an amino acid corresponding thereto is substituted with alanine (Y187L_K487A);
a double substitution in which the tyrosine at position 187 or an amino acid corresponding thereto is substituted with leucine and the serine at position 512 or an amino acid corresponding thereto is substituted with alanine (Y187L_S512A);
a double substitution in which the tyrosine at position 187 or an amino acid corresponding thereto is substituted with leucine and the threonine at position 533 or an amino acid corresponding thereto is substituted with asparagine (Y187L_T533N);
a double substitution in which the arginine at position 376 or an amino acid corresponding thereto is substituted with proline and the glutamine at position 407 or an amino acid corresponding thereto is substituted with leucine (R376P_Q407L);
a double substitution in which the arginine at position 376 or an amino acid corresponding thereto is substituted with proline and the tyrosine at position 449 or an amino acid corresponding thereto is substituted with glutamine (R376P_Y449Q);
a double substitution in which the arginine at position 376 or an amino acid corresponding thereto is substituted with proline and the serine at position 512 or an amino acid corresponding thereto is substituted with valine (R376P_S512V);
a double substitution in which the arginine at position 376 or an amino acid corresponding thereto is substituted with proline and the asparagine at position 550 or an amino acid corresponding thereto is substituted with tryptophan (R376P_N550W);
a double substitution in which the arginine at position 376 or an amino acid corresponding thereto is substituted with proline and the asparagine at position 550 or an amino acid corresponding thereto is substituted with tyrosine (R376P_N550Y);
a double substitution in which the glutamine at position 407 or an amino acid corresponding thereto is substituted with leucine and the threonine at position 533 or an amino acid corresponding thereto is substituted with glutamic acid (Q407L_T533E);
a double substitution in which the glutamine at position 407 or an amino acid corresponding thereto is substituted with leucine and the tryptophan at position 570 or an amino acid corresponding thereto is substituted with tyrosine (Q407L_W570Y);
a double substitution in which the glutamine at position 407 or an amino acid corresponding thereto is substituted with leucine and the tyrosine at position 615 or an amino acid corresponding thereto is substituted with leucine (Q407L_Y615L);
a double substitution in which the glutamine at position 407 or an amino acid corresponding thereto is substituted with arginine and the tyrosine at position 615 or an amino acid corresponding thereto is substituted with leucine (Q407R_Y615L);
a double substitution in which the glutamine at position 407 or an amino acid corresponding thereto is substituted with arginine and the tyrosine at position 615 or an amino acid corresponding thereto is substituted with proline (Q407R_Y615P);
a double substitution in which the tyrosine at position 449 or an amino acid corresponding thereto is substituted with aspartic acid and the tyrosine at position 598 or an amino acid corresponding thereto is substituted with phenylalanine (Y449D_Y598F);
a double substitution in which the tyrosine at position 449 or an amino acid corresponding thereto is substituted with glutamine and the threonine at position 533 or an amino acid corresponding thereto is substituted with glutamic acid (Y449Q_T533E);
a double substitution in which the tyrosine at position 449 or an amino acid corresponding thereto is substituted with glutamine and the tyrosine at position 615 or an amino acid corresponding thereto is substituted with glycine (Y449Q_Y615G);
a double substitution in which the tyrosine at position 449 or an amino acid corresponding thereto is substituted with glutamine and the tyrosine at position 615 or an amino acid corresponding thereto is substituted with proline (Y449Q_Y615P);
a double substitution in which the threonine at position 483 or an amino acid corresponding thereto is substituted with glycine and the serine at position 512 or an amino acid corresponding thereto is substituted with valine (T483G_S512V);
a double substitution in which the threonine at position 483 or an amino acid corresponding thereto is substituted with glycine and the asparagine at position 550 or an amino acid corresponding thereto is substituted with serine (T483G_N550S);
a double substitution in which the threonine at position 483 or an amino acid corresponding thereto is substituted with glycine and the tyrosine at position 615 or an amino acid corresponding thereto is substituted with leucine (T483G_Y615L);
a double substitution in which the threonine at position 483 or an amino acid corresponding thereto is substituted with glutamine and the proline at position 602 or an amino acid corresponding thereto is substituted with glutamine (T483Q_P602Q);
a double substitution in which the arginine at position 484 or an amino acid corresponding thereto is substituted with histidine and the lysine at position 487 or an amino acid corresponding thereto is substituted with histidine (R484H_K487H);
a double substitution in which the arginine at position 484 or an amino acid corresponding thereto is substituted with tryptophan and the lysine at position 487 or an amino acid corresponding thereto is substituted with aspartic acid (R484W_K487D);
a double substitution in which the arginine at position 484 or an amino acid corresponding thereto is substituted with tryptophan and the threonine at position 575 or an amino acid corresponding thereto is substituted with tryptophan (R484W_T575W);
a double substitution in which the glycine at position 485 or an amino acid corresponding thereto is substituted with lysine and the valine at position 488 or an amino acid corresponding thereto is substituted with tryptophan (G485K_V488W);
a double substitution in which the glycine at position 485 or an amino acid corresponding thereto is substituted with lysine and the tyrosine at position 516 or an amino acid corresponding thereto is substituted with tryptophan (G485K_Y516W);
a double substitution in which the glycine at position 485 or an amino acid corresponding thereto is substituted with lysine and the aspartic acid at position 517 or an amino acid corresponding thereto is substituted with lysine (G485K_D517K);
a double substitution in which the glycine at position 485 or an amino acid corresponding thereto is substituted with lysine and the aspartic acid at position 517 or an amino acid corresponding thereto is substituted with tryptophan (G485K_D517W);
a double substitution in which the glycine at position 485 or an amino acid corresponding thereto is substituted with lysine and the glycine at position 569 or an amino acid corresponding thereto is substituted with tryptophan (G485K_G569W);
a double substitution in which the glycine at position 485 or an amino acid corresponding thereto is substituted with lysine and the glycine at position 571 or an amino acid corresponding thereto is substituted with lysine (G485K_G571K);
a double substitution in which the glycine at position 485 or an amino acid corresponding thereto is substituted with lysine and the arginine at position 572 or an amino acid corresponding thereto is substituted with tryptophan (G485K_R572W);
a double substitution in which the glycine at position 485 or an amino acid corresponding thereto is substituted with lysine and the threonine at position 573 or an amino acid corresponding thereto is substituted with tryptophan (G485K_T573W);
a double substitution in which the glycine at position 485 or an amino acid corresponding thereto is substituted with tryptophan and the tyrosine at position 516 or an amino acid corresponding thereto is substituted with tryptophan (G485W_Y516W);
a double substitution in which the glycine at position 485 or an amino acid corresponding thereto is substituted with tryptophan and the aspartic acid at position 517 or an amino acid corresponding thereto is substituted with tryptophan (G485W_D517W);
a double substitution in which the glycine at position 485 or an amino acid corresponding thereto is substituted with tryptophan and the threonine at position 573 or an amino acid corresponding thereto is substituted with lysine (G485W_T573K);
a double substitution in which the glycine at position 485 or an amino acid corresponding thereto is substituted with tryptophan and the threonine at position 573 or an amino acid corresponding thereto is substituted with tryptophan (G485W_T573W);
a double substitution in which the lysine at position 487 or an amino acid corresponding thereto is substituted with alanine and the serine at position 512 or an amino acid corresponding thereto is substituted with valine (K487A_S512V);
a double substitution in which the lysine at position 487 or an amino acid corresponding thereto is substituted with glycine and the asparagine at position 515 or an amino acid corresponding thereto is substituted with glutamine (K487G N515Q);
a double substitution in which the lysine at position 487 or an amino acid corresponding thereto is substituted with tryptophan and the threonine at position 573 or an amino acid corresponding thereto is substituted with lysine (K487W_T573K);
a double substitution in which the lysine at position 487 or an amino acid corresponding thereto is substituted with tryptophan and the tryptophan at position 593 or an amino acid corresponding thereto is substituted with phenylalanine (K487W_W593F);
a double substitution in which the valine at position 488 or an amino acid corresponding thereto is substituted with lysine and the asparagine at position 514 or an amino acid corresponding thereto is substituted with lysine (V488K_N514K);
a double substitution in which the valine at position 488 or an amino acid corresponding thereto is substituted with lysine and the aspartic acid at position 517 or an amino acid corresponding thereto is substituted with lysine (V488K_D517K);
a double substitution in which the valine at position 488 or an amino acid corresponding thereto is substituted with lysine and the glycine at position 569 or an amino acid corresponding thereto is substituted with lysine (V488K_G569K);
a double substitution in which the valine at position 488 or an amino acid corresponding thereto is substituted with lysine and the glycine at position 569 or an amino acid corresponding thereto is substituted with tryptophan (V488K_G569W);
a double substitution in which the valine at position 488 or an amino acid corresponding thereto is substituted with lysine and the arginine at position 572 or an amino acid corresponding thereto is substituted with lysine (V488K_R572K);
a double substitution in which the valine at position 488 or an amino acid corresponding thereto is substituted with lysine and the threonine at position 573 or an amino acid corresponding thereto is substituted with lysine (V488K_T573K);
a double substitution in which the valine at position 488 or an amino acid corresponding thereto is substituted with lysine and the threonine at position 573 or an amino acid corresponding thereto is substituted with tryptophan (V488K_T573W);
a double substitution in which the valine at position 488 or an amino acid corresponding thereto is substituted with lysine and the tyrosine at position 516 or an amino acid corresponding thereto is substituted with lysine (V488K_Y516K);
a double substitution in which the valine at position 488 or an amino acid corresponding thereto is substituted with lysine and the tyrosine at position 516 or an amino acid corresponding thereto is substituted with tryptophan (V488K_Y516W);
a double substitution in which the valine at position 488 or an amino acid corresponding thereto is substituted with tryptophan and the asparagine at position 514 or an amino acid corresponding thereto is substituted with tryptophan (V488W N514W);
a double substitution in which the valine at position 488 or an amino acid corresponding thereto is substituted with tryptophan and the threonine at position 573 or an amino acid corresponding thereto is substituted with tryptophan (V488W_T573W);
a double substitution in which the glutamic acid at position 500 or an amino acid corresponding thereto is substituted with alanine and the isoleucine at position 501 or an amino acid corresponding thereto is substituted with alanine (E500A_I501A);
a double substitution in which the serine at position 512 or an amino acid corresponding thereto is substituted with valine and the asparagine at position 550 or an amino acid corresponding thereto is substituted with serine (S512V_N550S);
a double substitution in which the threonine at position 533 or an amino acid corresponding thereto is substituted with glutamic acid and the asparagine at position 550 or an amino acid corresponding thereto is substituted with serine (T533E_N550S);
a double substitution in which the threonine at position 533 or an amino acid corresponding thereto is substituted with glutamic acid and the tyrosine at position 615 or an amino acid corresponding thereto is substituted with glycine (T533E_Y615G);
a double substitution in which the tryptophan at position 570 or an amino acid corresponding thereto is substituted with tyrosine and the tyrosine at position 615 or an amino acid corresponding thereto is substituted with leucine (W570Y_Y615L);
a double substitution in which the phenylalanine at position 91 or an amino acid corresponding thereto is substituted with valine and the glutamic acid at position 513 or an amino acid corresponding thereto is substituted with glycine (F91V_E513G);
a double substitution in which the threonine at position 180 or an amino acid corresponding thereto is substituted with valine and the threonine at position 483 or an amino acid corresponding thereto is substituted with lysine (T180V_T483K);
a double substitution in which the threonine at position 180 or an amino acid corresponding thereto is substituted with valine and the tyrosine at position 598 or an amino acid corresponding thereto is substituted with asparagine (T180V_Y598N);
a double substitution in which the threonine at position 180 or an amino acid corresponding thereto is substituted with valine and the tyrosine at position 615 or an amino acid corresponding thereto is substituted with glycine (T180V_Y615G);
a double substitution in which the proline at position 182 or an amino acid corresponding thereto is substituted with leucine and the tyrosine at position 615 or an amino acid corresponding thereto is substituted with leucine (P182L_Y615L);
a double substitution in which the arginine at position 376 or an amino acid corresponding thereto is substituted with proline and the tyrosine at position 598 or an amino acid corresponding thereto is substituted with asparagine (R376P_Y598N);
a double substitution in which the glutamine at position 407 or an amino acid corresponding thereto is substituted with leucine and the threonine at position 483 or an amino acid corresponding thereto is substituted with lysine (Q407L_T483K);
a double substitution in which the glutamine at position 407 or an amino acid corresponding thereto is substituted with leucine and the lysine at position 487 or an amino acid corresponding thereto is substituted with proline (Q407L_K487P);
a double substitution in which the glutamine at position 407 or an amino acid corresponding thereto is substituted with leucine and the tyrosine at position 615 or an amino acid corresponding thereto is substituted with glycine (Q407L_Y615G);
a double substitution in which the tyrosine at position 449 or an amino acid corresponding thereto is substituted with glutamic acid and the lysine at position 487 or an amino acid corresponding thereto is substituted with alanine (Y449E_K487A);
a double substitution in which the tyrosine at position 449 or an amino acid corresponding thereto is substituted with glutamic acid and the lysine at position 487 or an amino acid corresponding thereto is substituted with proline (Y449E_K487P);
a double substitution in which the threonine at position 483 or an amino acid corresponding thereto is substituted with glycine and the lysine at position 487 or an amino acid corresponding thereto is substituted with alanine (T483G_K487A);
a double substitution in which the threonine at position 483 or an amino acid corresponding thereto is substituted with glycine and the threonine at position 533 or an amino acid corresponding thereto is substituted with glutamic acid (T483G_T533E);
a double substitution in which the threonine at position 483 or an amino acid corresponding thereto is substituted with glycine and the tyrosine at position 615 or an amino acid corresponding thereto is substituted with glycine (T483G_Y615G);
a double substitution in which the threonine at position 483 or an amino acid corresponding thereto is substituted with methionine and the lysine at position 487 or an amino acid corresponding thereto is substituted with alanine (T483M_K487A);
a double substitution in which the threonine at position 483 or an amino acid corresponding thereto is substituted with methionine and the lysine at position 487 or an amino acid corresponding thereto is substituted with glycine (T483M_K487G);
a double substitution in which the threonine at position 483 or an amino acid corresponding thereto is substituted with methionine and the lysine at position 487 or an amino acid corresponding thereto is substituted with proline (T483M_K487P);
a double substitution in which the arginine at position 484 or an amino acid corresponding thereto is substituted with histidine and the lysine at position 487 or an amino acid corresponding thereto is substituted with aspartic acid (R484H_K487D);
a double substitution in which the arginine at position 484 or an amino acid corresponding thereto is substituted with lysine and the glutamic acid at position 513 or an amino acid corresponding thereto is substituted with tryptophan (R484K_E513W);
a double substitution in which the arginine at position 484 or an amino acid corresponding thereto is substituted with lysine and the asparagine at position 514 or an amino acid corresponding thereto is substituted with lysine (R484K_N514K);
a double substitution in which the arginine at position 484 or an amino acid corresponding thereto is substituted with lysine and the asparagine at position 514 or an amino acid corresponding thereto is substituted with tryptophan (R484K_N514W);
a double substitution in which the arginine at position 484 or an amino acid corresponding thereto is substituted with lysine and the asparagine at position 515 or an amino acid corresponding thereto is substituted with lysine (R484K_N515K);
a double substitution in which the arginine at position 484 or an amino acid corresponding thereto is substituted with lysine and the asparagine at position 515 or an amino acid corresponding thereto is substituted with tryptophan (R484K_N515W);
a double substitution in which the arginine at position 484 or an amino acid corresponding thereto is substituted with lysine and the aspartic acid at position 517 or an amino acid corresponding thereto is substituted with tryptophan (R484K_D517W);
a double substitution in which the arginine at position 484 or an amino acid corresponding thereto is substituted with lysine and the glycine at position 569 or an amino acid corresponding thereto is substituted with lysine (R484K_G569K);
a double substitution in which the arginine at position 484 or an amino acid corresponding thereto is substituted with lysine and the glycine at position 571 or an amino acid corresponding thereto is substituted with lysine (R484K_G571K);
a double substitution in which the arginine at position 484 or an amino acid corresponding thereto is substituted with lysine and the arginine at position 572 or an amino acid corresponding thereto is substituted with lysine (R484K_R572K);
a double substitution in which the arginine at position 484 or an amino acid corresponding thereto is substituted with lysine and the arginine at position 572 or an amino acid corresponding thereto is substituted with tryptophan (R484K_R572W);
a double substitution in which the arginine at position 484 or an amino acid corresponding thereto is substituted with lysine and the threonine at position 573 or an amino acid corresponding thereto is substituted with lysine (R484K_T573K);
a double substitution in which the arginine at position 484 or an amino acid corresponding thereto is substituted with lysine and the threonine at position 573 or an amino acid corresponding thereto is substituted with tryptophan (R484K_T573W);
a double substitution in which the arginine at position 484 or an amino acid corresponding thereto is substituted with lysine and the tyrosine at position 516 or an amino acid corresponding thereto is substituted with lysine (R484K_Y516K);
a double substitution in which the arginine at position 484 or an amino acid corresponding thereto is substituted with tryptophan and the asparagine at position 515 or an amino acid corresponding thereto is substituted with lysine (R484W_N515K);
a double substitution in which the glycine at position 485 or an amino acid corresponding thereto is substituted with aspartic acid and the lysine at position 487 or an amino acid corresponding thereto is substituted with histidine (G485D_K487H);
a double substitution in which the glycine at position 485 or an amino acid corresponding thereto is substituted with lysine and the lysine at position 487 or an amino acid corresponding thereto is substituted with histidine (G485K_K487H);
a double substitution in which the glycine at position 485 or an amino acid corresponding thereto is substituted with lysine and the glycine at position 569 or an amino acid corresponding thereto is substituted with lysine (G485K_G569K);
a double substitution in which the glycine at position 485 or an amino acid corresponding thereto is substituted with lysine and the asparagine at position 514 or an amino acid corresponding thereto is substituted with lysine (G485K_N514K);
a double substitution in which the glycine at position 485 or an amino acid corresponding thereto is substituted with lysine and the asparagine at position 515 or an amino acid corresponding thereto is substituted with lysine (G485K_N515K);
a double substitution in which the glycine at position 485 or an amino acid corresponding thereto is substituted with lysine and the asparagine at position 515 or an amino acid corresponding thereto is substituted with tryptophan (G485K_N515W);
a double substitution in which the glycine at position 485 or an amino acid corresponding thereto is substituted with arginine and the lysine at position 487 or an amino acid corresponding thereto is substituted with aspartic acid (G485R_K487D);
a double substitution in which the glycine at position 485 or an amino acid corresponding thereto is substituted with tryptophan and the glutamic acid at position 513 or an amino acid corresponding thereto is substituted with tryptophan (G485W_E513W);
a double substitution in which the glycine at position 485 or an amino acid corresponding thereto is substituted with tryptophan and the asparagine at position 515 or an amino acid corresponding thereto is substituted with lysine (G485W_N515K);
a double substitution in which the glycine at position 485 or an amino acid corresponding thereto is substituted with tryptophan and the asparagine at position 515 or an amino acid corresponding thereto is substituted with tryptophan (G485W N515W);
a double substitution in which the lysine at position 487 or an amino acid corresponding thereto is substituted with alanine and the serine at position 531 or an amino acid corresponding thereto is substituted with alanine (K487A_S531A);
a double substitution in which the lysine at position 487 or an amino acid corresponding thereto is substituted with alanine and the tryptophan at position 593 or an amino acid corresponding thereto is substituted with phenylalanine (K487A_W593F);
a double substitution in which the lysine at position 487 or an amino acid corresponding thereto is substituted with alanine and the tyrosine at position 615 or an amino acid corresponding thereto is substituted with leucine (K487A_Y615L);
a double substitution in which the lysine at position 487 or an amino acid corresponding thereto is substituted with tryptophan and the asparagine at position 514 or an amino acid corresponding thereto is substituted with lysine (K487W_N514K);
a double substitution in which the lysine at position 487 or an amino acid corresponding thereto is substituted with tryptophan and the asparagine at position 514 or an amino acid corresponding thereto is substituted with tryptophan (K487W N514W);
a double substitution in which the lysine at position 487 or an amino acid corresponding thereto is substituted with tryptophan and the tyrosine at position 516 or an amino acid corresponding thereto is substituted with tryptophan (K487W_Y516W);
a double substitution in which the lysine at position 487 or an amino acid corresponding thereto is substituted with tryptophan and the glycine at position 569 or an amino acid corresponding thereto is substituted with lysine (K487W_G569K);
a double substitution in which the lysine at position 487 or an amino acid corresponding thereto is substituted with tyrosine and the asparagine at position 515 or an amino acid corresponding thereto is substituted with phenylalanine (K487Y_N515F);
a double substitution in which the lysine at position 487 or an amino acid corresponding thereto is substituted with tyrosine and the asparagine at position 515 or an amino acid corresponding thereto is substituted with tryptophan (K487Y N515W);
a double substitution in which the valine at position 488 or an amino acid corresponding thereto is substituted with lysine and the asparagine at position 515 or an amino acid corresponding thereto is substituted with lysine (V488K_N515K);
a double substitution in which the valine at position 488 or an amino acid corresponding thereto is substituted with lysine and the asparagine at position 515 or an amino acid corresponding thereto is substituted with tryptophan (V488K_N515W);
a double substitution in which the valine at position 488 or an amino acid corresponding thereto is substituted with tryptophan and the asparagine at position 515 or an amino acid corresponding thereto is substituted with lysine (V488W_N515K);
a double substitution in which the valine at position 488 or an amino acid corresponding thereto is substituted with tryptophan and the asparagine at position 515 or an amino acid corresponding thereto is substituted with tryptophan (V488W N515W);
a double substitution in which the valine at position 488 or an amino acid corresponding thereto is substituted with tyrosine and the asparagine at position 515 or an amino acid corresponding thereto is substituted with tryptophan (V488Y N515W);
a double substitution in which the asparagine at position 550 or an amino acid corresponding thereto is substituted with serine and the tyrosine at position 598 or an amino acid corresponding thereto is substituted with asparagine (N550S_Y598N);
a double substitution in which the asparagine at position 550 or an amino acid corresponding thereto is substituted with serine and the tyrosine at position 615 or an amino acid corresponding thereto is substituted with glycine (N550S_Y615G);
a double substitution in which the asparagine at position 550 or an amino acid corresponding thereto is substituted with serine and the tyrosine at position 615 or an amino acid corresponding thereto is substituted with leucine (N550S_Y615L);
a double substitution in which the asparagine at position 550 or an amino acid corresponding thereto is substituted with serine and the tyrosine at position 615 or an amino acid corresponding thereto is substituted with proline (N550S_Y615P);
a triple substitution in which the arginine at position 484 or an amino acid corresponding thereto is substituted with alanine, the glycine at position 485 or an amino acid corresponding thereto is substituted with alanine, and the lysine at position 487 or an amino acid corresponding thereto is substituted with aspartic acid (R484A_G485A_K487D);
a triple substitution in which the arginine at position 484 or an amino acid corresponding thereto is substituted with aspartic acid, the glycine at position 485 or an amino acid corresponding thereto is substituted with aspartic acid, and the lysine at position 487 or an amino acid corresponding thereto is substituted with aspartic acid (R484D_G485D_K487D);
a triple substitution in which the glycine at position 485 or an amino acid corresponding thereto is substituted with arginine, the aspartic acid at position 486 or an amino acid corresponding thereto is substituted with arginine, and the lysine at position 487 or an amino acid corresponding thereto is substituted with arginine (G485R_D486R_K487R);
a quadruple substitution in which the arginine at position 484 or an amino acid corresponding thereto is substituted with alanine, the glycine at position 485 or an amino acid corresponding thereto is substituted with alanine, the aspartic acid at position 486 or an amino acid corresponding thereto is substituted with alanine, and the lysine at position 487 or an amino acid corresponding thereto is substituted with alanine (R484A_G485A_D486A_K487A);
a quadruple substitution in which the arginine at position 484 or an amino acid corresponding thereto is substituted with alanine, the glycine at position 485 or an amino acid corresponding thereto is substituted with alanine, the aspartic acid at position 486 or an amino acid corresponding thereto is substituted with proline, and the lysine at position 487 or an amino acid corresponding thereto is substituted with proline (R484A_G485A_D486P_K487P);
a triple substitution in which the arginine at position 484 or an amino acid corresponding thereto is substituted with alanine, the glycine at position 485 or an amino acid corresponding thereto is substituted with alanine, and the lysine at position 487 or an amino acid corresponding thereto is substituted with aspartic acid (R484A_G485A_K487D);
a triple substitution in which the arginine at position 484 or an amino acid corresponding thereto is substituted with cysteine, the aspartic acid at position 486 or an amino acid corresponding thereto is substituted with histidine, and the lysine at position 487 or an amino acid corresponding thereto is substituted with arginine (R484C_D486H_K487R);
a triple substitution in which the glycine at position 485 or an amino acid corresponding thereto is substituted with arginine, the aspartic acid at position 486 or an amino acid corresponding thereto is substituted with tryptophan, and the lysine at position 487 or an amino acid corresponding thereto is substituted with tryptophan (G485R_D486W_K487W);
a triple substitution in which the glycine at position 485 or an amino acid corresponding thereto is substituted with arginine, the aspartic acid at position 486 or an amino acid corresponding thereto is substituted with alanine, and the lysine at position 487 or an amino acid corresponding thereto is substituted with alanine (G485R_D486A_K487A);
a triple substitution in which the glycine at position 485 or an amino acid corresponding thereto is substituted with arginine, the aspartic acid at position 486 or an amino acid corresponding thereto is substituted with proline, and the lysine at position 487 or an amino acid corresponding thereto is substituted with proline (G485R_D486P_K487P);
a quadruple substitution in which the arginine at position 484 or an amino acid corresponding thereto is substituted with alanine, the glycine at position 485 or an amino acid corresponding thereto is substituted with alanine, the aspartic acid at position 486 or an amino acid corresponding thereto is substituted with alanine, and the lysine at position 487 or an amino acid corresponding thereto is substituted with alanine (R484A_G485A_D486A_K487A);
a quadruple substitution in which the arginine at position 484 or an amino acid corresponding thereto is substituted with alanine, the glycine at position 485 or an amino acid corresponding thereto is substituted with alanine, the aspartic acid at position 486 or an amino acid corresponding thereto is substituted with arginine, and the lysine at position 487 or an amino acid corresponding thereto is substituted with arginine (R484A_G485A_D486R_K487R);
a quadruple substitution in which the arginine at position 484 or an amino acid corresponding thereto is substituted with aspartic acid, the glycine at position 485 or an amino acid corresponding thereto is substituted with aspartic acid, the aspartic acid at position 486 or an amino acid corresponding thereto is substituted with arginine, and the lysine at position 487 or an amino acid corresponding thereto is substituted with arginine (R484D_G485D_D486R_K487R);
a quadruple substitution in which the arginine at position 484 or an amino acid corresponding thereto is substituted with aspartic acid, the glycine at position 485 or an amino acid corresponding thereto is substituted with aspartic acid, the aspartic acid at position 486 or an amino acid corresponding thereto is substituted with proline, and the lysine at position 487 or an amino acid corresponding thereto is substituted with proline (R484D_G485D_D486P_K487P);
a quadruple substitution in which the arginine at position 484 or an amino acid corresponding thereto is substituted with aspartic acid, the glycine at position 485 or an amino acid corresponding thereto is substituted with aspartic acid, the aspartic acid at position 486 or an amino acid corresponding thereto is substituted with alanine, and the lysine at position 487 or an amino acid corresponding thereto is substituted with alanine (R484D_G485D_D486A_K487A);
a quadruple substitution in which the arginine at position 484 or an amino acid corresponding thereto is substituted with aspartic acid, the glycine at position 485 or an amino acid corresponding thereto is substituted with aspartic acid, the aspartic acid at position 486 or an amino acid corresponding thereto is substituted with tryptophan, and the lysine at position 487 or an amino acid corresponding thereto is substituted with tryptophan (R484D_G485D_D486W_K487W); and
a quadruple substitution in which the arginine at position 484 or an amino acid corresponding thereto is substituted with proline, the glycine at position 485 or an amino acid corresponding thereto is substituted with proline, the aspartic acid at position 486 or an amino acid corresponding thereto is substituted with arginine, and the lysine at position 487 or an amino acid corresponding thereto is substituted with arginine (R484P_G485P_D486R_K487R).

[20] A modified β-galactosidase, wherein the modified β-galactosidase has undergone any of the following modifications in a β-galactosidase consisting of the amino acid sequence of any of SEQ ID NOs. 1 to 4 or an amino acid sequence having 90% or more identity to the amino acid sequence set forth in any of SEQ ID NOs. 1 to 4:
(a) a deletion of the region of arginine at position 484 (R484) to lysine at position 487 (K487) or a region corresponding thereto;
(b) an insertion of an amino acid(s) at the position between asparagine at position 514 (N514) and asparagine at position 515 (N515) or at a position corresponding thereto;
(c) an insertion of an amino acid(s) at the position between proline at position 604 (P604) and tyrosine at position 605 (Y605) or at a position corresponding thereto;
(d) a deletion of the glycine at position 485 (G485) or an amino acid corresponding thereto, and in addition, a substitution of aspartic acid at position 486 (D486) or an amino acid corresponding thereto;
(e) a deletion of the region of glycine at position 485 (G485) to aspartic acid at position 486 (D486) or a region corresponding thereto, and in addition, a substitution of lysine at position 487 (K487) or an amino acid corresponding thereto;
(f) a deletion of the region of glycine at position 485 (G485) to aspartic acid at position 486 (D486) or a region corresponding thereto, and in addition, a substitution of arginine at position 484 (R484) or an amino acid corresponding thereto, and a substitution of lysine at position 487 (K487) or an amino acid corresponding thereto;
(g) an insertion of an amino acid(s) at the position between arginine at position 484 (R484) and glycine at position 485 (G485) or at a position corresponding thereto; and
(h) an insertion of an amino acid(s) at the position between glycine at position 485 (G485) and aspartic acid at position 486 (D486) or at a position corresponding thereto.

[21] The modified β-galactosidase according to [20], wherein for (d) to (f), the amino acid that has been substituted for arginine at position 484 (R484) is tryptophan or proline, the amino acid that has been substituted for aspartic acid at position 486 (D486) is histidine, and the amino acid that has been substituted for lysine at position 487 (K487) is arginine, tryptophan, or proline; and
for (b), (c), (g), and (h), the number of amino acids to be inserted is from 1 to 10.

[22] A modified β-galactosidase, wherein the modified β-galactosidase has undergone any of the following modifications in a β-galactosidase consisting of the amino acid sequence of SEQ ID NO. 1:
(i) a substitution of the lysine at position 487 (K487) with glycine or proline;
(ii) a substitution of the asparagine at position 514 (N514) with tryptophan; and
(iii) a substitution of the asparagine at position 515 (N515) with lysine or tryptophan.

[23] A gene coding the modified β-galactosidase according to any one of [1] to [22].

[24] A recombinant DNA comprising the gene according to [23].

[25] A microorganism carrying the recombinant DNA according to [24].

[26] An enzyme preparation comprising the modified β-galactosidase according to any one of [1] to [22].

[27] A method for the production of an oligosaccharide, characterized in that the modified β-galactosidase according to any one of [1] to [22] is allowed to act on a disaccharide, oligosaccharide, or polysaccharide having at least one of β-1,3-, β-1,4-, and β-1,6-bonds.

### BRIEF DESCRIPTION OF DRAWINGS

FIG. 1 shows a comparison of a β-galactosidase from Bacillus circulans and 3GM8 (a beta-glycosidase from Bacteroides vulgatus (Identities=282/824 (34%), Positives=425/824 (52%)).

FIG. 2 shows a system for recombinant expression of variants (modified β-galactosidases).

FIG. 3 shows variants with decreased or increased hydrolytic activities for allolactose. (A) Measurement results for variants meeting the condition that allolactose decomposing activity÷1%-lactose decomposing activity is 50% or less. (B) Measurement results for variants meeting a condition that allolactose decomposing activity÷1%-lactose decomposing activity is 150% or greater. WT: wild-type enzyme.

FIG. 4 shows variants by which the content of tetrasaccharides in the composition of the galacto-oligosaccharides produced was altered. WT: wild-type enzyme.

FIG. 5 shows variants by which the content of allolactose in the composition of the galacto-oligosaccharides produced was altered. WT: wild-type enzyme.

FIG. 6 shows variants by which the content of tetrasaccharides in the composition of the galacto-oligosaccharides produced was altered. WT: wild-type enzyme.

FIG. 7 shows variants with single amino acid substitution and meeting condition 3 (see below). WT: wild-type enzyme.

FIG. 8 shows variants with single amino acid substitution and meeting condition 4 (see below). WT: wild-type enzyme.

FIG. 9 shows variants with single amino acid substitution which produced oligosaccharides with an increased content of tetrasaccharides. WT-A: wild-type enzyme of BgaD type A.

FIG. 10 shows variants in which a substitution meeting condition 3 (see below) and a substitution meeting condition 5 (see below) were combined (case 1), a substitution meeting condition 3 (see below) and a substitution meeting condition 6 (see below) were combined (case 3), a substitution meeting condition 4 (see below) and a substitution meeting condition 5 (see below) were combined (case 4), substitutions meeting condition 3 (see below) were combined (case 5-1), and substitutions meeting condition 4 (see below) were combined (case 5-2). WT: wild-type enzyme.

FIG. 11 shows variants with two amino acid substitutions and meeting condition 3 (see below; left), and variants with three or four amino acid substitutions and meeting condition 4 (see below; right). WT: wild-type enzyme.

FIG. 12 shows variants with three or four amino acid substitutions and meeting condition 3 (see below; upper) and condition 4 (see below; bottom). WT: wild-type enzyme.

FIG. 13 shows insertion or deletion variants and meeting condition 3 (see below; upper) and condition 4 (see below; bottom). WT: wild-type enzyme.

### DESCRIPTION OF EMBODIMENTS

For convenience of description, some of the terms used in relation to the present invention are defined as follows.

### (Terminology)

The term “modified β-galactosidase” refers to an enzyme obtained by modification or mutation of a particular β-galactosidase (which is referred to as a “reference β-galactosidase” for convenience of description). The reference β-galactosidase is a β-galactosidase produced by Bacillus circulans. From earlier studies, it is known that the β-galactosidase from Bacillus circulans is made up of four enzymes (isozymes) with different molecular weights. The present invention uses any of these four enzymes (isozymes) as a reference β-galactosidase. Typically, an enzyme having the amino acid sequence of any of SEQ ID NOs. 1 to 4 is used as a reference β-galactosidase. However, it is also possible to as a reference β-galactosidase, use an enzyme that has an amino acid sequence having 90% or more identity to the amino acid sequence of any of SEQ ID NOs. 1 to 4, wherein the enzyme exhibits β-galactosidase activity. As a reference β-galactosidase, it is possible to preferably use an enzyme that has an amino acid sequence having 95% or more identity to the amino acid sequence of any of SEQ ID NOs. 1 to 4, wherein the enzyme exhibits β-galactosidase activity, more preferably an enzyme that has an amino acid sequence having 98% or more identity to the amino acid sequence of any of SEQ ID NOs. 1 to 4, wherein the enzyme exhibits β-galactosidase activity, and most preferably an enzyme that has an amino acid sequence having 99% or more identity to the amino acid sequence of any of SEQ ID NOs. 1 to 4, wherein the enzyme exhibits β-galactosidase activity. SEQ ID NO. 1 represents the amino acid sequence of an isozyme of β-galactosidase with a molecular weight of 195 kD (also referred to hereinafter as BgaD-A); SEQ ID NO. 2 represents the amino acid sequence of an isozyme of β-galactosidase with a molecular weight of 160 kD (also referred to hereinafter as BgaD-B); SEQ ID NO. 3 represents the amino acid sequence of an isozyme of β-galactosidase with a molecular weight of 135 kD (also referred to hereinafter as BgaD-C); and SEQ ID NO. 4 represents the amino acid sequence of an isozyme of β-galactosidase with a molecular weight of 86 kD (also referred to hereinafter as BgaD-D). The base sequences coding BgaD-A, BgaD-B, BgaD-C, and BgaD-D are represented in SEQ ID NOs. 5, 6, 7, and 8, respectively.

In the present invention, an “amino acid substitution” is carried out as modification or mutation. Therefore, some amino acid residues are found to be different when a modified β-galactosidase and a reference β-galactosidase therefor are compared. In the specification, a modified β-galactosidase is also referred to as a modified enzyme, an variant β-galactosidase, a variant, or others.

In the specification, amino acids are designated according to the common practice, as their single letters as described below:
methionine: M; serine: S; alanine: A; threonine: T; valine: V; tyrosine: Y; leucine: L; asparagine: N; isoleucine: I; glutamine: Q; proline: P; aspartic acid: D; phenylalanine: F; glutamic acid: E; tryptophan: W; lysine: K; cysteine: C; arginine: R; glycine: G; and histidine: H.

In addition, an amino acid residue at a mutation site (an amino acid residue to be substituted with another amino acid) is expressed in a combination of the above-described single letter representing the kind of the amino acid residue and the figure representing the position of the amino acid residue. For example, if proline at position 182 is a mutation site, then the amino acid is designated as “P182.”

(Modified β-galactosidases)

A first aspect of the present invention is directed to a modified β-galactosidase (modified enzyme). A modified enzyme of the present invention typically has an amino acid sequence in which one or more amino acids selected from the group consisting of the following (1) to (21), in a β-galactosidase consisting of the amino acid sequence of any of SEQ ID NOs. 1 to 4 or an amino acid sequence having 90% or more identity to the amino acid sequence set forth in any of SEQ ID NOs. 1 to 4:
(1) proline at position 182 (P182),
(2) tyrosine at position 187 (Y187),
(3) serine at position 188 (S188),
(4) tryptophan at position 405 (W405),
(5) alanine at position 406 (A406),
(6) glutamine at position 407 (Q407),
(7) tyrosine at position 449 (Y449),
(8) threonine at position 483 (T483),
(9) serine at position 512 (S512),
(10) serine at position 531 (S531),
(11) threonine at position 533 (T533),
(12) serine at position 534 (S534),
(13) asparagine at position 550 (N550),
(14) glutamine at position 551 (Q551),
(15) tryptophan at position 593 (W593),
(16) tyrosine at position 598 (Y598),
(17) proline at position 602 (P602),
(18) proline at position 604 (P604),
(19) tyrosine at position 609 (Y609),
(20) lysine at position 612 (K612), and
(21) tyrosine at position 615 (Y615).

As shown in the examples described below, the above-described amino acid residues to be substituted have been identified by prediction of the steric structure of the wild-type enzymes and validation experiments on variant enzymes. In an embodiment of the present invention, the structure of a reference β-galactosidase enzyme is modified by substitution of one or more of these amino acids to alter a property of the reference β-galactosidase. Here, the “property” is an activity to synthesize oligosaccharides and/or a hydrolytic activity for allolactose.

In a reference β-galactosidase that has an amino acid sequence having 90% or more identity, preferably 95% or more identity, more preferably 98% or more identity, most preferably 99% or more identity, to the amino acid sequence of any of SEQ ID NOs. 1 to 4, insertion or deletion of an amino acid(s) may result in differences in the positions of amino acids between the sequences of the reference β-galactosidase and an enzyme consisting of the amino acid sequence of any of SEQ ID NOs. 1 to 4. For this reason, in one embodiment of the present invention, an amino acid(s) corresponding to one or more amino acids selected from the group consisting of the above-described amino acids (1) to (21) in a reference β-galactosidase is/are to be substituted. Here, the term “corresponding” means that between two proteins (enzymes) to be compared, the corresponding amino acids in the two proteins make a comparable contribution in exerting their function. For example, it is possible that when an amino acid sequence to be compared is aligned relative to a reference amino acid sequence (for example, the amino acid sequence of SEQ ID NO. 1) with taking into account a partial homology in their primary structures (amino acid sequences), whereby an optimal comparison between them can be made (in this case, the alignment can be optimized by insertion of a gap or gaps if needed), an amino acid at the position corresponding to that of a given amino acid in the reference amino acid sequence is identified as an “amino acid corresponding” thereto. Instead of or in addition to making a comparison between the primary structures of two proteins (enzymes), an “amino acid corresponding” in the two proteins (enzymes) can also be identified by making a comparison between their steric structures (three-dimensional structures). The use of steric structure information allows one to obtain comparison results with high reliability. In this case, a procedure can be employed in which the alignment is carried out with comparing the atomic coordinates in the steric structures of a plurality of enzymes.

A kind of an amino acid after substitution is not particularly limited. Thus, it may be “conservative amino acid substitution” or “non-conservative amino acid substitution”. The “conservative amino acid substitution” herein refers to substituting a certain amino acid residue with an amino acid residue having a side chain with the same characteristics. Amino acid residues are classified into some families according to their side chains, such as basic side chains (e.g., lysine, arginine, and histidine), acidic side chains (e.g., asparaginic acid, and glutamic acid), uncharged polar side chains (e.g., glycine, asparagine, glutamine, serine, threonine, tyrosine, and cysteine), nonpolar side chains (e.g., alanine, valine, leucine, isoleucine, proline, phenylalanine, methionine, and tryptophan), β-branched side chains (e.g., threonine, valine, and isoleucine), and aromatic side chains (e.g., tyrosine, phenylalanine, tryptophan, and histidine). The conservative amino acid substitution is typically substitution between amino acid residues in the same family.

In a first embodiment of the present invention, the one or more amino acids to be substituted are selected from the group consisting of tyrosine at position 449 (Y449), serine at position 531 (S531), tryptophan at position 593 (W593), proline at position 602 (P602), proline at position 604 (P604), and lysine at position 612 (K612). These amino acids have been identified as meeting the condition that “a variant enzyme has an allolactose decomposing activity of 50% or less relative to that of the parent wild-type enzyme” (condition 1), in experiments described in the Examples which follows, and are related to the substrate specificity of the hydrolytic activity for allolactose. Accordingly, it can be said that modified enzymes in this embodiment are highly useful in that they have a reduced allolactose-hydrolytic activity. The amino acids that have been substituted for the above-mentioned amino acids are preferably as indicated below. An amino acid to be substituted in this embodiment is referred to as a “group 1 amino acid.”

The amino acid that has been substituted for tyrosine at position 449 (Y449) is aspartic acid, glutamic acid, asparagine, or tryptophan.

The amino acid that has been substituted for serine at position 531 (S531) is alanine, aspartic acid, glutamic acid, phenylalanine, glycine, isoleucine, methionine, asparagine, or glutamine.

The amino acid that has been substituted for tryptophan at position 593 (W593) is phenylalanine.

The amino acid that has been substituted for proline at position 602 (P602) is glutamic acid or glycine.

The amino acid that has been substituted for proline at position 604 (P604) is alanine, glycine, histidine, isoleucine, methionine, serine, threonine, valine, tryptophan, or tyrosine.

The amino acid that has been substituted for lysine at position 612 (K612) is methionine.

In a second embodiment of the present invention, the one or more amino acids to be substituted are selected from the group consisting of tyrosine at position 187 (Y187), serine at position 188 (S188), threonine at position 533 (T533), proline at position 602 (P602), and lysine at position 612 (K612). These amino acids have been identified as meeting the condition that “a variant enzyme has an allolactose decomposing activity of 150% or greater relative to that of the parent wild-type enzyme” (condition 2), in experiments described in the Examples which follows, and are related to the substrate specificity of the hydrolytic activity for allolactose. Accordingly, it can be said that modified enzymes in this embodiment are highly useful in that they have an enhanced allolactose-hydrolytic activity. The amino acids that have been substituted for the above-mentioned amino acids are preferably as indicated below. An amino acid to be substituted in this embodiment is referred to as a “group 2 amino acid.”

The amino acid that has been substituted for tyrosine at position 187 (Y187) is aspartic acid, glutamic acid, or glycine.

The amino acid that has been substituted for serine at position 188 (S188) is proline or threonine.

The amino acid that has been substituted for threonine at position 533 (T533) is methionine or glutamine.

The amino acid that has been substituted for proline at position 602 (P602) is cysteine, isoleucine, asparagine, serine, threonine, or valine.

The amino acid that has been substituted for lysine at position 612 (K612) is phenylalanine.

In a third embodiment of the present invention, the one or more amino acids to be substituted are selected from the group consisting of proline at position 182 (P182), tyrosine at position 187 (Y187), serine at position 188 (S188), tryptophan at position 405 (W405), glutamine at position 407 (Q407), tyrosine at position 449 (Y449), threonine at position 483 (T483), serine at position 512 (S512), serine at position 531 (S531), asparagine at position 550 (N550), proline at position 602 (P602), and proline at position 604 (P604). These amino acids have been identified as meeting the condition that “a variant enzyme produces oligosaccharides in which the content of trisaccharides and higher saccharides is approximately equal and the content of tetrasaccharides is smaller, relative to the case of the parent wild-type enzyme” (condition 3), in experiments described in the Examples which follows, and are related to the amount of production of tetrasaccharides. Accordingly, it can be said that modified enzymes in this embodiment are highly useful in that their use results in a decrease in the content of tetrasaccharides. The amino acids that have been substituted for the above-mentioned amino acids are preferably as indicated below. I An amino acid to be substituted in this embodiment is referred to as a “group 3 amino acid.”

The amino acid that has been substituted for proline at position 182 (P182) is leucine.

The amino acid that has been substituted for tyrosine at position 187 (Y187) is aspartic acid or leucine.

The amino acid that has been substituted for serine at position 188 (S188) is isoleucine.

The amino acid that has been substituted for tryptophan at position 405 (W405) is alanine, aspartic acid, glutamic acid, glycine, histidine, leucine, asparagine, serine, or threonine.

The amino acid that has been substituted for glutamine at position 407 (Q407) is glycine or arginine.

The amino acid that has been substituted for tyrosine at position 449 (Y449) is glutamic acid, isoleucine, glutamine, arginine, or serine.

The amino acid that has been substituted for threonine at position 483 (T483) is glutamine.

The amino acid that has been substituted for serine at position 512 (S512) is phenylalanine, threonine, or valine. The amino acid that has been substituted for serine at position 531 (S531) is proline.

The amino acid that has been substituted for asparagine at position 550 (N550) is leucine, arginine, or serine.

The amino acid that has been substituted for proline at position 602 (P602) is leucine, asparagine, or glutamine.

The amino acid that has been substituted for proline at position 604 (P604) is isoleucine or methionine.

From the results of further studies, as amino acids to be substituted wichi meet the condition that “a variant enzyme produces oligosaccharides in which the content of trisaccharides and higher saccharides is approximately equal and the content of tetrasaccharides is smaller, relative to the case of the parent wild-type enzyme” (condition 3) (i.e., group 3 amino acids), there have been identified tryptophan at position 85 (W85), phenylalanine at position 91 (F91), tyrosine at position 103 (Y103), tyrosine at position 134 (Y134), tyrosine at position 153 (Y153), proline at position 182 (P182), tryptophan at position 186 (W186), tyrosine at position 192 (Y192), tyrosine at position 295 (Y295), histidine at position 345 (H345), glycine at position 349 (G349), phenylalanine at position 402 (F402), glutamine at position 407 (Q407), glycine at position 479 (G479), glutamic acid at position 480 (E480), lysine at position 482 (K482), threonine at position 483 (T483), arginine at position 484 (R484), glycine at position 485 (G485), aspartic acid at position 486 (D486), lysine at position 487 (K487), valine at position 488 (V488), tyrosine at position 496 (Y496), serine at position 512 (S512), glutamic acid at position 513 (E513), asparagine at position 514 (N514), asparagine at position 515 (N515), tyrosine at position 516 (Y516), aspartic acid at position 517 (D517), tyrosine at position 519 (Y519), threonine at position 533 (T533), tyrosine at position 547 (Y547), tyrosine at position 549 (Y549), tyrosine at position 555 (Y555), arginine at position 572 (R572), threonine at position 573 (T573), tyrosine at position 598 (Y598), tyrosine at position 605 (Y605), tyrosine at position 606 (Y606), tyrosine at position 609 (Y609), phenylalanine at position 616 (F616), phenylalanine at position 624 (F624), tryptophan at position 648 (W648), and tryptophan at position 650 (W650). Therefore, one embodiment of the present invention provides a modified enzyme consisting of an amino acid sequence in which one or more amino acids selected from the group consisting of the above-mentioned amino acids, or an amino acid(s) corresponding thereto, in a β-galactosidase consisting of the amino acid sequence of any of SEQ ID NOs. 1 to 4 or an amino acid sequence having 90% or more identity to the amino acid sequence set forth in any of SEQ ID NOs. 1 to 4. The amino acids that have been substituted for the above-mentioned amino acids are preferably as indicated below.

The amino acid that has been substituted for tryptophan at position 85 (W85) is glycine.

The amino acid that has been substituted for phenylalanine at position 91 (F91) is alanine or isoleucine.

The amino acid that has been substituted for tyrosine at position 103 (Y103) is glycine.

The amino acid that has been substituted for tyrosine at position 134 (Y134) is glycine.

The amino acid that has been substituted for tyrosine at position 153 (Y153) is glycine.

The amino acid that has been substituted for proline at position 182 (P182) is tryptophan or tyrosine.

The amino acid that has been substituted for tryptophan at position 186 (W186) is methionine.

The amino acid that has been substituted for tyrosine at position 192 (Y192) is glycine.

The amino acid that has been substituted for tyrosine at position 295 (Y295) is glycine.

The amino acid that has been substituted for histidine at position 345 (H345) is glycine.

The amino acid that has been substituted for glycine at position 349 (G349) is tryptophan.

The amino acid that has been substituted for phenylalanine at position 402 (F402) is glycine.

The amino acid that has been substituted for glutamine at position 407 (Q407) is isoleucine, leucine, methionine, valine, or tyrosine.

The amino acid that has been substituted for glycine at position 479 (G479) is leucine, proline, or glutamine.

The amino acid that has been substituted for glutamic acid at position 480 (E480) is asparagine.

The amino acid that has been substituted for lysine at position 482 (K482) is methionine or valine.

The amino acid that has been substituted for threonine at position 483 (T483) is glycine or serine.

The amino acid that has been substituted for arginine at position 484 (R484) is phenylalanine, isoleucine, methionine, asparagine, or glutamine.

The amino acid that has been substituted for glycine at position 485 (G485) is aspartic acid, asparagine, glutamine, or threonine.

The amino acid that has been substituted for aspartic acid at position 486 (D486) is alanine, glutamic acid, phenylalanine, isoleucine, lysine, leucine, methionine, proline, glutamine, arginine, threonine, tryptophan, or tyrosine.

The amino acid that has been substituted for lysine at position 487 (K487) is cysteine, glutamic acid, phenylalanine, histidine, isoleucine, leucine, asparagine, glutamine, arginine, threonine, valine, tryptophan, or tyrosine.

The amino acid that has been substituted for valine at position 488 (V488) is glycine, isoleucine, leucine, arginine, serine, threonine, tryptophan, or tyrosine.

The amino acid that has been substituted for tyrosine at position 496 (Y496) is glycine.

The amino acid that has been substituted for serine at position 512 (S512) is cysteine, aspartic acid, glutamic acid, asparagine, proline, glutamine, or tryptophan.

The amino acid that has been substituted for glutamic acid at position 513 (E513) is alanine, cysteine, aspartic acid, phenylalanine, glycine, histidine, lysine, leucine, methionine, asparagine, proline, glutamine, arginine, serine, threonine, or valine.

The amino acid that has been substituted for asparagine at position 514 (N514) is glutamic acid, leucine, or lysine.

The amino acid that has been substituted for asparagine at position 515 (N515) is alanine, aspartic acid, glutamic acid, glycine, isoleucine, leucine, proline, or valine.

The amino acid that has been substituted for tyrosine at position 516 (Y516) is tryptophan.

The amino acid that has been substituted for aspartic acid at position 517 (D517) is lysine or tryptophan.

The amino acid that has been substituted for tyrosine at position 519 (Y519) is glycine.

The amino acid that has been substituted for threonine at position 533 (T533) is glutamic acid.

The amino acid that has been substituted for tyrosine at position 547 (Y547) is glycine.

The amino acid that has been substituted for tyrosine at position 549 (Y549) is glycine.

The amino acid that has been substituted for tyrosine at position 555 (Y555) is glycine.

The amino acid that has been substituted for arginine at position 572 (R572) is cysteine, leucine, methionine, threonine, tryptophan, or tyrosine.

The amino acid that has been substituted for threonine at position 573 (T573) is glutamic acid.

The amino acid that has been substituted for tyrosine at position 598 (Y598) is histidine or asparagine.

The amino acid that has been substituted for tyrosine at position 605 (Y605) is cysteine, aspartic acid, histidine, isoleucine, lysine, proline, arginine, or threonine.

The amino acid that has been substituted for tyrosine at position 606 (Y606) is alanine, cysteine, aspartic acid, glutamic acid, phenylalanine, glycine, histidine, isoleucine, lysine, leucine, methionine, or serine.

The amino acid that has been substituted for tyrosine at position 609 (Y609) is glutamic acid, asparagine, or serine.

The amino acid that has been substituted for phenylalanine at position 616 (F616) is glycine.

The amino acid that has been substituted for phenylalanine at position 624 (F624) is glycine.

The amino acid that has been substituted for tryptophan at position 648 (W648) is glycine.

The amino acid that has been substituted for tryptophan at position 650 (W650) is glycine.

In a fourth embodiment of the present invention, the one or more amino acids to be substituted are selected from the group consisting of threonine at position 483 (T483), serine at position 512 (S512), glutamine at position 551 (Q551), tyrosine at position 598 (Y598), and tyrosine at position 615 (Y615). These amino acids have been identified as meeting the condition that “a variant enzyme produces oligosaccharides in which the content of trisaccharides and higher saccharides is approximately equal and the content of tetrasaccharides is higher, relative to the case of the parent wild-type enzyme” (condition 4), in experiments described in the Examples which follow, and are related to the amount of production of tetrasaccharides. Accordingly, it can be said that modified enzymes in this embodiment are highly useful in that their use results in an increase in the content of tetrasaccharides. The amino acids that have been substituted for the above-mentioned amino acids are preferably as indicated below. An amino acid to be substituted in the fourth embodiment is referred to as a “group 4 amino acid.”

The amino acid that has been substituted for threonine at position 483 (T483) is lysine, methionine, or arginine.

The amino acid that has been substituted for serine at position 512 (S512) is glycine or lysine.

The amino acid that has been substituted for glutamine at position 551 (Q551) is asparagine.

The amino acid that has been substituted for tyrosine at position 598 (Y598) is asparagine.

The amino acid that has been substituted for tyrosine at position 615 (Y615) is threonine.

From the results of further studies, as the amino acids that are to be substituted and that meet the condition that “a variant enzyme produces oligosaccharides in which the content of trisaccharides and higher saccharides is approximately equal and the content of tetrasaccharides is higher, relative to the case of the parent wild-type enzyme” (condition 4) (i.e., group 4 amino acids), there have been identified tryptophan at position 76 (W76), glutamic acid at position 480 (E480), threonine at position 483 (T483), glycine at position 485 (G485), arginine at position 484 (R484), lysine at position 487 (K487), valine at position 488 (V488), serine at position 512 (S512), asparagine at position 514 (N514), asparagine at position 515 (N515), glycine at position 569 (G569), and phenylalanine at position 596 (F596). Therefore, one embodiment of the present invention provides a modified enzyme consisting of an amino acid sequence in which one or more amino acids selected from the group consisting of the above-mentioned amino acids, or an amino acid(s) corresponding thereto, has/have been substituted by other amino acid in a β-galactosidase consisting of the amino acid sequence of any of SEQ ID NOs. 1 to 4 or an amino acid sequence having 90% or more identity to the amino acid sequence set forth in any of SEQ ID NOs. 1 to 4. The amino acids that have been substituted for the above-mentioned amino acids are preferably as indicated below.

The amino acid that has been substituted for tryptophan at position 76 (W76) is glycine.

The amino acid that has been substituted for glutamic acid at position 480 (E480) is glutamine.

The amino acid that has been substituted for threonine at position 483 (T483) is tryptophan.

The amino acid that has been substituted for glycine at position 485 (G485) is proline or threonine.

The amino acid that has been substituted for arginine at position 484 (R484) is alanine, cysteine, aspartic acid, glycine, proline, serine, tyrosine, or lysine.

The amino acid that has been substituted for lysine at position 487 (K487) is glycine or proline.

The amino acid that has been substituted for valine at position 488 (V488) is proline.

The amino acid that has been substituted for serine at position 512 (S512) is leucine or methionine.

The amino acid that has been substituted for asparagine at position 514 (N514) is aspartic acid.

The amino acid that has been substituted for asparagine at position 515 (N515) is phenylalanine, arginine, tryptophan, tyrosine, or lysine.

The amino acid that has been substituted for glycine at position 569 (G569) is lysine, proline, or valine.

The amino acid that has been substituted for phenylalanine at position 596 (F596) is glycine.

In a fifth embodiment of the present invention, the one or more amino acids to be substituted are selected from the group consisting of tryptophan at position 405 (W405), threonine at position 483 (T483), serine at position 531 (S531), asparagine at position 550 (N550), and tyrosine at position 598 (Y598). These amino acids have been identified as meeting the condition that “a variant enzyme produces oligosaccharides in which the content of trisaccharides and higher saccharides is approximately equal and the content of allolactose is smaller, relative to the case of the parent wild-type enzyme” (condition 5), in experiments described in the Examples which follow, and are related to the content of allolactose.

Accordingly, it can be said that modified enzymes in this embodiment are highly useful in that their use results in a decrease in the percentage of allolactose. The amino acids that have been substituted for the above-mentioned amino acids are preferably as indicated below. An amino acid to be substituted in the fifth embodiment is referred to as a “group 5 amino acid.”

The amino acid that has been substituted for tryptophan at position 405 (W405) is alanine, aspartic acid, serine, or threonine.

The amino acid that has been substituted for threonine at position 483 (T483) is methionine.

The amino acid that has been substituted for serine at position 531 (S531) is aspartic acid, phenylalanine, glycine, leucine, lysine, or methionine.

The amino acid that has been substituted for asparagine at position 550 (N550) is phenylalanine.

The amino acid that has been substituted for tyrosine at position 598 (Y598) is phenylalanine or histidine.

In a sixth embodiment of the present invention, the one or more amino acids to be substituted are selected from the group consisting of proline at position 182 (P182), tyrosine at position 187 (Y187), serine at position 188 (S188), tryptophan at position 405 (W405), alanine at position 406 (A406), glutamine at position 407 (Q407), tyrosine at position 449 (Y449), threonine at position 483 (T483), serine at position 512 (S512), serine at position 534 (S534), asparagine at position 550 (N550), glutamine at position 551 (Q551), proline at position 602 (P602), tyrosine at position 609 (Y609), lysine at position 612 (K612), and tyrosine at position 615 (Y615). These amino acids have been identified as meeting the condition that “a variant enzyme produces oligosaccharides in which the content of trisaccharides and higher saccharides is approximately equal and the content of allolactose is higher, relative to the case of the parent wild-type enzyme” (condition), in experiments described in the Examples which follow, and are related to the content of allolactose. Accordingly, it can be said that modified enzymes in this embodiment are highly useful in that their use results in an increase in the percentage of allolactose. The amino acids that have been substituted for the above-mentioned amino acids are preferably as indicated below. An amino acid to be substituted in the sixth embodiment is referred to as a “group 6 amino acid.”

The amino acid that has been substituted for proline at position 182 (P182) is leucine or serine.

The amino acid that has been substituted for tyrosine at position 187 (Y187) is aspartic acid, glutamic acid, or asparagine.

The amino acid that has been substituted for serine at position 188 (S188) is glutamic acid, glycine, isoleucine, or asparagine.

The amino acid that has been substituted for tryptophan at position 405 (W405) is histidine.

The amino acid that has been substituted for alanine at position 406 (A406) is proline.

The amino acid that has been substituted for glutamine at position 407 (Q407) is alanine, phenylalanine, glycine, lysine, or arginine.

The amino acid that has been substituted for tyrosine at position 449 (Y449) is glutamic acid or arginine.

The amino acid that has been substituted for threonine at position 483 (T483) is serine or glutamine.

The amino acid that has been substituted for serine at position 512 (S512) is glycine, threonine, or valine.

The amino acid that has been substituted for serine at position 534 (S534) is lysine or glutamine.

The amino acid that has been substituted for asparagine at position 550 (N550) is alanine, glycine, lysine, leucine, methionine, arginine, or serine.

The amino acid that has been substituted for glutamine at position 551 (Q551) is lysine or histidine.

The amino acid that has been substituted for proline at position 602 (P602) is cysteine, aspartic acid, or asparagine.

The amino acid that has been substituted for tyrosine at position 609 (Y609) is aspartic acid, glutamine, or threonine.

The amino acid that has been substituted for lysine at position 612 (K612) is phenylalanine or isoleucine.

The amino acid that has been substituted for tyrosine at position 615 (Y615) is glutamic acid, isoleucine, or threonine.

In the present invention, two or more amino acids may be substituted. As mentioned above, the amino acids to be substituted are classified into groups 1 to 6. Two or more amino acids to be substituted are selected, for example, from among the amino acids within one group.

In this embodiment, two or more amino acids belonging to one group are substituted, whereby it is expected that modified enzymes are generated in which a property characteristic of the given group (for example, a reduced allolactose-decomposing activity in the case of the group 1 amino acids) is enhanced. On the other hand, two or more amino acids to be substituted may be selected from among the amino acids within two or more different groups. It is expected that such selections would lead to the generation of modified enzymes in which properties of the respective groups to which the selected amino acids belong are combined. Preferable specific examples in this embodiment are given below. In this context, although combinations of amino acids to be substituted are not limited in particular, it is preferable that amino acids from groups exhibiting opposite properties, that is, from groups 1 and 2, from groups 3 and 4, or from groups 5 and 6, are not combined.

### (Case 1)

Amino acids from group 3 and from group 5 are selected as targets for substitution. Modified enzymes obtained are expected to exhibit a characteristic of producing oligo-saccharides in which both the percentages of tetrasaccharides and of allolactose are reduced.

### (Case 2)

Amino acids from group 4 and from group 6 are selected as targets for substitution. Modified enzymes obtained are expected to exhibit a characteristic of producing oligo-saccharides in which both the percentages of tetrasaccharides and of allolactose are increased.

### (Case 3)

Amino acids from group 3 and from group 6 are selected as targets for substitution. Modified enzymes obtained are expected to exhibit a characteristic of producing oligo-saccharides in which the percentage of tetrasaccharides is reduced, whereas the percentage of allolactose is increased.

### (Case 4)

Amino acids from group 4 and from group 5 are selected as targets for substitution. Modified enzymes obtained are expected to exhibit a characteristic of producing oligo-saccharides in which the percentage of tetrasaccharides is increased, whereas the percentage of allolactose is reduced.

### (Case 5)

A plurality of amino acids from one group is substituted. Substitutions of amino acids for plurality of amino acids from one group are expected to bring about a greater alteration. For example, if a plurality of amino acids from among the group 3 amino acids is selected as targets for substitution, then modified enzymes obtained are expected to exhibit a characteristic of producing oligo-saccharides in which the percentage of tetrasaccharides is reduced to a greater degree.

Specific examples falling within the above-described case 1 can include a modified enzyme in which the serine at position 188 is substituted with isoleucine and the asparagine at position 550 is substituted with phenylalanine (S188I_N550F); a modified enzyme in which the glutamine at position 407 is substituted with arginine and the asparagine at position 550 is substituted with phenylalanine (Q407R_N550F); a modified enzyme in which the tyrosine at position 449 is substituted with glutamine and the tyrosine at position 598 is substituted with histidine (Y449Q_Y598H); and a modified enzyme in which the asparagine at position 550 is substituted with phenylalanine and the proline at position 602 is substituted with glutamine (N550F_P602Q).

Specific examples falling within the above-described case 3 can include a modified enzyme in which the glutamine at position 407 is substituted with arginine and the tyrosine at position 449 is substituted with glutamic acid (Q407R_Y449E).

Specific examples falling within the above-described case 4 can include a modified enzyme in which the asparagine at position 550 is substituted with phenylalanine and the tyrosine at position 598 is substituted with asparagine (N550F_Y598N).

Specific examples falling within the above-described case 5 (wherein a plurality of amino acids from among the group 3 amino acids are selected as targets for substitution) can include a modified enzyme in which the proline at position 182 is substituted with leucine and the tyrosine at position 449 is substituted with glutamic acid (P182L_Y449E); a modified enzyme in which the proline at position 182 is substituted with leucine and the tyrosine at position 449 is substituted with glutamine (P182L_Y449Q); a modified enzyme in which the tyrosine at position 187 is substituted with leucine and the glutamine at position 407 is substituted with arginine (Y187L_Q407R); a modified enzyme in which the tyrosine at position 187 is substituted with leucine and the tyrosine at position 449 is substituted with glutamic acid (Y187L_Y449E); a modified enzyme in which the tyrosine at position 187 is substituted with leucine and the tyrosine at position 449 is substituted with glutamine (Y187L_Y449Q); a modified enzyme in which the tyrosine at position 187 is substituted with leucine and the serine at position 512 is substituted with cysteine (Y187L_S512C); a modified enzyme in which the tyrosine at position 187 is substituted with leucine and the serine at position 512 is substituted with asparagine (Y187L_S512N); a modified enzyme in which the tyrosine at position 187 is substituted with leucine and the serine at position 512 is substituted with threonine (Y187L_S512T); a modified enzyme in which the serine at position 188 is substituted with isoleucine and the glutamine at position 407 is substituted with arginine (S188I_Q407R); a modified enzyme in which the serine at position 188 is substituted with isoleucine and the tyrosine at position 449 is substituted with glutamine (S188I_Y449Q); a modified enzyme in which the serine at position 188 is substituted with isoleucine and the threonine at position 483 is substituted with glutamic acid (S188I_T483E); a modified enzyme in which the glutamine at position 407 is substituted with leucine and the serine at position 512 is substituted with valine (Q407L_S512V); a modified enzyme in which the glutamine at position 407 is substituted with arginine and the threonine at position 483 is substituted with glycine (Q407R_T483G); a modified enzyme in which the glutamine at position 407 is substituted with arginine and the tyrosine at position 449 is substituted with glutamine (Q407R_Y449Q); a modified enzyme in which the glutamine at position 407 is substituted with arginine and the asparagine at position 550 is substituted with serine (Q407R_N550S); a modified enzyme in which the tyrosine at position 449 is substituted with glutamine and the asparagine at position 550 is substituted with serine (Y449Q_N550S); a modified enzyme in which the lysine at position 487 is substituted with tryptophan and the serine at position 512 is substituted with threonine (K487W_S512T); a modified enzyme in which the lysine at position 487 is substituted with tryptophan and the aspartic acid at position 517 is substituted with lysine (K487W_D517K); a modified enzyme in which the lysine at position 487 is substituted with tryptophan and the asparagine at position 515 is substituted with glycine (K487W_N515G); and a modified enzyme in which the lysine at position 487 is substituted with tryptophan and the aspartic acid at position 517 is substituted with tryptophan (K487W_D517W).

Similarly, specific examples falling within the above-described case 5 (wherein a plurality of amino acids from among the group 4 amino acids are selected as targets for substitution) can include a modified enzyme in which the lysine at position 487 is substituted with proline and the asparagine at position 515 is substituted with arginine (K487P_N515R).

Further specific examples of modified enzymes in which two or more amino acids are substituted can include a modified enzyme in which the phenylalanine at position 91 is substituted with serine and the glutamine at position 407 is substituted with methionine (F91S_Q407M), a modified enzyme in which the threonine at position 180 is substituted with valine and the tyrosine at position 187 is substituted with leucine (T180V_Y187L), a modified enzyme in which the threonine at position 180 is substituted with valine and the serine at position 188 is substituted with isoleucine (T180V_S188I), a modified enzyme in which the threonine at position 180 is substituted with valine and the glutamine at position 407 is substituted with arginine (T180V_Q407R), a modified enzyme in which the threonine at position 180 is substituted with valine and the tyrosine at position 449 is substituted with glutamic acid (T180V_Y449E), a modified enzyme in which the threonine at position 180 is substituted with valine and the threonine at position 483 is substituted with glycine (T180V_T483G), a modified enzyme in which the threonine at position 180 is substituted with valine and the serine at position 512 is substituted with valine (T180V_S512V), a modified enzyme in which the threonine at position 180 is substituted with valine and the asparagine at position 550 is substituted with phenylalanine (T180V N550F), a modified enzyme in which the threonine at position 180 is substituted with valine and the tyrosine at position 615 is substituted with proline (T180V_Y615P), a modified enzyme in which the proline at position 182 is substituted with leucine and the arginine at position 376 is substituted with proline (P182L_R376P), a modified enzyme in which the proline at position 182 is substituted with tryptophan and the tyrosine at position 449 is substituted with glutamine (P182W_Y449Q), a modified enzyme in which the tyrosine at position 187 is substituted with leucine and the lysine at position 487 is substituted with alanine (Y187L_K487A), a modified enzyme in which the tyrosine at position 187 is substituted with leucine and the serine at position 512 is substituted with alanine (Y187L_S512A), a modified enzyme in which the tyrosine at position 187 is substituted with leucine and the threonine at position 533 is substituted with asparagine (Y187L_T533N), a modified enzyme in which the arginine at position 376 is substituted with proline and the glutamine at position 407 is substituted with leucine (R376P_Q407L), a modified enzyme in which the arginine at position 376 is substituted with proline and the tyrosine at position 449 is substituted with glutamine (R376P_Y449Q), a modified enzyme in which the arginine at position 376 is substituted with proline and the serine at position 512 is substituted with valine (R376P_S512V), a modified enzyme in which the arginine at position 376 is substituted with proline and the asparagine at position 550 is substituted with tryptophan (R376P_N550W), a modified enzyme in which the arginine at position 376 is substituted with proline and the asparagine at position 550 is substituted with tyrosine (R376P_N550Y), a modified enzyme in which the glutamine at position 407 is substituted with leucine and the threonine at position 533 is substituted with glutamic acid (Q407L_T533E), a modified enzyme in which the glutamine at position 407 is substituted with leucine and the tryptophan at position 570 is substituted with tyrosine (Q407L_W570Y), a modified enzyme in which the glutamine at position 407 is substituted with leucine and the tyrosine at position 615 is substituted with leucine (Q407L_Y615L), a modified enzyme in which the glutamine at position 407 is substituted with arginine and the tyrosine at position 615 is substituted with leucine (Q407R_Y615L), a modified enzyme in which the glutamine at position 407 is substituted with arginine and the tyrosine at position 615 is substituted with proline (Q407R_Y615P), a modified enzyme in which the tyrosine at position 449 is substituted with aspartic acid and the tyrosine at position 598 is substituted with phenylalanine (Y449D_Y598F), a modified enzyme in which the tyrosine at position 449 is substituted with glutamine and the threonine at position 533 is substituted with glutamic acid (Y449Q_T533E), a modified enzyme in which the tyrosine at position 449 is substituted with glutamine and the tyrosine at position 615 is substituted with glycine (Y449Q_Y615G), a modified enzyme in which the tyrosine at position 449 is substituted with glutamine and the tyrosine at position 615 is substituted with proline (Y449Q_Y615P), a modified enzyme in which the threonine at position 483 is substituted with glycine and the serine at position 512 is substituted with valine (T483G_S512V), a modified enzyme in which the threonine at position 483 is substituted with glycine and the asparagine at position 550 is substituted with serine (T483G_N550S), a modified enzyme in which the threonine at position 483 is substituted with glycine and the tyrosine at position 615 is substituted with leucine (T483G Y615L), a modified enzyme in which the threonine at position 483 is substituted with glutamine and the proline at position 602 is substituted with glutamine (T483Q_P602Q), a modified enzyme in which the arginine at position 484 is substituted with histidine and the lysine at position 487 is substituted with histidine (R484H_K487H), a modified enzyme in which the arginine at position 484 is substituted with tryptophan and the lysine at position 487 is substituted with aspartic acid (R484W_K487D), a modified enzyme in which the arginine at position 484 is substituted with tryptophan and the threonine at position 575 is substituted with tryptophan (R484W_T575W), a modified enzyme in which the glycine at position 485 is substituted with lysine and the valine at position 488 is substituted with tryptophan (G485K_V488W), a modified enzyme in which the glycine at position 485 is substituted with lysine and the tyrosine at position 516 is substituted with tryptophan (G485K_Y516W), a modified enzyme in which the glycine at position 485 is substituted with lysine and the aspartic acid at position 517 is substituted with lysine (G485K_D517K), a modified enzyme in which the glycine at position 485 is substituted with lysine and the aspartic acid at position 517 is substituted with tryptophan (G485K_D517W), a modified enzyme in which the glycine at position 485 is substituted with lysine and the glycine at position 569 is substituted with tryptophan (G485K_G569W), a modified enzyme in which the glycine at position 485 is substituted with lysine and the glycine at position 571 is substituted with lysine (G485K_G571K), a modified enzyme in which the glycine at position 485 is substituted with lysine and the arginine at position 572 is substituted with tryptophan (G485K_R572W), a modified enzyme in which the glycine at position 485 is substituted with lysine and the threonine at position 573 is substituted with tryptophan (G485K_T573W), a modified enzyme in which the glycine at position 485 is substituted with tryptophan and the tyrosine at position 516 is substituted with tryptophan (G485W_Y516W), a modified enzyme in which the glycine at position 485 is substituted with tryptophan and the aspartic acid at position 517 is substituted with tryptophan (G485W_D517W), a modified enzyme in which the glycine at position 485 is substituted with tryptophan and the threonine at position 573 is substituted with lysine (G485W_T573K), a modified enzyme in which the glycine at position 485 is substituted with tryptophan and the threonine at position 573 is substituted with tryptophan (G485W_T573W), a modified enzyme in which the lysine at position 487 is substituted with alanine and the serine at position 512 is substituted with valine (K487A_S512V), a modified enzyme in which the lysine at position 487 is substituted with glycine and the asparagine at position 515 is substituted with glutamine (K487G_N515Q), a modified enzyme in which the lysine at position 487 is substituted with tryptophan and the threonine at position 573 is substituted with lysine (K487W_T573K), a modified enzyme in which the lysine at position 487 is substituted with tryptophan and the tryptophan at position 593 is substituted with phenylalanine (K487W_W593F), a modified enzyme in which the valine at position 488 is substituted with lysine and the asparagine at position 514 is substituted with lysine (V488K N514K), a modified enzyme in which the valine at position 488 is substituted with lysine and the aspartic acid at position 517 is substituted with lysine (V488K_D517K), a modified enzyme in which the valine at position 488 is substituted with lysine and the glycine at position 569 is substituted with lysine (V488K_G569K), a modified enzyme in which the valine at position 488 is substituted with lysine and the glycine at position 569 is substituted with tryptophan (V488K_G569W), a modified enzyme in which the valine at position 488 is substituted with lysine and the arginine at position 572 is substituted with lysine (V488K_R572K), a modified enzyme in which the valine at position 488 is substituted with lysine and the threonine at position 573 is substituted with lysine (V488K_T573K), a modified enzyme in which the valine at position 488 is substituted with lysine and the threonine at position 573 is substituted with tryptophan (V488K_T573W), a modified enzyme in which the valine at position 488 is substituted with lysine and the tyrosine at position 516 is substituted with lysine (V488K_Y516K), a modified enzyme in which the valine at position 488 is substituted with lysine and the tyrosine at position 516 is substituted with tryptophan (V488K_Y516W), a modified enzyme in which the valine at position 488 is substituted with tryptophan and the asparagine at position 514 is substituted with tryptophan (V488W_N514W), a modified enzyme in which the valine at position 488 is substituted with tryptophan and the threonine at position 573 is substituted with tryptophan (V488W_T573W), a modified enzyme in which the glutamic acid at position 500 is substituted with alanine and the isoleucine at position 501 is substituted with alanine (E500A_I501A), a modified enzyme in which the serine at position 512 is substituted with valine and the asparagine at position 550 is substituted with serine (S512V_N550S), a modified enzyme in which the threonine at position 533 is substituted with glutamic acid and the asparagine at position 550 is substituted with serine (T533E N550S), a modified enzyme in which the threonine at position 533 is substituted with glutamic acid and the tyrosine at position 615 is substituted with glycine (T533E_Y615G), a modified enzyme in which the tryptophan at position 570 is substituted with tyrosine and the tyrosine at position 615 is substituted with leucine (W570Y_Y615L) (these modified enzymes meet the above-described condition 3 that “a variant enzyme produces oligosaccharides in which the content of trisaccharides and higher saccharides is approximately equal and the content of tetrasaccharides is smaller, relative to the case of the parent wild-type enzyme”); a modified enzyme in which the phenylalanine at position 91 is substituted with valine and the glutamic acid at position 513 is substituted with glycine (F91V_E513G), a modified enzyme in which the threonine at position 180 is substituted with valine and the threonine at position 483 is substituted with lysine (T180V_T483K), a modified enzyme in which the threonine at position 180 is substituted with valine and the tyrosine at position 598 is substituted with asparagine (T180V_Y598N), a modified enzyme in which the threonine at position 180 is substituted with valine and the tyrosine at position 615 is substituted with glycine (T180V_Y615G), a modified enzyme in which the proline at position 182 is substituted with leucine and the tyrosine at position 615 is substituted with leucine (P182L_Y615L), a modified enzyme in which the arginine at position 376 is substituted with proline and the tyrosine at position 598 is substituted with asparagine (R376P_Y598N), a modified enzyme in which the glutamine at position 407 is substituted with leucine and the threonine at position 483 is substituted with lysine (Q407L_T483K), a modified enzyme in which the glutamine at position 407 is substituted with leucine and the lysine at position 487 is substituted with proline (Q407L_K487P), a modified enzyme in which the glutamine at position 407 is substituted with leucine and the tyrosine at position 615 is substituted with glycine (Q407L_Y615G), a modified enzyme in which the tyrosine at position 449 is substituted with glutamic acid and the lysine at position 487 is substituted with alanine (Y449E_K487A), a modified enzyme in which the tyrosine at position 449 is substituted with glutamic acid and the lysine at position 487 is substituted with proline (Y449E_K487P), a modified enzyme in which the threonine at position 483 is substituted with glycine and the lysine at position 487 is substituted with alanine (T483G K487A), a modified enzyme in which the threonine at position 483 is substituted with glycine and the threonine at position 533 is substituted with glutamic acid (T483G T533E), a modified enzyme in which the threonine at position 483 is substituted with glycine and the tyrosine at position 615 is substituted with glycine (T483G Y615G), a modified enzyme in which the threonine at position 483 is substituted with methionine and the lysine at position 487 is substituted with alanine (T483M_K487A), a modified enzyme in which the threonine at position 483 is substituted with methionine and the lysine at position 487 is substituted with glycine (T483M_K487G), a modified enzyme in which the threonine at position 483 is substituted with methionine and the lysine at position 487 is substituted with proline (T483M_K487P), a modified enzyme in which the arginine at position 484 is substituted with histidine and the lysine at position 487 is substituted with aspartic acid (R484H_K487D), a modified enzyme in which the arginine at position 484 is substituted with lysine and the glutamic acid at position 513 is substituted with tryptophan (R484K_E513W), a modified enzyme in which the arginine at position 484 is substituted with lysine and the asparagine at position 514 is substituted with lysine (R484K N514K), a modified enzyme in which the arginine at position 484 is substituted with lysine and the asparagine at position 514 is substituted with tryptophan (R484K_N514W), a modified enzyme in which the arginine at position 484 is substituted with lysine and the asparagine at position 515 is substituted with lysine (R484K_N515K), a modified enzyme in which the arginine at position 484 is substituted with lysine and the asparagine at position 515 is substituted with tryptophan (R484K_N515W), a modified enzyme in which the arginine at position 484 is substituted with lysine and the aspartic acid at position 517 is substituted with tryptophan (R484K_D517W), a modified enzyme in which the arginine at position 484 is substituted with lysine and the glycine at position 569 is substituted with lysine (R484K_G569K), a modified enzyme in which the arginine at position 484 is substituted with lysine and the glycine at position 571 is substituted with lysine (R484K_G571K), a modified enzyme in which the arginine at position 484 is substituted with lysine and the arginine at position 572 is substituted with lysine (R484K_R572K), a modified enzyme in which the arginine at position 484 is substituted with lysine and the arginine at position 572 is substituted with tryptophan (R484K_R572W), a modified enzyme in which the arginine at position 484 is substituted with lysine and the threonine at position 573 is substituted with lysine (R484K_T573K), a modified enzyme in which the arginine at position 484 is substituted with lysine and the threonine at position 573 is substituted with tryptophan (R484K_T573W), a modified enzyme in which the arginine at position 484 is substituted with lysine and the tyrosine at position 516 is substituted with lysine (R484K_Y516K), a modified enzyme in which the arginine at position 484 is substituted with tryptophan and the asparagine at position 515 is substituted with lysine (R484W_N515K), a modified enzyme in which the glycine at position 485 is substituted with aspartic acid and the lysine at position 487 is substituted with histidine (G485D_K487H), a modified enzyme in which the glycine at position 485 is substituted with lysine and the lysine at position 487 is substituted with histidine (G485K_K487H), a modified enzyme in which the glycine at position 485 is substituted with lysine and the glycine at position 569 is substituted with lysine (G485K_G569K), a modified enzyme in which the glycine at position 485 is substituted with lysine and the asparagine at position 514 is substituted with lysine (G485K_N514K), a modified enzyme in which the glycine at position 485 is substituted with lysine and the asparagine at position 515 is substituted with lysine (G485K_N515K), a modified enzyme in which the glycine at position 485 is substituted with lysine and the asparagine at position 515 is substituted with tryptophan (G485K_N515W), a modified enzyme in which the glycine at position 485 is substituted with arginine and the lysine at position 487 is substituted with aspartic acid (G485R_K487D), a modified enzyme in which the glycine at position 485 is substituted with tryptophan and the glutamic acid at position 513 is substituted with tryptophan (G485W_E513W), a modified enzyme in which the glycine at position 485 is substituted with tryptophan and the asparagine at position 515 is substituted with lysine (G485W_N515K), a modified enzyme in which the glycine at position 485 is substituted with tryptophan and the asparagine at position 515 is substituted with tryptophan (G485W_N515W), a modified enzyme in which the lysine at position 487 is substituted with alanine and the serine at position 531 is substituted with alanine (K487A_S531A), a modified enzyme in which the lysine at position 487 is substituted with alanine and the tryptophan at position 593 is substituted with phenylalanine (K487A_W593F), a modified enzyme in which the lysine at position 487 is substituted with alanine and the tyrosine at position 615 is substituted with leucine (K487A_Y615L), a modified enzyme in which the lysine at position 487 is substituted with tryptophan and the asparagine at position 514 is substituted with lysine (K487W_N514K), a modified enzyme in which the lysine at position 487 is substituted with tryptophan and the asparagine at position 514 is substituted with tryptophan (K487W_N514W), a modified enzyme in which the lysine at position 487 is substituted with tryptophan and the tyrosine at position 516 is substituted with tryptophan (K487W_Y516W), a modified enzyme in which the lysine at position 487 is substituted with tryptophan and the glycine at position 569 is substituted with lysine (K487W_G569K), a modified enzyme in which the lysine at position 487 is substituted with tyrosine and the asparagine at position 515 is substituted with phenylalanine (K487Y_N515F), a modified enzyme in which the lysine at position 487 is substituted with tyrosine and the asparagine at position 515 is substituted with tryptophan (K487Y_N515W), a modified enzyme in which the valine at position 488 is substituted with lysine and the asparagine at position 515 is substituted with lysine (V488K_N515K), a modified enzyme in which the valine at position 488 is substituted with lysine and the asparagine at position 515 is substituted with tryptophan (V488K_N515W), a modified enzyme in which the valine at position 488 is substituted with tryptophan and the asparagine at position 515 is substituted with lysine (V488W_N515K), a modified enzyme in which the valine at position 488 is substituted with tryptophan and the asparagine at position 515 is substituted with tryptophan (V488W_N515W), a modified enzyme in which the valine at position 488 is substituted with tyrosine and the asparagine at position 515 is substituted with tryptophan (V488Y N515W), a modified enzyme in which the asparagine at position 550 is substituted with serine and the tyrosine at position 598 is substituted with asparagine (N550S_Y598N), a modified enzyme in which the asparagine at position 550 is substituted with serine and the tyrosine at position 615 is substituted with glycine (N550S_Y615G), a modified enzyme in which the asparagine at position 550 is substituted with serine and the tyrosine at position 615 is substituted with leucine (N550S_Y615L), a modified enzyme in which the asparagine at position 550 is substituted with serine and the tyrosine at position 615 is substituted with proline (N550S_Y615P) (these modified enzymes meet the above-described condition 4 that “a variant enzyme produces oligosaccharides in which the content of trisaccharides and higher saccharides is approximately equal and the content of tetrasaccharides is higher, relative to the case of the parent wild-type enzyme”).

These specific examples represent modified enzymes in which two amino acids are substituted. On the other hand, examples of modified enzymes in which three or four amino acids are substituted can include a modified enzyme in which the arginine at position 484 is substituted with alanine, the glycine at position 485 is substituted with alanine, and the lysine at position 487 is substituted with aspartic acid (R484A_G485A_K487D), a modified enzyme in which the arginine at position 484 is substituted with aspartic acid, the glycine at position 485 is substituted with aspartic acid, and the lysine at position 487 is substituted with aspartic acid (R484D_G485D_K487D), a modified enzyme in which the glycine at position 485 is substituted with arginine, the aspartic acid at position 486 is substituted with arginine, and the lysine at position 487 is substituted with arginine (G485R_D486R_K487R), a modified enzyme in which the arginine at position 484 is substituted with alanine, the glycine at position 485 is substituted with alanine, the aspartic acid at position 486 is substituted with alanine, and the lysine at position 487 is substituted with alanine (R484A_G485A_D486A_K487A), a modified enzyme in which the arginine at position 484 is substituted with alanine, the glycine at position 485 is substituted with alanine, the aspartic acid at position 486 is substituted with proline, and the lysine at position 487 is substituted with proline (R484A_G485A_D486P_K487P) (these modified enzymes meet the above-described condition 3 that “a variant enzyme produces oligosaccharides in which the content of trisaccharides and higher saccharides is approximately equal and the content of tetrasaccharides is smaller, relative to the case of the parent wild-type enzyme”); a modified enzyme in which the arginine at position 484 is substituted with alanine, the glycine at position 485 is substituted with alanine, and the lysine at position 487 is substituted with aspartic acid (R484A_G485A_K487D), a modified enzyme in which the arginine at position 484 is substituted with cysteine, the aspartic acid at position 486 is substituted with histidine, and the lysine at position 487 is substituted with arginine (R484C_D486H_K487R), a modified enzyme in which the glycine at position 485 is substituted with arginine, the aspartic acid at position 486 is substituted with tryptophan, and the lysine at position 487 is substituted with tryptophan (G485R_D486W_K487W), a modified enzyme in which the glycine at position 485 is substituted with arginine, the aspartic acid at position 486 is substituted with alanine, and the lysine at position 487 is substituted with alanine (G485R_D486A_K487A), a modified enzyme in which the glycine at position 485 is substituted with arginine, the aspartic acid at position 486 is substituted with proline, and the lysine at position 487 is substituted with proline (G485R_D486P_K487P), a modified enzyme in which the arginine at position 484 is substituted with alanine, the glycine at position 485 is substituted with alanine, the aspartic acid at position 486 is substituted with alanine, and the lysine at position 487 is substituted with alanine (R484A_G485A_D486A_K487A), a modified enzyme in which the arginine at position 484 is substituted with alanine, the glycine at position 485 is substituted with alanine, the aspartic acid at position 486 is substituted with arginine, and the lysine at position 487 is substituted with arginine (R484A_G485A_D486R_K487R), a modified enzyme in which the arginine at position 484 is substituted with aspartic acid, the glycine at position 485 is substituted with aspartic acid, the aspartic acid at position 486 is substituted with arginine, and the lysine at position 487 is substituted with arginine (R484D_G485D_D486R_K487R), a modified enzyme in which the arginine at position 484 is substituted with aspartic acid, the glycine at position 485 is substituted with aspartic acid, the aspartic acid at position 486 is substituted with proline, and the lysine at position 487 is substituted with proline (R484D_G485D_D486P_K487P), a modified enzyme in which the arginine at position 484 is substituted with aspartic acid, the glycine at position 485 is substituted with aspartic acid, the aspartic acid at position 486 is substituted with alanine, and the lysine at position 487 is substituted with alanine (R484D_G485D_D486A_K487A), a modified enzyme in which the arginine at position 484 is substituted with aspartic acid, the glycine at position 485 is substituted with aspartic acid, the aspartic acid at position 486 is substituted with tryptophan, and the lysine at position 487 is substituted with tryptophan (R484D_G485D_D486W_K487W), a modified enzyme in which the arginine at position 484 is substituted with proline, the glycine at position 485 is substituted with proline, the aspartic acid at position 486 is substituted with arginine, and the lysine at position 487 is substituted with arginine (R484P_G485P_D486R_K487R) (these modified enzymes meet the above-described condition 4 that “a variant enzyme produces oligosaccharides in which the content of trisaccharides and higher saccharides is approximately equal and the content of tetrasaccharides is higher, relative to the case of the parent wild-type enzyme”).

The modified enzymes that have been described above are obtained by substituting a particular amino acid(s) of a reference β-galactosidase with other amino acid(s). Through the inventors' studies, there have been found, as useful modified enzymes other than variants with the above-described amino acid substitutions, modified enzymes in which an amino acid(s) is/are inserted at a particular position(s) in a given amino acid sequence (insertion variants), a particular amino acid(s) in a given amino acid sequence is/are deleted (deletion variants), and in addition, an amino acid substitution is applied to a deletion variant. Therefore, one embodiment of the present invention provides a modified enzyme which undergone any of the following modifications in a β-galactosidase consisting of the amino acid sequence of any of SEQ ID NOs. 1 to 4 or an amino acid sequence having 90% or more identity to the amino acid sequence set forth in any of SEQ ID NOs. 1 to 4:
(a) a deletion of the region of arginine at position 484 (R484) to lysine at position 487 (K487) or a region corresponding thereto;
(b) an insertion of an amino acid(s) at the position between asparagine at position 514 (N514) and asparagine at position 515 (N515) or at a position corresponding thereto;
(c) an insertion of an amino acid(s) at the position between proline at position 604 (P604) and tyrosine at position 605 (Y605) or at a position corresponding thereto;
(d) a deletion of the glycine at position 485 (G485) or an amino acid corresponding thereto, and in addition, a substitution of aspartic acid at position 486 (D486) or an amino acid corresponding thereto;
(e) a deletion of the region of glycine at position 485 (G485) to aspartic acid at position 486 (D486) or a region corresponding thereto, and in addition, a substitution of lysine at position 487 (K487) or an amino acid corresponding thereto;
(f) a deletion of the region of glycine at position 485 (G485) to aspartic acid at position 486 (D486) or a region corresponding thereto, and in addition, a substitution of arginine at position 484 (R484) or an amino acid corresponding thereto, and a substitution of lysine at position 487 (K487) or an amino acid corresponding thereto;
(g) an insertion of an amino acid(s) at the position between arginine at position 484 (R484) and glycine at position 485 (G485) or at a position corresponding thereto; and
(h) an insertion of an amino acid(s) at the position between glycine at position 485 (G485) and aspartic acid at position 486 (D486) or at a position corresponding thereto.

For (d) to (f), it is preferable that the amino acid that has been substituted for R484 is tryptophan or proline; for D486, histidine; and for K487, arginine, tryptophan, or proline. For (b), (c), (g), and (h), the number of amino acids to be inserted is, for example, from 1 to 10, preferably from 2 to 8. In the following are described specific examples of the above-described (a) to (g), along with their notation.

### <Notation for Modified Enzymes>

“N514-F-F-N515” refers to a modified enzyme having two F (phenylalanine) residues inserted between N514 and N515.

“Δ484-487” refers to a modified enzyme having a deletion of the amino acids at positions 484 to 487.

“Δ485-486_K487R” refers to a modified enzyme having both a deletion of the amino acids at positions 485 to 486 and a substitution of the amino acid at position 487 with R (arginine).

### Specific Examples of Modified Enzymes

(A) Modified enzymes meeting the above-described condition 3 that “a variant enzyme produces oligosaccharides in which the content of trisaccharides and higher saccharides is approximately equal and the content of tetrasaccharides is smaller, relative to the case of the parent wild-type enzyme,” which are represent by
Δ484-487,
N514-F-F-N515,
N514-W-W-N515,
P604-W-W-Y605,
P604-A-A-A-A-Y605, and
P604-F-F-F-F-Y605.
   (B) Modified enzymes meeting the above-described condition 4 that “a variant enzyme produces oligosaccharides in which the content of trisaccharides and higher saccharides is approximately equal and the content of tetrasaccharides is higher, relative to the case of the parent wild-type enzyme,” which are represent by
Δ485 D486H,
Δ485-486_K487R,
Δ485-486_R484W_K487W,
Δ485-486_R484W_K487P,
R484-A-A-G485,
R484-F-F-G485,
R484-K-K-G485,
R484-P-P-G485,
R484-R-R-G485,
R484-S-S-G485,
R484-W-W-G485,
G485-S-S-S-D486,
R484-A-A-A-A-G485,
R484-D-D-D-D-G485,
R484-F-F-F-F-G485,
R484-P-P-P-P-G485,
R484-R-R-R-R-G485,
R484-S-S-S-S-G485,
R484-W-W-W-W-G485,
R484-A-A-A-A-A-A-A-A-G485,
R484-D-D-D-D-D-D-D-D-G485,
R484-F-F-F-F-F-F-F-F-G485,
R484-K-K-K-K-K-K-K-K-G485, and
R484-P-P-P-P-P-P-P-P-G485.

As shown in the examples described below, other amino acid residues useful for alteration of the enzyme specificity were also found by an approach different from those by which the amino acids of groups 1 to 6 were identified. On the basis of the results from this approach, the present invention further provides the following modified β-galactosidase enzyme: a modified β-galactosidase in which, in addition to a substitution as described above, the tryptophan at position 1540 (W1540) is substituted with other amino acid in a β-galactosidase consisting of the amino acid sequence of SEQ ID NO. 1. The amino acid that has been substituted for the tryptophan preferably is alanine, cysteine, aspartic acid, glutamic acid, phenylalanine, glycine, histidine, isoleucine, lysine, leucine, methionine, asparagine, proline, glutamine, arginine, serine, threonine, or valine. Modified enzymes in this embodiment are highly useful in that, in addition to the modified enzyme having properties arising from an above-described substitution employed in combination, its use results in an increase in the content of tetrasaccharides.

From the results of further studies, as amino acids that are a target for substitution and that are highly useful in that the content of tetrasaccharides is increased in the case of a β-galactosidase consisting of the amino acid sequence of SEQ ID NO. 1, there were identified lysine at position 487 (K487), asparagine at position 514 (N514), and asparagine at position 515 (N515). The amino acids that have been substituted for the above-mentioned amino acids are preferably as indicated below.

The amino acid that has been substituted for lysine at position 487 (K487) is glycine or proline.

The amino acid that has been substituted for asparagine at position 514 (N514) is tryptophan.

The amino acid that has been substituted for asparagine at position 515 (N515) is lysine or tryptophan.

Besides, one of the β-galactosidases derived from Bacillus circulans is described in WO 2010/098561. It is also possible that a mutation corresponding to any of the amino acid substitutions disclosed in the present specification is applied to this β-galactosidase, thereby to obtain a modified β-galactosidase. The sequence coding β-galactosidase disclosed in WO 2010/098561 has about 70% identity to the sequence coding a reference β-galactosidase in the present application (SEQ ID NO. 5).

(Nucleic acid coding for modified β-galactosidase, etc.)

The second aspect of the present invention provides a nucleic acid relating to the modified enzyme of the invention. That is, provided are a gene coding for the modified enzyme, a nucleic acid that can be used as a probe for identifying a nucleic acid coding for the modified enzyme, and a nucleic acid that can be used as a primer for amplifying or mutating a nucleic acid coding for the modified enzyme.

The gene coding for a modified enzyme is typically used in preparation of the modified enzyme. According to a genetic engineering procedure using the gene coding for a modified enzyme, a modified enzyme in a more homogeneous state can be obtained. Further, the method can be a preferable method also in the case of preparing a large amount of a modified enzyme. Note that uses of the gene coding for a modified enzyme are not limited to preparation of a modified enzyme. For example, the nucleic acid can also be used as a tool for an experiment intended for clarification of action mechanisms of a modified enzyme or a tool for designing or preparing a further mutant of an enzyme.

The “gene coding for a modified enzyme” herein refers to a nucleic acid capable of obtaining the modified enzyme when it is expressed, and includes, as a matter of course of a nucleic acid having a nucleotide sequence corresponding to the amino acid sequence of the modified enzyme, also a nucleic acid obtained by adding a sequence that does not code for an amino acid sequence to such a nucleic acid. Degeneracy of a codon is also considered.

The nucleic acid of the present invention can be prepared in an isolated state by use of a standard genetic engineering technique, molecular biological technique, biochemical technique, and the like in reference to the present specification or the sequence information disclosed in the appended sequence listing.

Another embodiment of the present invention provides a nucleic acid different in a nucleotide sequence in a part (hereinafter also referred to as a “homologous nucleic acid”, and a nucleotide sequence defining a homologous nucleic acid is also referred to as a “homologous nucleotide sequence”) as compared to the nucleotide sequence of the gene coding for the modified enzyme of the invention, although functions of a protein coded by the nucleic acid are equal. An example of the homologous nucleic acid includes a DNA composed of a nucleotide sequence containing substitution, deletion, insertion, addition or inversion of 1 to several nucleotides on the basis of the nucleotide sequence of the nucleic acid coding for the modified enzyme of the present invention and coding for a protein having enzyme activity characteristic to the modified enzyme. Substitution or deletion of bases may occur in a plurality of sites. The “plurality” herein depends on positions or kinds of amino acid residues in a conformation of a protein coded by the nucleic acid but means, for example, 2 to 40 bases, preferably 2 to 20 bases, and more preferably 2 to 10 bases.

Such a homologous nucleic acid as described above can be obtained by, for example, a restriction enzyme treatment, a treatment with exonuclease, DNA ligase, etc., and introduction of mutation by a site-directed mutagenesis method (Molecular Cloning, Third Edition, Chapter 13, Cold Spring Harbor Laboratory Press, New York), and a random mutagenesis method (Molecular Cloning, Third Edition, Chapter 13, Cold Spring Harbor Laboratory Press, New York). The homologous nucleic acid can be obtained also in other methods such as exposure to ultraviolet radiation.

Another embodiment of the present invention relates to a nucleic acid having the complementary nucleotide sequence to the nucleotide sequence of the gene coding for the modified enzyme of the invention. Another embodiment of the present invention provides a nucleic acid having a nucleotide sequence with an identity of at least about 60%, 70%, 80%, 90%, 95%, 99%, or 99.9% to the nucleotide sequence of the gene coding for the modified enzyme of the invention or the complementary nucleotide sequence

Another embodiment of the present invention relates to a nucleic acid having a nucleotide sequence hybridizing to the complementary nucleotide sequence to the nucleotide sequence of the gene coding for the modified enzyme of the invention or its homologous nucleotide sequence under stringent conditions. The “stringent conditions” herein refer to conditions wherein a so-called specific hybrid is formed and a nonspecific hybrid is not formed. Such stringent conditions are known by a person skilled in the art and can be set in reference to, for example, Molecular Cloning (Third Edition, Cold Spring Harbor Laboratory Press, New York) and Current protocols in molecular biology (edited by Frederick M. Ausubel et al., 1987). Examples of the stringent conditions include conditions of using a hybridization liquid (50% formamide, 10×SSC (0.15 M NaCl, 15 mM sodium citrate, pH 7.0), a 5×Denhardt solution, 1% SDS, 10% dextran sulfate, 10 μg/ml of modified salmon sperm DNA, and a 50 mM phosphate buffer (pH7.5)) and incubating at about 42° C. to about 50° C., thereafter washing with 0.1×SSC and 0.1% SDS at about 65° C. to about 70° C. Examples of more preferable stringent conditions include conditions of using 50% formamide, 5×SSC (0.15 M NaCl, 15 mM sodium citrate, pH 7.0), a 1×Denhardt solution, 1% SDS, 10% dextran sulfate, 10 μg/ml of modified salmon sperm DNA, and a 50 mM phosphate buffer (pH 7.5) as a hybridization liquid.

Another embodiment of the present invention provides a nucleic acid (nucleic acid fragment) having a part of the nucleotide sequence of the gene coding for the modified enzyme of the invention or the complementary nucleotide sequence. Such a nucleic acid fragment can be used in detection, identification, and/or amplification of a nucleic acid having the nucleotide sequence of the gene coding for the modified enzyme of the present invention. For example, the nucleic acid fragment is designed so as to at least contain a part being hybridized to a sequential nucleotide moiety (for example, about 10 to about 100 nucleotides length, preferably about 20 to about 100 bases length, more preferably about 30 to about 100 bases length) in the nucleotide sequence of the gene coding for the modified enzyme of the invention. When used as a probe, the nucleic acid fragment can be labeled. Examples such as fluorescent substances, enzymes, and radioactive isotopes can be used for the labeling.

Another aspect of the present invention relates to a recombinant DNA containing the gene of the present invention (the gene coding for a modified enzyme). The recombinant DNA of the invention is provided in, for example, a form of a vector. The term “vector” in the present specification refers to a nucleic acid molecule that can transfer a nucleic acid inserted in the vector to a target such as a cell.

A suitable vector is selected according to its intended use (cloning, expression of a protein) and in consideration of a kind of a host cell. Examples include a M13 phage or an altered form thereof, a λ phage or an altered form thereof, and pBR322 or an altered form thereof (e.g., pB325, pAT153, pUC8), etc. as a vector having Escherichia coli as a host, pYepSec1, pMFa, and pYES2 as a vector having a yeast as a host, pAc, pVL, etc. as a vector having an insect cell as a host, and pCDM8, pMT2PC, etc. as a vector having a mammal cell as a host.

The vector of the present invention is preferably an expression vector. The “expression vector” refers to a vector capable of introducing a nucleic acid inserted in the expression vector into a target cell (host cell) and expressing it in the cell. The expression vector generally contains a promoter sequence necessary for expression of a nucleic acid inserted, an enhancer sequence for promoting expression, and the like. An expression vector containing a selective marker can also be used. When such an expression vector is used, presence or absence (and its degree) of introduction of the expression vector can be confirmed using a selective marker.

Insertion of the nucleic acid of the present invention into the vector, insertion of a selective marker gene (if necessary), insertion of a promoter (if necessary), and the like can be performed by using a standard recombinant DNA technique (for example, a known method of using a restriction enzyme and a DNA ligase, which can be referred in Molecular Cloning, Third Edition, 1.84, Cold Spring Harbor Laboratory Press, New York).

For the host cell, microorganisms such as Escherichia coli and budding yeasts (Saccharomyces cerevisiae) are preferably used from the viewpoint of easiness of handling, and host cells capable of duplicating a recombinant DNA and expressing a gene of a modified enzyme can be used. Examples of Escherichia coli include Escherichia coli BL21(DE3)pLysS in the case of using a T7 promoter, and Escherichia coli JM109 in other cases. Examples of budding yeasts include budding yeast SHY2, AH22, or INVScl (Invitrogen Ltd.).

Another aspect of the present invention relates to a microorganism having the recombinant DNA of the invention (that is, a transformant). The microorganism of the invention can be obtained by transfection or transformation using the vector of the invention described above. The transfection or transformation can be performed in, for example, the calcium chloride method (J. Mol. Biol., 53, 159 (1970)), the Hanahan method (J. Mol. Biol., 166, 557 (1983)), the SEM method (Gene, 96, 23 (1990)), a method by Chung, et al. (Proc. Natl. Acad. Sci. U.S.A. 86, 2172 (1989)), the calcium phosphate coprecipitation method, the electroporation method (Potter, H. et al., Proc. Natl. Acad. Sci. U.S.A. 81, 7161-7165 (1984)), and the lipofectin method (Felgner, P. L. et al., Proc. Natl. Acad. Sci. U.S.A. 84, 7413-7417 (1984)). Note that the microorganism of the present invention can be used in producing the modified enzyme of the present invention.

### (Enzyme Agent Containing Modified β-Galactosidase)

The modified enzyme of the present invention is provided, for example, in the form of an enzyme agent. The enzyme agent may contain an excipient, a buffer agent, a suspending agent, a stabilizer, a preservative, an antiseptic, saline and the like besides the active ingredient (the modified enzyme of the present invention). As the excipient, starch, dextrin, maltose, trehalose, lactose, D-glucose, sorbitol, D-mannitol, white soft sugar, glycerol and the like can be used. As the buffer agent, phosphates, citrates, acetates and the like can be used. As the stabilizer, propylene glycol, ascorbic acid and the like can be used. As the preservative, phenol, benzalkonium chloride, benzyl alcohol, chlorobutanol, methylparaben and the like can be used. As the antiseptic, ethanol, benzalkonium chloride, paraoxybenzoic acid, chlorobutanol and the like can be used.

### (Applications of Modified β-Galactosidases)

A further aspect of the present invention provides applications of modified enzymes or enzyme preparations. Examples of these applications include use for production of galacto-oligosaccharides, production and processing of pharmaceutical products and supplements for lactose-intolerant patients, production and processing of dairy products (for example, processed milk products such as lactose-reduced milk, powdered milk such as skim milk and nursing powdered milk, yogurts, and others), and production and processing of medical foods.

Modified enzymes of the present invention are particularly useful for the production of galacto-oligosaccharides. In the production of galacto-oligosaccharides, a predetermined amount (for example, 50 U to 1000 U) of a modified enzyme is added, for example, to a pre-heated lactose-dissolved solution (for example, 30% to 50% lactose, pH 7.0) and the mixture is left standing at around 40° C. for 1 to 10 hours, to yield galacto-oligosaccharides. Galacto-oligosaccharides are represented by the formula Gal-(Gal)n-Glc, wherein n is from 0 to about 5; and Gal is a galactose residue and Glc is a glucose residue. The manner in which Gal and Glc residues are bound includes not only μ1-6, β1-3, β1-4, β1-2, but also al-3, al-6, and others.

The use of a plurality of modified enzymes with different properties would make it possible that various galacto-oligosaccharides are produced separately as intended. This form of usage is effective in reproducing the profile of galacto-oligosaccharides (the type and ratio of galacto-oligosaccharides) contained in mother's milk.

A modified enzyme and the parent wild-type enzyme thereof are different in their properties. Therefore, the use of a modified enzyme and the parent wild-type enzyme thereof in combination makes it possible to manufacture a galacto-oligosaccharide of which the production cannot be achieved (or is not suitable) by using the wild-type enzyme alone. When a plurality of modified enzymes with different properties and the parent wild-type enzyme thereof are used in combination, it would be possible to further increase the kind of galacto-oligosaccharides to be produced. Thus, the use of (one or more) modified enzymes and the parent wild-type enzyme thereof in combination is also effective in producing various galacto-oligosaccharides separately as intended.

Methods by which galacto-oligosaccharides (a mixture of various types of galacto-oligosaccharides) are obtained using a plurality of modified enzymes (a combination of a plurality of modified enzymes, or alternatively a combination of a given wild-type enzyme and one or more modified enzymes thereof) include, when classified roughly, one in which galacto-oligosaccharides that have been produced using respective enzymes are mixed, one in which a plurality of enzymes is allowed to act on a raw material (lactose) at the same time, and one in which a plurality of enzymes is allowed to act on a raw material in a stepwise manner.

### EXAMPLES

### <Identification of Positions for Effective Mutations 1>

### 1. Purposes

A Bacillus circulans-derived β-galactosidase is an important enzyme for the industrial production of galacto-oligosaccharides. However, the galacto-oligosaccharides produced by using the enzyme are highly uniform in composition, and it is difficult that the composition (profile) of the galacto-oligosaccharides to be produced is altered even though the conditions under which they are produced are adjusted. In the past, it has been difficult to obtain the steric structure information of this enzyme, so that it has not been easy to identify amino acids responsible for its functions. For this reason, the inventors attempted to identify positions for effective mutations by means of using computer simulation, aiming at creating a modified enzyme useful for the production of galacto-oligosaccharides.

### 2. Methods and Results

### (1) Prediction of the Steric Structure of Wild-Type Enzymes and Design of Variant Enzymes (Modified β-Galactosidases)

### (1-1) Prediction of the Steric Structure and Design of Mutation Sites on the Basis of the Primary Sequences of the Wild-Type Enzymes

β-galactosidase derived from Bacillus circulans performs the hydrolysis of lactose and catalyze the trans-galactosylation reaction (production of galacto-oligosaccharides). From earlier studies, it is known that the β-galactosidase produced by Bacillus circulans is made up of four enzymes (isozymes) with different molecular weights, that is, β-galactosidase isozymes with a molecular weight of 195 kD (BgaD-A; SEQ ID NO. 1), 160 kD (BgaD-B; SEQ ID NO. 2), 135 kD (BgaD-C; SEQ ID NO. 3), and 86 kD (BgaD-D; SEQ ID NO. 4) (Patent Literature 1 and Non Patent Literature 1). Among these enzymes, BgaD-D with the lowest molecular weight is a β-galactosidase isozyme of the smallest size possessing enzymatic activity, and has the highest transglycosylation activity.

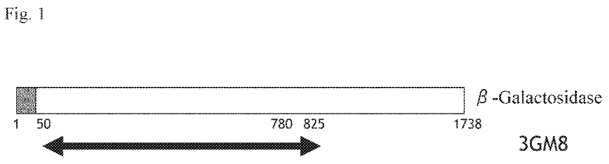

As described above, it has been difficult to obtain the steric structure information of β-galactosidase isozymes derived from Bacillus circulans. For this reason, the inventors attempted to predict their steric structures using computer simulation. Information on the steric structure of enzymes with β-galactosidase activity was extracted from the steric structures information deposited in a database (Protein Data Bank (http://www.pdb.org/)), thereby to search for enzymes having a sequence homologous to the primary sequence of any of the β-galactosidase isozymes derived from Bacillus circulans. Specifically, the primary sequence of a β-galactosidase isozyme derived from Bacillus circulans was used as a reference sequence to search for steric structures that are predicted to be highly homologous relative to the β-galactosidase isozyme. In consequence, there was found the structure 3GM8 (a beta-glycosidase from Bacteroides vulgatus) (FIG. 1). Subsequently, loop sequences located around the active center were identified based on a steric structure modeled using the 3GM8 as a template. By using the steric structure model thus constructed, amino acids that are likely to come into contact with the substrate were predicted from among the amino acids forming the substrate pocket of a β-galactosidase derived from Bacillus circulans. Furthermore, sites for introduction of mutation were designed, taking note of amino acids on the loops.

### (1-2) Construction of Plasmids and Generation of Variants

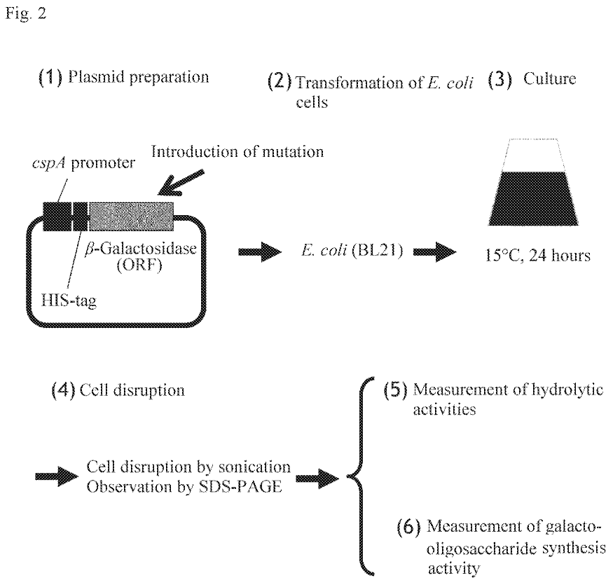

Amino acids at the sites where a mutation was to be introduced were intended to be subjected to random substitution. The actual introduction of mutations was performed via PCR using as a template a plasmid coding a DNA for BgaD type D. At a single site where a mutation was to be introduced, 8 to 19 amino acids to be substituted for the original amino acid were designed and used for actual mutations. Primers corresponding to the respective mutations were designed, and the respective mutations were introduced via an inverted PCR method using a KOD plus Mutagenesis kit (Toyobo Co., Ltd). A PCR product was self-ligated according to the protocol for the kit, and then transformed into E. coli DH5alpha cells. The plasmid was obtained by a mini-prep procedure and then sequenced to verify the mutation. In addition, the plasmid was used to transform into E. coli BL21 cells, which then were subjected to expression of the protein (variant) according to the protocol described below (FIG. 2).

### Protocol

1. Pre-culture in 1.5 mL of LB with amp (37° C., overnight)
2. Add 0.06 mL of the pre-culture to 3 mL of LB with amp
3. Incubate at 37° C. for 4 hours
4. Transfer the test tube on ice and add 0.75 μL of 1 M IPTG
5. Incubate at 15° C. for 24 hours
6. Harvest and wash cells with phosphate buffer (pH 7.4)
7. Suspend the cells in 0.25 mL phosphate buffer (pH 7.4)
8. Subject the cells to ultrasonic disruption in a sonicator (30 seconds×3 times)
9. Collect the supernatant by centrifugation

The crude E. coli cells extract prepared by the above procedure was used as a sample of the variant (modified β-galactosidase).

Further, variants were subjected to purification for verification of their activities. Each of the variants, which had been expressed as a fusion protein having a HIS tag attached at the N terminus, was purified on a nickel column HISTrap HP (1 ml; GE Healthcare). The buffers used for the respective operations were as follows:

A binding buffer (20 mM sodium phosphate, 0.2 M NaCl, 20 mM imidazole, pH 7.4)

An elution buffer (10 mM sodium phosphate, 0.1 M NaCl, 0.25 M imidazole, pH 7.4)

The variant was allowed to be bound on the column in the binding buffer, and then the column was washed with the same buffer. After that, the bound variant was eluted from the column in the elution buffer. The elution fractions were used as samples for SDS-PAGE and for measurements of hydrolytic activities, to ascertain the degree of purification of the enzyme.

### (1-3) Evaluation of Hydrolytic Activities

Measurement methods for hydrolysis were carried out with reference to a previous document (Patent Literature 1). As substrate, use was made of three substances, o-nitrophenyl-β-D-galactopyranoside (ONPG), lactose, and allolactose, for which the respective activities were measured by the methods described below.

### (a) LSU Activity: ONPG Method

When lactase acts on ONPG (at a final concentration of 20 mM) at pH 6.0 and 40° C., the amount of enzyme at which 1 μmol of ONPG is generated per minute in an early stage of the reaction is defined as 1 LSU.

### (b) LU Activity: Lactose Decomposition Assay

When lactase acts on lactose (at a final concentration of 10%) at pH 6.0 and 40° C., the amount of enzyme at which 1 μmol of glucose is generated per minute in an early stage of the reaction is defined as 1 LU.

### (c) Allolactose Decomposing Activity: Allolactose Decomposition Assay

When lactase acts on allolactose (at a final concentration of 1%) at pH 6.0 and 40° C., the amount of enzyme at which 1 μmol of glucose is generated per minute in an early stage of the reaction is defined as 1 U.

### (d) 1%-Lactose Decomposing Activity: Lactose Decomposition Assay

When lactase acts on lactose (at a final concentration of 1%) at pH 6.0 and 40° C., the amount of enzyme at which 1 μmol of glucose is generated per minute in an early stage of the reaction is defined as 1 U.

By these measurements, mutation sites were sought out which caused a change in the substrate specificity, as compared to that of the parent wild-type enzyme.

### (1-4) Evaluation of Galacto-Oligosaccharide (GOS) Synthesis Activity

For the evaluation of galacto-oligosaccharide (GOS) synthesis activity, an oligosaccharide synthesis reaction was carried out using a concentrated solution of lactose as the substrate, and reaction products were analyzed by HPLC. From earlier studies, it has been reported that when lactose is used as the substrate, a high-yield GOS synthesis can be done by carrying out the enzyme reaction at a final substrate concentration of about 50% and a pH of around 6 (JP H11-18763 A). The GOS synthesis was performed according to the method described in the report (the method described in Example 2). The composition of GOSs produced was analyzed using the following columns:
A gel filtration column: MCIGEL CK04S (Mitsubishi Kasei Corp.)
An amide column: NH2P-40 3E (Showa Denko K.K.)

On the basis of the analysis results, the contents of oligosaccharides for tetrasaccharides and for trisaccharides and higher saccharides, and of allolactose in the entire saccharides were determined to prepare tables.

### (2) Mutation Sites Resulting in a Decreased or Increased Hydrolytic Activity for Allolactose

The LSU activity, LU activity, allolactose-decomposing activity, and 1%-lactose decomposing activity were determined for recombinantly expressed enzymes of all variant enzymes and the parent wild-type enzymes thereof. First, from the results of measurements of the LSU activity and LU activity, variants with which the disappearance of enzymatic activity was observed were excluded. On the other hand, the allolactose decomposing activity varies depending upon the lactose decomposing activity, and thus values of “allolactose decomposing activity÷1%-lactose decomposing activity” were used to evaluate relative substrate specificities of variant enzymes for allolactose, which were compared to that of the parent wild-type enzyme.

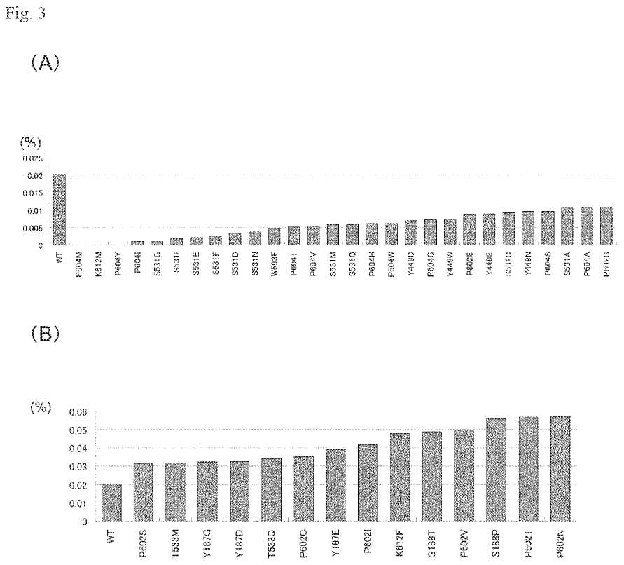

From the results of actual measurements, the parent wild-type enzyme was found to have a value of “allolactose decomposing activity÷1%-lactose decomposing activity” of 0.02%. On the basis of this observation, variants meeting either of the conditions described below were selected as variants having altered substrate specificity for allolactose (FIG. 3).

Condition 1: a variant enzyme has an allolactose decomposing activity of 50% or less relative to that of the parent wild-type enzyme, which means that the value of “allolactose decomposing activity÷1%-lactose decomposing activity” for a variant enzyme is not more than 1/2 of that for the parent wild-type enzyme.

Condition 2: a variant enzyme has an allolactose decomposing activity of 150% or more relative to that of the parent wild-type enzyme, which means that the value of “allolactose decomposing activity÷1%-lactose decomposing activity” for a variant enzyme is not less than 3/2 of that for the parent wild-type enzyme.

The variants meeting condition 1 were the following 28 variant enzymes (FIG. 3(A)), in which
the tyrosine at position 449 (Y449) was substituted with any of amino acids D, E, N, and W;
the serine at position 531 (S531) was substituted with any of amino acids A, D, E, F, G, I, M, N, and Q;
the tryptophan at position 593 (W593) was substituted with an amino acid F;
the proline at position 602 (P602) was substituted with any of amino acids E and G;
the proline at position 604 (P604) was substituted with any of amino acids A, G, H, I, M, S, T, V, W, and Y; and
the lysine at position 612 (K612) was substituted with an amino acid M.

The variants meeting condition 2 were the following 14 variant enzymes (FIG. 3(B)), in which
the tyrosine at position 187 (Y187) was substituted with any of amino acids D, E, and G;
the serine at position 188 (S188) was substituted with any of amino acids P and T;
the threonine at position 533 (T533) was substituted with any of amino acids M and Q;
the proline at position 602 (P602) was substituted with any of amino acids C, I, N, S, T, and V; and
the lysine at position 612 (K612) was substituted with an amino acid F.

From these results, it turned out that a total of 9 amino acids are related to the substrate specificity of the hydrolytic activity for allolactose.

### (3) Mutation Sites Resulting in a Change in the Composition of Galacto-Oligosaccharides (GOSs)

Mutation sites resulting in a change in the composition of galacto-oligosaccharides (GOSs) were evaluated for recombinantly expressed enzymes of all variant enzymes and the parent wild-type enzyme thereof. Variants meeting either of the conditions described below were selected as variants resulting in an altered composition of GOSs. First, the composition of GOSs produced was analyzed, and attention was paid to the contents of oligosaccharide components, tetrasaccharides, and trisaccharides and higher saccharides, in the GOSs produced. From the results of actual measurements, the parent wild-type enzyme was found to have a content of tetrasaccharides of 10.8% and a content of trisaccharides and higher saccharides of 40.0%. On the basis of this observation, mutation sites were selected in which the resultant variant enzymes gave a content of trisaccharides that was close to the value provided by the wild-type enzyme (that is, an amount of oligosaccharides synthesized that was comparable to that synthesized by the wild-type enzyme), but resulted in an altered content of tetrasaccharides.

Therefore, mutation sites were selected which meet either of the following conditions: Condition 3: a variant enzyme produces oligosaccharides in which the content of trisaccharides and higher saccharides is approximately equal and the content of tetrasaccharides is smaller, relative to the case of the parent wild-type enzyme.

Condition 4: a variant enzyme produces oligosaccharides in which the content of trisaccharides and higher saccharides is approximately equal and the content of tetrasaccharides is higher, relative to the case of the parent wild-type enzyme.

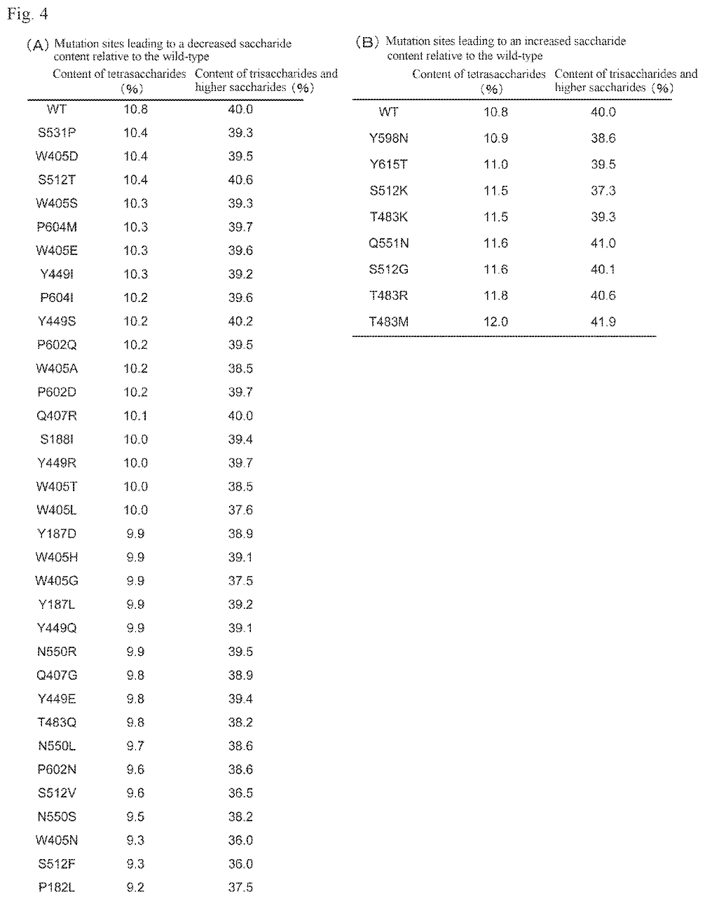

The variants meeting condition 3 were the following 33 variant enzymes (FIG. 4(A)), in which
the proline at position 182 (P182) was substituted with an amino acid L;
the tyrosine at position 187 (Y187) was substituted with any of amino acids D and L;
the serine at position 188 (S188) was substituted with an amino acid I;
the tryptophan at position 405 (W405) was substituted with any of amino acids A, D, E, G, H, L, N, S, and T;
the glutamine at position 407 (Q407) was substituted with any of amino acids G and R;
the tyrosine at position 449 (Y449) was substituted with any of amino acids E, I, Q, R, and S;
the threonine at position 483 (T483) was substituted with an amino acid Q;
the serine at position 512 (S512) was substituted with any of amino acids F, T and V;
the serine at position 531 (S531) was substituted with an amino acid P;
the asparagine at position 550 (N550) was substituted with any of amino acids L, R, and S;
the proline at position 602 (P602) was substituted with any of amino acids L, N, and Q; and
the proline at position 604 (P604) was substituted with any of amino acid I and M.

The variants meeting condition 4 were the following 8 variant enzymes (FIG. 4(B)), in which
the threonine at position 483 (T483) was substituted with any of amino acids K, M, and R;
the serine at position 512 (S512) was substituted with any of amino acids G and K;
the glutamine at position 551 (Q551) was substituted with an amino acid N;
the tyrosine at position 598 (Y598) was substituted with an amino acid N; and
the tyrosine at position 615 (Y615) was substituted with an amino acid T.

From these results, it turned out that a total of 9 amino acids are related to the amount of production of tetrasaccharides.

Second, attention was paid to the contents of allolactose and of trisaccharides and higher saccharides in the composition of GOSs produced. The parent wild-type enzyme was found to have a content of allolactose of 3.58% and a content of trisaccharides and higher saccharides of 40.2%. On the basis of this observation, sites for mutation were sought in which the resultant variant enzymes gave a content of trisaccharides that was close to the value provided by the wild-type enzyme, but resulted in an altered content of allolactose. Therefore, mutation sites were selected which meet either of the following conditions:

Condition 5: a variant enzyme produces oligosaccharides in which the content of trisaccharides and higher saccharides is approximately equal and the content of allolactose is smaller, relative to the case of the parent wild-type enzyme.

Condition 6: a variant enzyme produces oligosaccharides in which the content of trisaccharides and higher saccharides is approximately equal and the content of allolactose is higher, relative to the case of the parent wild-type enzyme.

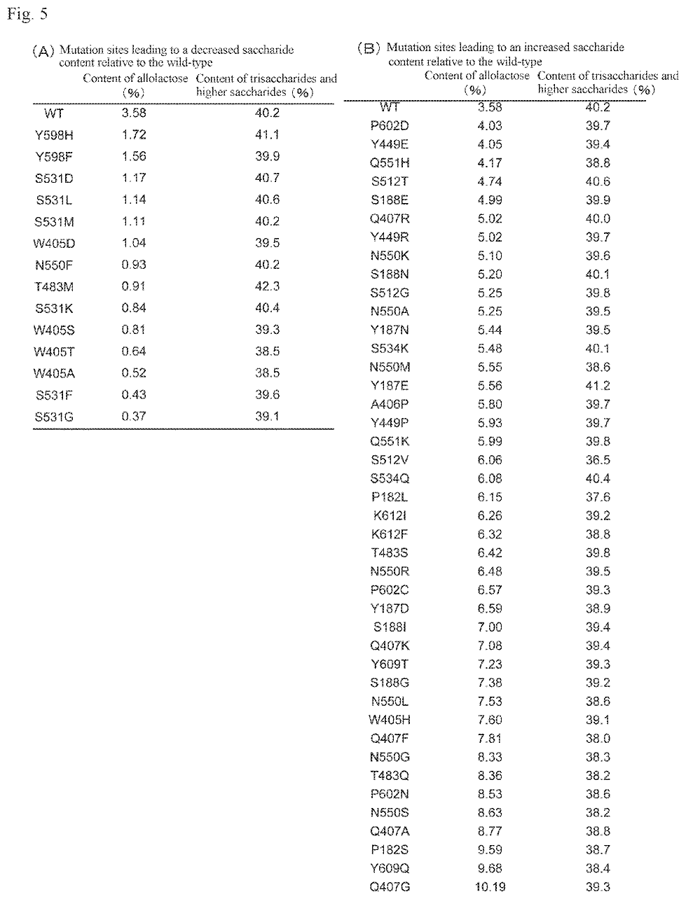

The variants meeting condition 5 were the following 14 variant enzymes (FIG. 5(A)), in which
the tryptophan at position 405 (W405) was substituted with any of amino acids A, D, S, and T;
the threonine at position 483 (T483) was substituted with an amino acid M;
the serine at position 531 (S531) was substituted with any of amino acids D, F, G, L, K, and M;
the asparagine at position 550 (N550) was substituted with an amino acid F; and
the tyrosine at position 598 (Y598) was substituted with any of amino acids F and H.

The variants meeting condition 6 were the following 45 variant enzymes (FIG. 5(B)), in which
the proline at position 182 (P182) was substituted with any of amino acids L and S;
the tyrosine at position 187 (Y187) was substituted with any of amino acids D, E, and N;
the serine at position 188 (S188) was substituted with any of amino acids E, G, I, and N;
the tryptophan at position 405 (W405) was substituted with an amino acid H;
the alanine at position 406 (A406) was substituted with an amino acid P;
the glutamine at position 407 (Q407) was substituted with any of amino acids A, F, G, K and R;
the tyrosine at position 449 (Y449) was substituted with any of amino acids E and R;
the threonine at position 483 (T483) was substituted with any of amino acids S and Q;
the serine at position 512 (S512) was substituted with any of amino acids G, T and V;
the serine at position 534 (S534) was substituted with any of amino acids K and Q;
the asparagine at position 550 (N550) was substituted with any of amino acids A, G, K, L, M, R, and S;
the glutamine at position 551 (Q551) was substituted with any of amino acids K and H;
the proline at position 602 (P602) was substituted with any of amino acids C, D, and N;
the tyrosine at position 609 (Y609) was substituted with any of amino acids D, Q, and T;
the lysine at position 612 (K612) was substituted with any of amino acids F and I; and
the tyrosine at position 615 (Y615) was substituted with any of amino acids E, I, and T.

From these results, it turned out that a total of 19 amino acids are related to the content of allolactose.

### (4) Mutation Sites in BgaD Type A

In the procedures shown in the above section (1-1), the designing of applying mutation to the loop structures located around the active center was made. On the other hand, the amino acids related to GOS synthesis has been reported for BgaD type A. In particular, it was reported that the substitution of the tryptophan at position 1540 (W1540) with alanine or phenylalanine led to a higher maximum yield of oligosaccharide synthesis (Non Patent Literature 1). Taking note of this report, the inventors assumed that the composition of GOSs to be produced could be altered by applying a mutation not only to an amino acid located around the active center that have been examined, but also to an amino acid present only in BgaD type A. On the basis of this assumption, the inventors actually attempted to perform saturation mutagenesis, in which the tryptophan at position 1540 (W1540) was substituted with 19 amino acids. In particular, W1540 was substituted with the other natural amino acids in a similar procedure as in the section (1-2), using as a template a plasmid coding a DNA for BgaD type A (SEQ ID NO. 5). In addition, the plasmid constructed was introduced into E. coli cells to express a variant enzyme. Furthermore, the GOS synthesis reaction was carried out in a similar way as in the section (1-4), and the contents of oligosaccharide components, tetrasaccharides and trisaccharides and higher saccharides, in the composition of GOSs produced were analyzed.

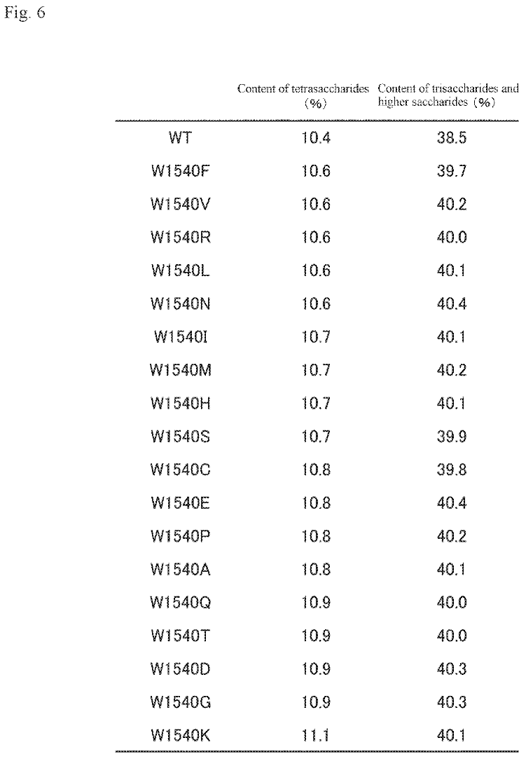

From the results of measurements of the composition of GOSs produced, the parent wild-type enzyme was found to have a content of tetrasaccharides of 10.4% and a content of trisaccharides and higher saccharides of 38.5%. On the basis of this observation, examination was made as to whether a change in the content of tetrasaccharides was observed in all the variants. From the results, it was found that 18 variant enzymes resulted in a higher content of tetrasaccharides than the wild-type enzyme (FIG. 6). Specifically, it turned out that the amount of production of tetrasaccharides was increased in the case of the following variants, in which
the tryptophan at position 1540 (W1540) was substituted with any of amino acids A, C, D, E, F, G, H, I, K, L, M, N, P, Q, R, S, T, and V.

From the results described above, it turned out that an effect of increasing the amount of production of tetrasaccharides was observed by applying mutation not only to the loop structures around the active center, but also to the amino acid at position 1540.

### 3. Summary

(1) Variants were found to have a decreased or increased hydrolytic activity for saccharides with a β-1,6-bond (mutations at 9 amino acid sites yielded 42 variants).

(2) The galacto-oligosaccharides produced by variants had altered degrees of polymerization and percentages of branched chains (mutation sites leading to an altered content of tetrasaccharides: 15 amino acid sites, yielding 41 variants; mutation sites leading to an altered percentage of branched chains: 19 amino acid sites, yielding 49 variants).

(3) Although the parent wild-type enzyme was capable of producing only oligosaccharides with a uniform composition, the use of variants would make it possible to produce various types of galacto-oligosaccharides separately as intended. Variants can be used, for example, for reproducing the components of mother's milk.

(4) It will be possible to construct even more useful variants by combining mutation sites that have been found to be effective by the above investigation.

### <Identification of Positions for Effective Mutations 2>

From the results of further studies, there were further identified positions for effective mutations, whereby useful variants were obtained. In the following, the variants (variant enzymes) that were successfully obtained and their characteristics are described. Here, the variants in (A), (B), and (D) to (N) are derived from BgaD type D β-galactosidase (SEQ ID NO. 4), whereas the variants in (C) are derived from BgaD type A β-galactosidase (SEQ ID NO. 1).

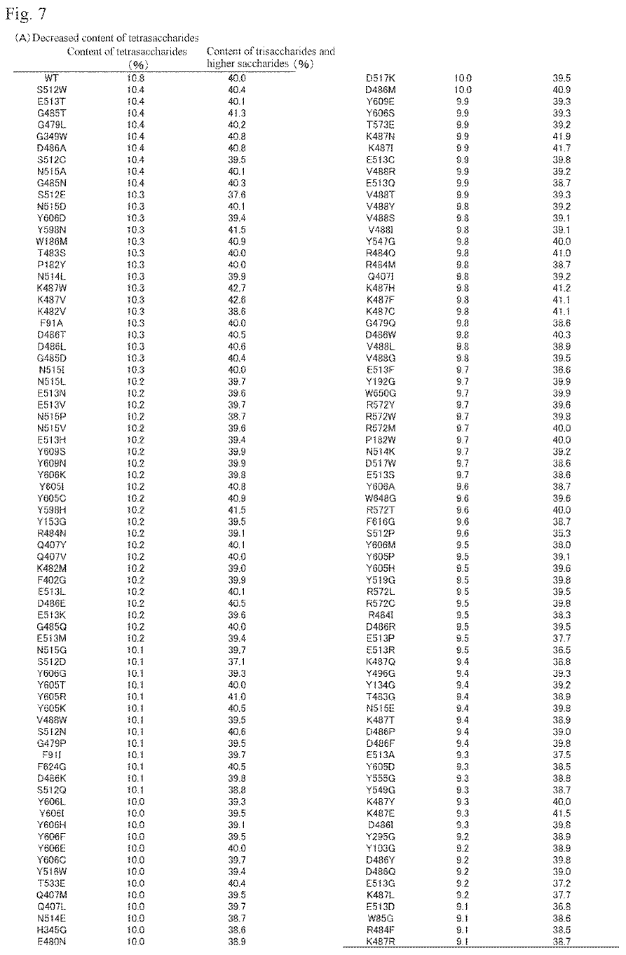

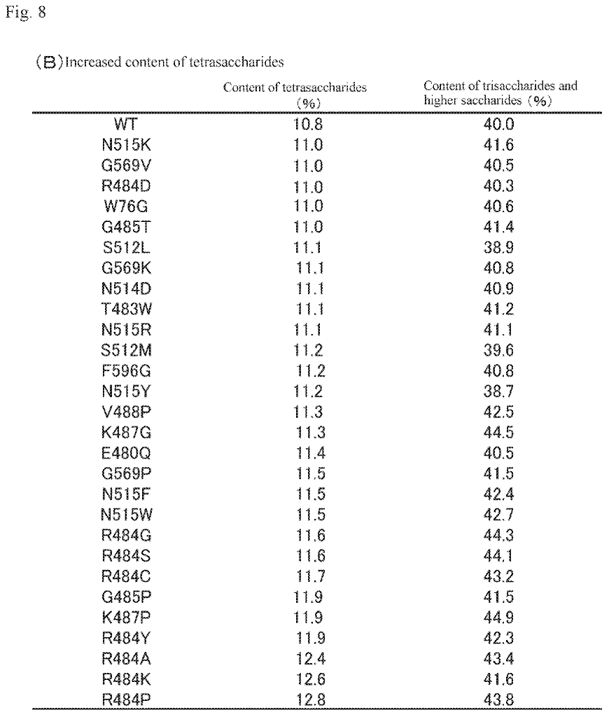

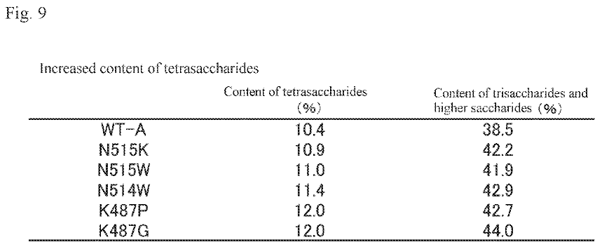

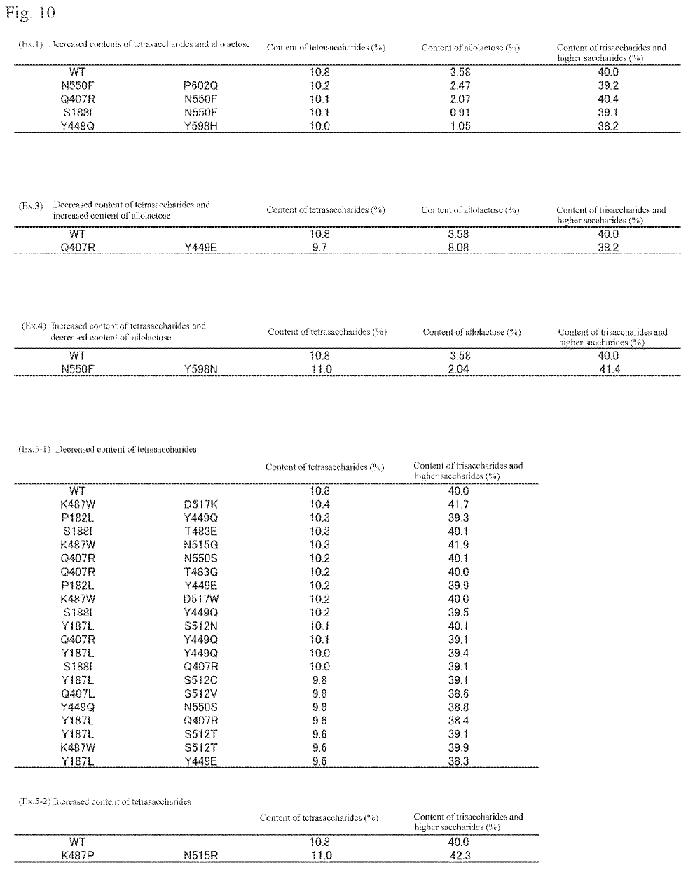

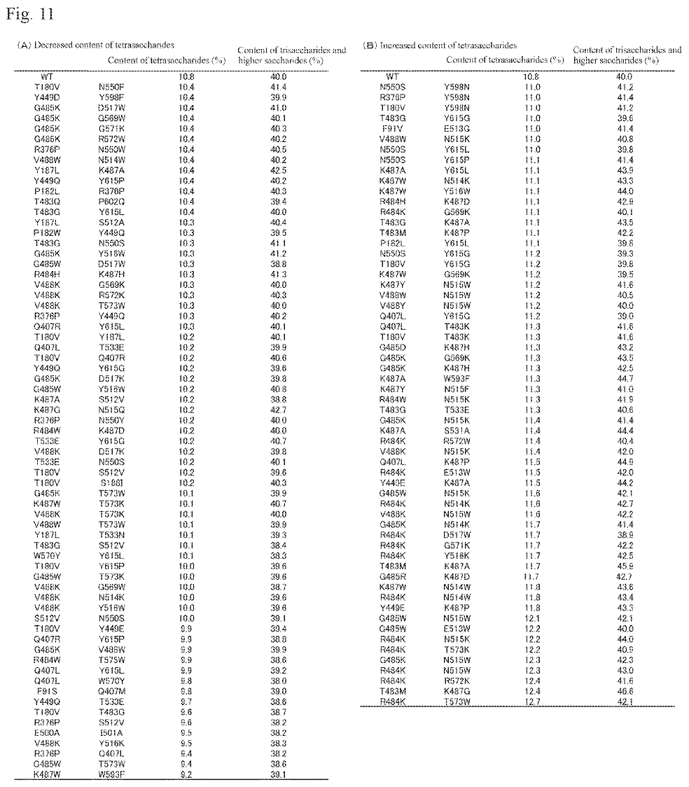

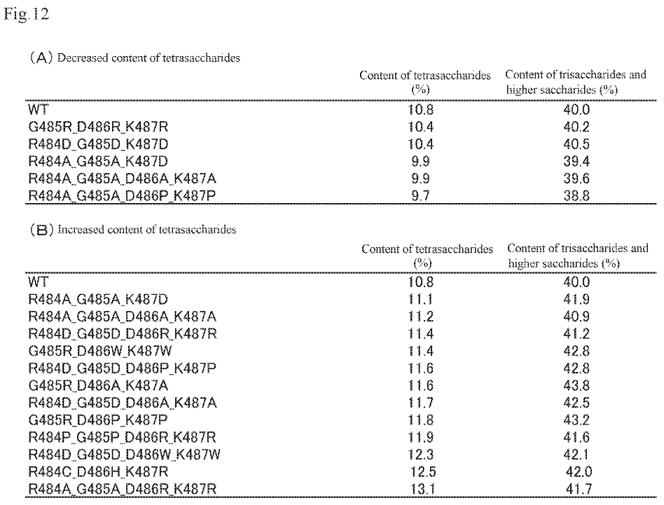

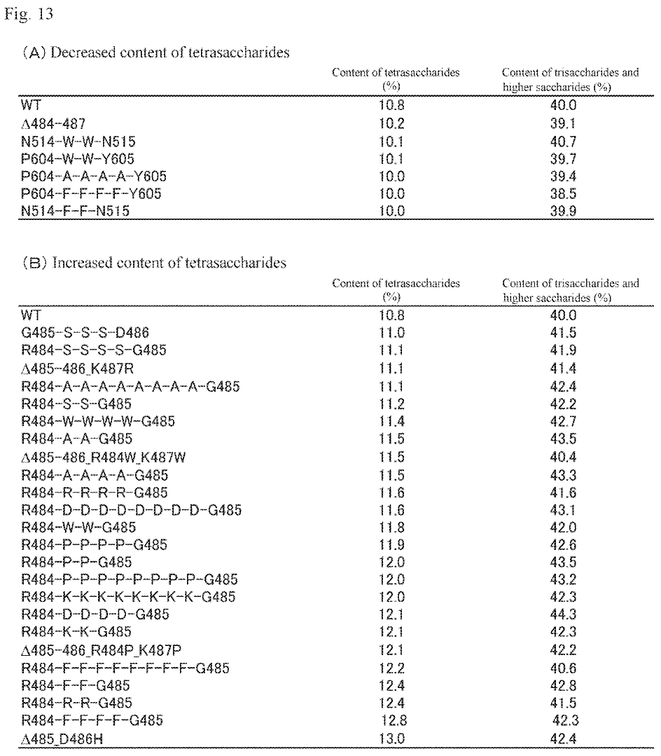

(A) Variants meeting condition 3 and having undergone a single amino acid substitution (FIG. 7), in which
the tryptophan at position 85 (W85) was substituted with glycine;
the phenylalanine at position 91 (F91) was substituted with alanine or isoleucine;
the tyrosine at position 103 (Y103) was substituted with glycine;
the tyrosine at position 134 (Y134) was substituted with glycine;
the tyrosine at position 153 (Y153) was substituted with glycine;
the proline at position 182 (P182) was substituted with tryptophan or tyrosine;
the tryptophan at position 186 (W186) was substituted with methionine;
the tyrosine at position 192 (Y192) was substituted with glycine;
the tyrosine at position 295 (Y295) was substituted with glycine;
the histidine at position 345 (H345) was substituted with glycine;
the glycine at position 349 (G349) was substituted with tryptophan;
the phenylalanine at position 402 (F402) was substituted with glycine;
the glutamine at position 407 (Q407) was substituted with isoleucine, leucine, methionine, valine, or tyrosine;
the glycine at position 479 (G479) was substituted with leucine, proline, or glutamine;
the glutamic acid at position 480 (E480) was substituted with asparagine;
the lysine at position 482 (K482) was substituted with methionine or valine;
the threonine at position 483 (T483) was substituted with glycine or serine;
the arginine at position 484 (R484) was substituted with phenylalanine, isoleucine, methionine, asparagine, or glutamine;
the glycine at position 485 (G485) was substituted with aspartic acid, asparagine, glutamine, or threonine;
the aspartic acid at position 486 (D486) was substituted with alanine, glutamic acid, phenylalanine, isoleucine, lysine, leucine, methionine, proline, glutamine, arginine, threonine, tryptophan, or tyrosine;
the lysine at position 487 (K487) was substituted with cysteine, glutamic acid, phenylalanine, histidine, isoleucine, leucine, asparagine, glutamine, arginine, threonine, valine, tryptophan, or tyrosine;
the valine at position 488 (V488) was substituted with glycine, isoleucine, leucine, arginine, serine, threonine, tryptophan, or tyrosine;
the tyrosine at position 496 (Y496) was substituted with glycine;
the serine at position 512 (S512) was substituted with cysteine, aspartic acid, glutamic acid, asparagine, proline, glutamine, or tryptophan;
the glutamic acid at position 513 (E513) was substituted with alanine, cysteine, aspartic acid, phenylalanine, glycine, histidine, lysine, leucine, methionine, asparagine, proline, glutamine, arginine, serine, threonine, or valine;
the asparagine at position 514 (N514) was substituted with glutamic acid, leucine, or lysine;
the asparagine at position 515 (N515) was substituted with alanine, aspartic acid, glutamic acid, glycine, isoleucine, leucine, proline, or valine;
the tyrosine at position 516 (Y516) was substituted with tryptophan;
the aspartic acid at position 517 (D517) was substituted with lysine or tryptophan;
the tyrosine at position 519 (Y519) was substituted with glycine;
the threonine at position 533 (T533) was substituted with glutamic acid;
the tyrosine at position 547 (Y547) was substituted with glycine;
the tyrosine at position 549 (Y549) was substituted with glycine;
the tyrosine at position 555 (Y555) was substituted with glycine;
the arginine at position 572 (R572) was substituted with cysteine, leucine, methionine, threonine, tryptophan, or tyrosine;
the threonine at position 573 (T573) was substituted with glutamic acid;
the tyrosine at position 598 (Y598) was substituted with histidine or asparagine;
the tyrosine at position 605 (Y605) was substituted with cysteine, aspartic acid, histidine, isoleucine, lysine, proline, arginine, or threonine;
the tyrosine at position 606 (Y606) was substituted with alanine, cysteine, aspartic acid, glutamic acid, phenylalanine, glycine, histidine, isoleucine, lysine, leucine, methionine, or serine;
the tyrosine at position 609 (Y609) was substituted with glutamic acid, asparagine, or serine;
the phenylalanine at position 616 (F616) was substituted with glycine;
the phenylalanine at position 624 (F624) was substituted with glycine;
the tryptophan at position 648 (W648) was substituted with glycine; and
the tryptophan at position 650 (W650) was substituted with glycine.
   (B) Variants meeting condition 4 and having undergone a single amino acid substitution (FIG. 8), in which
the tryptophan at position 76 (W76) was substituted with glycine;
the glutamic acid at position 480 (E480) was substituted with glutamine;
the threonine at position 483 (T483) was substituted with tryptophan;
the glycine at position 485 (G485) was substituted with proline or threonine;
the arginine at position 484 (R484) was substituted with alanine, cysteine, aspartic acid, glycine, proline, serine, tyrosine, or lysine;
the lysine at position 487 (K487) was substituted with glycine or proline;
the valine at position 488 (V488) was substituted with proline;
the serine at position 512 (S512) was substituted with leucine or methionine;
the asparagine at position 514 (N514) was substituted with aspartic acid;
the asparagine at position 515 (N515) was substituted with phenylalanine, arginine, tryptophan, tyrosine, or lysine;
the glycine at position 569 (G569) was substituted with lysine, proline or valine; and
the phenylalanine at position 596 (F596) was substituted with glycine.
   (C) Variants having an increased content of tetrasaccharides and having undergone a single amino acid substitution (variants derived from BgaD type A β-galactosidase) (FIG. 9), in which
the lysine at position 487 (K487) was substituted with glycine or proline;
the asparagine at position 514 (N514) was substituted with tryptophan; and
the asparagine at position 515 (N515) was substituted with lysine or tryptophan.
   (D) Variants having a combination of a substitution meeting condition 3 and a substitution meeting condition 5 (FIG. 10), in which
the serine at position 188 was substituted with isoleucine and the asparagine at position 550 was substituted with phenylalanine (S188I _N550F);
the glutamine at position 407 was substituted with arginine and the asparagine at position 550 was substituted with phenylalanine (Q407R_N550F);
the tyrosine at position 449 was substituted with glutamine and the tyrosine at position 598 was substituted with histidine (Y449Q_Y598H); and
the asparagine at position 550 was substituted with phenylalanine and the proline at position 602 was substituted with glutamine (N550F_P602Q).
   (E) A variant having a combination of a substitution meeting condition 3 and a substitution meeting condition 6 (FIG. 10), in which
the glutamine at position 407 was substituted with arginine and the tyrosine at position 449 was substituted with glutamic acid (Q407R_Y449E).
   (F) A variant having a combination of a substitution meeting condition 4 and a substitution meeting condition 5 (FIG. 10), in which
the asparagine at position 550 was substituted with phenylalanine and the tyrosine at position 598 was substituted with asparagine (N550F_Y598N).
   (G) Variants having a combination of substitutions meeting condition 3 (FIG. 10), in which
the proline at position 182 was substituted with leucine and the tyrosine at position 449 was substituted with glutamic acid (P182L_Y449E);
the proline at position 182 was substituted with leucine and the tyrosine at position 449 was substituted with glutamine (P182L_Y449Q);
the tyrosine at position 187 was substituted with leucine and the glutamine at position 407 was substituted with arginine (Y187L_Q407R);
the tyrosine at position 187 was substituted with leucine and the tyrosine at position 449 was substituted with glutamic acid (Y187L_Y449E);
the tyrosine at position 187 was substituted with leucine and the tyrosine at position 449 was substituted with glutamine (Y187L_Y449Q);
the tyrosine at position 187 was substituted with leucine and the serine at position 512 was substituted with cysteine (Y187L_S512C);
the tyrosine at position 187 was substituted with leucine and the serine at position 512 was substituted with asparagine (Y187L_S512N);
the tyrosine at position 187 was substituted with leucine and the serine at position 512 was substituted with threonine (Y187L_S512T);
the serine at position 188 was substituted with isoleucine and the glutamine at position 407 was substituted with arginine (S188I_Q407R);
the serine at position 188 was substituted with isoleucine and the tyrosine at position 449 was substituted with glutamine (S188I_Y449Q);
the serine at position 188 was substituted with isoleucine and the threonine at position 483 was substituted with glutamic acid (S188I_T483E);
the glutamine at position 407 was substituted with leucine and the serine at position 512 was substituted with valine (Q407L_S512V);
the glutamine at position 407 was substituted with arginine and the threonine at position 483 was substituted with glycine (Q407R_T483G);
the glutamine at position 407 was substituted with arginine and the tyrosine at position 449 was substituted with glutamine(Q407R_Y449Q);
the glutamine at position 407 was substituted with arginine and the asparagine at position 550 was substituted with serine (Q407R_N550S);
the tyrosine at position 449 was substituted with glutamine and the asparagine at position 550 was substituted with serine (Y449Q_N550S);
the lysine at position 487 was substituted with tryptophan and the serine at position 512 was substituted with threonine (K487W_S512T);
the lysine at position 487 was substituted with tryptophan and the aspartic acid at position 517 was substituted with lysine (K487W_D517K);
the lysine at position 487 was substituted with tryptophan and the asparagine at position 515 was substituted with glycine (K487W_N515G); and
the lysine at position 487 was substituted with tryptophan and the aspartic acid at position 517 was substituted with tryptophan (K487W_D517W).
   (H) A variant having a combination of substitutions meeting condition 4 (FIG. 10), in which the lysine at position 487 was substituted with proline and the asparagine at position 515 was substituted with arginine (K487P N515R).
   (I) Variants meeting condition 3 and having undergone two amino acid substitutions (FIG. 11), in which
the phenylalanine at position 91 was substituted with serine and the glutamine at position 407 was substituted with methionine (F91S_Q407M);
the threonine at position 180 was substituted with valine and the tyrosine at position 187 was substituted with leucine (T180V_Y187L);
the threonine at position 180 was substituted with valine and the serine at position 188 was substituted with isoleucine (T180V_S188I);
the threonine at position 180 was substituted with valine and the glutamine at position 407 was substituted with arginine (T180V_Q407R);
the threonine at position 180 was substituted with valine and the tyrosine at position 449 was substituted with glutamic acid (T180V_Y449E);
the threonine at position 180 was substituted with valine and the threonine at position 483 was substituted with glycine (T180V_T483G);
the threonine at position 180 was substituted with valine and the serine at position 512 was substituted with valine (T180V_S512V);
the threonine at position 180 was substituted with valine and the asparagine at position 550 is substituted with phenylalanine (T180V_N550F);
the threonine at position 180 was substituted with valine and the tyrosine at position 615 was substituted with proline (T180V_Y615P);
the proline at position 182 was substituted with leucine and the arginine at position 376 was substituted with proline (P182L_R376P);
the proline at position 182 was substituted with tryptophan and the tyrosine at position 449 was substituted with glutamine (P182W_Y449Q);
the tyrosine at position 187 was substituted with leucine and the lysine at position 487 was substituted with alanine (Y187L_K487A);
the tyrosine at position 187 was substituted with leucine and the serine at position 512 was substituted with alanine (Y187L_S512A);
the tyrosine at position 187 was substituted with leucine and the threonine at position 533 was substituted with asparagine (Y187L_T533N);
the arginine at position 376 was substituted with proline and the glutamine at position 407 was substituted with leucine (R376P_Q407L);
the arginine at position 376 was substituted with proline and the tyrosine at position 449 was substituted with glutamine (R376P_Y449Q);
the arginine at position 376 was substituted with proline and the serine at position 512 was substituted with valine (R376P_S512V);
the arginine at position 376 was substituted with proline and the asparagine at position 550 was substituted with tryptophan (R376P_N550W);
the arginine at position 376 was substituted with proline and the asparagine at position 550 was substituted with tyrosine (R376P_N550Y);
the glutamine at position 407 was substituted with leucine and the threonine at position 533 was substituted with glutamic acid (Q407L_T533E);
the glutamine at position 407 was substituted with leucine and the tryptophan at position 570 was substituted with tyrosine (Q407L_W570Y);
the glutamine at position 407 was substituted with leucine and the tyrosine at position 615 was substituted with leucine (Q407L_Y615L);
the glutamine at position 407 was substituted with arginine and the tyrosine at position 615 was substituted with leucine (Q407R_Y615L);
the glutamine at position 407 was substituted with arginine and the tyrosine at position 615 was substituted with proline (Q407R_Y615P);
the tyrosine at position 449 was substituted with aspartic acid and the tyrosine at position 598 was substituted with phenylalanine (Y449D_Y598F);
the tyrosine at position 449 was substituted with glutamine and the threonine at position 533 was substituted with glutamic acid (Y449Q_T533E);
the tyrosine at position 449 was substituted with glutamine and the tyrosine at position 615 was substituted with glycine (Y449Q_Y615G);
the tyrosine at position 449 was substituted with glutamine and the tyrosine at position 615 was substituted with proline (Y449Q_Y615P);
the threonine at position 483 was substituted with glycine and the serine at position 512 was substituted with valine (T483G_S512V);
the threonine at position 483 was substituted with glycine and the asparagine at position 550 was substituted with serine (T483G_N550S);
the threonine at position 483 was substituted with glycine and the tyrosine at position 615 was substituted with leucine (T483G_Y615L);
the threonine at position 483 was substituted with glutamine and the proline at position 602 was substituted with glutamine (T483Q_P602Q);
the arginine at position 484 was substituted with histidine and the lysine at position 487 was substituted with histidine (R484H_K487H);
the arginine at position 484 was substituted with tryptophan and the lysine at position 487 was substituted with aspartic acid (R484W_K487D);
the arginine at position 484 was substituted with tryptophan and the threonine at position 575 is substituted with tryptophan (R484W_T575W);
the glycine at position 485 was substituted with lysine and the valine at position 488 was substituted with tryptophan (G485K_V488W);
the glycine at position 485 was substituted with lysine and the tyrosine at position 516 was substituted with tryptophan (G485K_Y516W);
the glycine at position 485 was substituted with lysine and the aspartic acid at position 517 was substituted with lysine (G485K_D517K);
the glycine at position 485 was substituted with lysine and the aspartic acid at position 517 was substituted with tryptophan (G485K_D517W);
the glycine at position 485 was substituted with lysine and the glycine at position 569 was substituted with tryptophan (G485K_G569W);
the glycine at position 485 was substituted with lysine and the glycine at position 571 was substituted with lysine (G485K_G571K);
the glycine at position 485 was substituted with lysine and the arginine at position 572 was substituted with tryptophan (G485K_R572W);
the glycine at position 485 was substituted with lysine and the threonine at position 573 was substituted with tryptophan (G485K_T573W);
the glycine at position 485 was substituted with tryptophan and the tyrosine at position 516 was substituted with tryptophan (G485W_Y516W);
the glycine at position 485 was substituted with tryptophan and the aspartic acid at position 517 was substituted with tryptophan (G485W_D517W);
the glycine at position 485 was substituted with tryptophan and the threonine at position 573 is substituted with lysine (G485W_T573K);
the glycine at position 485 was substituted with tryptophan and the threonine at position 573 was substituted with tryptophan (G485W_T573W);
the lysine at position 487 was substituted with alanine and the serine at position 512 was substituted with valine (K487A_S512V);
the lysine at position 487 was substituted with glycine and the asparagine at position 515 was substituted with glutamine (K487G N515Q);
the lysine at position 487 was substituted with tryptophan and the threonine at position 573 was substituted with lysine (K487W_T573K);
the lysine at position 487 was substituted with tryptophan and the tryptophan at position 593 was substituted with phenylalanine (K487W_W593F);
the valine at position 488 was substituted with lysine and the asparagine at position 514 was substituted with lysine (V488K_N514K);
the valine at position 488 was substituted with lysine and the aspartic acid at position 517 was substituted with lysine (V488K_D517K);
the valine at position 488 was substituted with lysine and the glycine at position 569 was substituted with lysine (V488K_G569K);
the valine at position 488 was substituted with lysine and the glycine at position 569 was substituted with tryptophan (V488K_G569W);
the valine at position 488 was substituted with lysine and the arginine at position 572 was substituted with lysine (V488K_R572K);
the valine at position 488 was substituted with lysine and the threonine at position 573 was substituted with lysine (V488K_T573K);
the valine at position 488 was substituted with lysine and the threonine at position 573 was substituted with tryptophan (V488K_T573W);
the valine at position 488 was substituted with lysine and the tyrosine at position 516 was substituted with lysine (V488K_Y516K);
the valine at position 488 was substituted with lysine and the tyrosine at position 516 was substituted with tryptophan (V488K_Y516W);
the valine at position 488 was substituted with tryptophan and the asparagine at position 514 was substituted with tryptophan (V488W N514W);
the valine at position 488 was substituted with tryptophan and the threonine at position 573 is substituted with tryptophan (V488W_T573W);
the glutamic acid at position 500 was substituted with alanine and the isoleucine at position 501 was substituted with alanine (E500A_I501A);
the serine at position 512 was substituted with valine and the asparagine at position 550 was substituted with serine (S512V_N550S);
the threonine at position 533 was substituted with glutamic acid and the asparagine at position 550 was substituted with serine (T533E_N550S);
the threonine at position 533 was substituted with glutamic acid and the tyrosine at position 615 was substituted with glycine (T533E_Y615G); and
the tryptophan at position 570 was substituted with tyrosine and the tyrosine at position 615 was substituted with leucine (W570Y_Y615L).
   (J) Variants meeting condition 4 and having undergone two amino acid substitutions (FIG. 11), in which
the phenylalanine at position 91 was substituted with valine and the glutamic acid at position 513 was substituted with glycine (F91V_E513G);
the threonine at position 180 was substituted with valine and the threonine at position 483 was substituted with lysine (T180V_T483K);
the threonine at position 180 was substituted with valine and the tyrosine at position 598 was substituted with asparagine (T180V_Y598N);
the threonine at position 180 was substituted with valine and the tyrosine at position 615 was substituted with glycine (T180V_Y615G);
the proline at position 182 was substituted with leucine and the tyrosine at position 615 was substituted with leucine (P182L_Y615L);
the arginine at position 376 was substituted with proline and the tyrosine at position 598 was substituted with asparagine (R376P_Y598N);
the glutamine at position 407 was substituted with leucine and the threonine at position 483 was substituted with lysine (Q407L_T483K);
the glutamine at position 407 was substituted with leucine and the lysine at position 487 was substituted with proline (Q407L_K487P);
the glutamine at position 407 was substituted with leucine and the tyrosine at position 615 was substituted with glycine (Q407L_Y615G);
the tyrosine at position 449 was substituted with glutamic acid and the lysine at position 487 was substituted with alanine (Y449E_K487A);
the tyrosine at position 449 was substituted with glutamic acid and the lysine at position 487 was substituted with proline (Y449E_K487P);
the threonine at position 483 was substituted with glycine and the lysine at position 487 was substituted with alanine (T483G_K487A);
the threonine at position 483 was substituted with glycine and the threonine at position 533 was substituted with glutamic acid (T483G_T533E);
the threonine at position 483 was substituted with glycine and the tyrosine at position 615 was substituted with glycine (T483G_Y615G);
the threonine at position 483 was substituted with methionine and the lysine at position 487 was substituted with alanine (T483M_K487A);
the threonine at position 483 was substituted with methionine and the lysine at position 487 was substituted with glycine (T483M_K487G);
the threonine at position 483 was substituted with methionine and the lysine at position 487 was substituted with proline (T483M_K487P);
the arginine at position 484 was substituted with histidine and the lysine at position 487 was substituted with aspartic acid (R484H_K487D);
the arginine at position 484 was substituted with lysine and the glutamic acid at position 513 was substituted with tryptophan (R484K_E513W);
the arginine at position 484 was substituted with lysine and the asparagine at position 514 was substituted with lysine (R484K_N514K);
the arginine at position 484 was substituted with lysine and the asparagine at position 514 was substituted with tryptophan (R484K N514W);
the arginine at position 484 was substituted with lysine and the asparagine at position 515 was substituted with lysine (R484K_N515K);
the arginine at position 484 was substituted with lysine and the asparagine at position 515 was substituted with tryptophan (R484K N515W);
the arginine at position 484 was substituted with lysine and the aspartic acid at position 517 was substituted with tryptophan (R484K_D517W);
the arginine at position 484 was substituted with lysine and the glycine at position 569 was substituted with lysine (R484K_G569K);
the arginine at position 484 was substituted with lysine and the glycine at position 571 was substituted with lysine (R484K_G571K);
the arginine at position 484 was substituted with lysine and the arginine at position 572 was substituted with lysine (R484K_R572K);
the arginine at position 484 was substituted with lysine and the arginine at position 572 was substituted with tryptophan (R484K_R572W);
the arginine at position 484 was substituted with lysine and the threonine at position 573 was substituted with lysine (R484K_T573K);
the arginine at position 484 was substituted with lysine and the threonine at position 573 was substituted with tryptophan (R484K_T573W);
the arginine at position 484 was substituted with lysine and the tyrosine at position 516 was substituted with lysine (R484K_Y516K);
the arginine at position 484 was substituted with tryptophan and the asparagine at position 515 was substituted with lysine (R484W_N515K);
the glycine at position 485 was substituted with aspartic acid and the lysine at position 487 was substituted with histidine (G485D_K487H);
the glycine at position 485 was substituted with lysine and the lysine at position 487 was substituted with histidine (G485K_K487H);
the glycine at position 485 was substituted with lysine and the glycine at position 569 was substituted with lysine (G485K_G569K);
the glycine at position 485 was substituted with lysine and the asparagine at position 514 was substituted with lysine (G485K_N514K);
the glycine at position 485 was substituted with lysine and the asparagine at position 515 was substituted with lysine (G485K_N515K);
the glycine at position 485 was substituted with lysine and the asparagine at position 515 was substituted with tryptophan (G485K_N515W);
the glycine at position 485 was substituted with arginine and the lysine at position 487 was substituted with aspartic acid (G485R_K487D);
the glycine at position 485 was substituted with tryptophan and the glutamic acid at position 513 was substituted with tryptophan (G485W_E513W);
the glycine at position 485 was substituted with tryptophan and the asparagine at position 515 was substituted with lysine (G485W_N515K);
the glycine at position 485 was substituted with tryptophan and the asparagine at position 515 was substituted with tryptophan (G485W_N515W);
the lysine at position 487 was substituted with alanine and the serine at position 531 was substituted with alanine (K487A_S531A);
the lysine at position 487 was substituted with alanine and the tryptophan at position 593 was substituted with phenylalanine (K487A_W593F);
the lysine at position 487 was substituted with alanine and the tyrosine at position 615 was substituted with leucine (K487A_Y615L);
the lysine at position 487 was substituted with tryptophan and the asparagine at position 514 was substituted with lysine (K487W_N514K);
the lysine at position 487 was substituted with tryptophan and the asparagine at position 514 was substituted with tryptophan (K487W N514W);
the lysine at position 487 was substituted with tryptophan and the tyrosine at position 516 was substituted with tryptophan (K487W_Y516W);
then lysine at position 487 was substituted with tryptophan and the glycine at position 569 was substituted with lysine (K487W_G569K);
the lysine at position 487 was substituted with tyrosine and the asparagine at position 515 is substituted with phenylalanine (K487Y_N515F);
the lysine at position 487 was substituted with tyrosine and the asparagine at position 515 was substituted with tryptophan (K487Y_N515W);
the valine at position 488 was substituted with lysine and the asparagine at position 515 was substituted with lysine (V488K_N515K);
the valine at position 488 was substituted with lysine and the asparagine at position 515 was substituted with tryptophan (V488K_N515W);
the valine at position 488 was substituted with tryptophan and the asparagine at position 515 was substituted with lysine (V488W_N515K);
the valine at position 488 was substituted with tryptophan and the asparagine at position 515 was substituted with tryptophan (V488W_N515W);
the valine at position 488 was substituted with tyrosine and the asparagine at position 515 was substituted with tryptophan (V488Y_N515W);
the asparagine at position 550 was substituted with serine and the tyrosine at position 598 was substituted with asparagine (N550S_Y598N);
the asparagine at position 550 was substituted with serine and the tyrosine at position 615 was substituted with glycine (N550S_Y615G);
the asparagine at position 550 was substituted with serine and the tyrosine at position 615 was substituted with leucine (N550S_Y615L); and the asparagine at position 550 was substituted with serine and the tyrosine at position 615 was substituted with proline (N550S_Y615P).
   (K) Variants meeting condition 3 and having undergone three or four amino acid substitutions (FIG. 12), in which
the arginine at position 484 was substituted with alanine, the glycine at position 485 was substituted with alanine, and the lysine at position 487 was substituted with aspartic acid (R484A_G485A_K487D);
the arginine at position 484 was substituted with aspartic acid, the glycine at position 485 was substituted with aspartic acid, and the lysine at position 487 was substituted with aspartic acid (R484D_G485D_K487D);
the glycine at position 485 was substituted with arginine, the aspartic acid at position 486 was substituted with arginine, and the lysine at position 487 was substituted with arginine (G485R_D486R_K487R);
the arginine at position 484 was substituted with alanine, the glycine at position 485 was substituted with alanine, the aspartic acid at position 486 was substituted with alanine, and the lysine at position 487 was substituted with alanine (R484A_G485A_D486A_K487A); and
the arginine at position 484 was substituted with alanine, the glycine at position 485 was substituted with alanine, the aspartic acid at position 486 was substituted with proline, and the lysine at position 487 was substituted with proline (R484A_G485A_D486P_K487P).
   (L) Variants meeting condition 4 and having undergone three or four amino acid substitutions (FIG. 12), in which
the arginine at position 484 was substituted with alanine, the glycine at position 485 was substituted with alanine, and the lysine at position 487 was substituted with aspartic acid (R484A_G485A_K487D);
the arginine at position 484 was substituted with cysteine, the aspartic acid at position 486 was substituted with histidine, and the lysine at position 487 was substituted with arginine (R484C_D486H_K487R);
the glycine at position 485 was substituted with arginine, the aspartic acid at position 486 was substituted with tryptophan, and the lysine at position 487 was substituted with tryptophan (G485R_D486W_K487W);
the glycine at position 485 was substituted with arginine, the aspartic acid at position 486 was substituted with alanine, and the lysine at position 487 was substituted with alanine (G485R_D486A_K487A);
the glycine at position 485 was substituted with arginine, the aspartic acid at position 486 was substituted with proline, and the lysine at position 487 was substituted with proline (G485R_D486P_K487P);
the arginine at position 484 was substituted with alanine, the glycine at position 485 was substituted with alanine, the aspartic acid at position 486 was substituted with alanine, and
the lysine at position 487 was substituted with alanine (R484A_G485A_D486A_K487A); the arginine at position 484 was substituted with alanine, the glycine at position 485 was substituted with alanine, the aspartic acid at position 486 was substituted with arginine, and the lysine at position 487 was substituted with arginine (R484A_G485A_D486R_K487R);
the arginine at position 484 was substituted with aspartic acid, the glycine at position 485 was substituted with aspartic acid, the aspartic acid at position 486 was substituted with arginine, and the lysine at position 487 was substituted with arginine (R484D_G485D_D486R_K487R);
the arginine at position 484 was substituted with aspartic acid, the glycine at position 485 was substituted with aspartic acid, the aspartic acid at position 486 was substituted with proline, and the lysine at position 487 was substituted with proline (R484D_G485D_D486P_K487P);
the arginine at position 484 was substituted with aspartic acid, the glycine at position 485 was substituted with aspartic acid, the aspartic acid at position 486 was substituted with alanine, and the lysine at position 487 was substituted with alanine (R484D_G485D_D486A_K487A);
the arginine at position 484 was substituted with aspartic acid, the glycine at position 485 was substituted with aspartic acid, the aspartic acid at position 486 was substituted with tryptophan, and the lysine at position 487 was substituted with tryptophan (R484D_G485D_D486W_K487W); and
the arginine at position 484 was substituted with proline, the glycine at position 485 was substituted with proline, the aspartic acid at position 486 was substituted with arginine, and the lysine at position 487 was substituted with arginine (R484P_G485P_D486R_K487R).
   (M) Insertion or deletion variants meeting condition 3 (FIG. 13), which are represented by
Δ484-487,
N514-F-F-N515,
N514-W-W-N515,
P604-W-W-Y605,
P604-A-A-A-A-Y605, and
P604-F-F-F-F-Y605.
   (N) Insertion or deletion variants meeting condition 4 (FIG. 13), which are represented by
Δ485_D486H,
Δ485-486_K487R,
Δ485-486_R484W_K487W,
Δ485-486_R484P_K487P,
R484-A-A-G485,
R484-F-F-G485,
R484-K-K-G485,
R484-P-P-G485,
R484-R-R-G485,
R484-S-S-G485,
R484-W-W-G485,
G485-S-S-S-D486,
R484-A-A-A-A-G485,
R484-D-D-D-D-G485,
R484-F-F-F-F-G485,
R484-P-P-P-P-G485,
R484-R-R-R-R-G485,
R484-S-S-S-S-G485,
R484-W-W-W-W-G485,
R484-A-A-A-A-A-A-A-A-G485,
R484-D-D-D-D-D-D-D-D-G485,
R484-F-F-F-F-F-F-F-F-G485,
R484-K-K-K-K-K-K-K-K-G485, and
R484-P-P-P-P-P-P-P-P-G485.

### INDUSTRIAL APPLICABILITY

The modified enzymes of the present invention exhibit enzymological properties different from those of the parent wild-type enzymes thereof. Taking advantage of these characteristics, it is envisaged that the modified enzymes will be used to produce various galacto-oligosaccharides separately as intended. In particular, it can be expected that the present invention is used for reproducing the profile of oligosaccharides in mother's milk, which has not been achievable using wild-type enzymes alone. In addition, the modified enzymes of the present invention will be expected to be used for production (synthesis) of oligosaccharides. Furthermore, the present invention can also be used for production and processing of pharmaceutical products and supplements for lactose-intolerant patients, production and processing of dairy products (for example, processed milk products such as lactose-reduced milk, powdered milk such as skim milk and nursing powdered milk, yogurts, and others), and production and processing of medical foods.

The present invention should not be limited in any way to the description of the above-described embodiments and examples of the invention. The present invention also includes a variety of modified embodiments within the scope that one skilled in the art could easily arrive without departing from the description of the scope of claims. The contents of articles, published patent applications, patent publications, and others that are expressly provided are incorporated in their entire content by citation.

1. A modified β-galactosidase consisting of an amino acid sequence that has at least 98% sequence identity to the amino acid sequence of SEQ ID NO. 1, 2, 3, or 4, wherein the modified β-galactosidase includes one or more amino acid substitutions selected from the group consisting of
tryptophan at position 76 (W76) is glycine;
glutamic acid at position 480 (E480) is glutamine;
threonine at position 483 (T483) is tryptophan; lysine, methionine, or arginine;
arginine at position 484 (R484) is alanine, cysteine, aspartic acid, glycine, proline, serine, tyrosine, or lysine;
glycine at position 485 (G485) is proline or threonine;
lysine at position 487 (K487) is glycine or proline;
valine at position 488 (V488) is proline;
serine at position 512 (S512) is leucine, methionine glycine or lysine;
asparagine at position 514 (N514) is aspartic acid;
asparagine at position 515 (N515) is phenylalanine, arginine, tryptophan, tyrosine, or lysine;
glutamine at position 551 (Q551) is asparagine;
glycine at position 569 (G569) is lysine, proline, or valine;
phenylalanine at position 596 (F596) is glycine;
tyrosine at position 598 (Y598) is asparagine; and
tyrosine at position 615 (Y615) is threonine,
or an amino acid(s) corresponding thereto, relative to SEQ ID NO. 1, 2, 3, or 4, wherein the modified β-galactosidase produces oligosaccharides in which the content of trisaccharides and higher saccharides is approximately equal and the content of tetrasaccharides is higher, relative to the case of the parent wild-type enzyme.

2. A polynucleotide encoding the modified β-galactosidase of claim 1.

3. A recombinant DNA comprising the polynucleotide of claim 2.

4. A microorganism carrying the recombinant DNA of claim 3.

5. An enzyme preparation comprising the modified β-galactosidase of claim 1.

6. A method for producing an oligosaccharide, the method comprising contacting the modified β-galactosidase of claim 1 with a disaccharide, an oligosaccharide, or a polysaccharide having at least one of β-1,3-bond, β-1,4-bond, β-1,6-bond, or a combination thereof.


SEQUENCE LISTING (8 sequences)

SEQ ID NO: 1 - protein, 1737 aa, Bacillus circulans
MKKAISCVFLISALILSSFQVPVQGQAMSKTTSAAGNSVSYDGERRVNFNENWRFQRETNGSIAGAQNPGFDDSSWRKLNLPHDWSIELDFNKNSLATHEGGYLDGGIGWYRKTFTIPESMKGKRISLDFDGVYMNSTTYLNGEVLGTYPFGYNAFSYDISDKLYKDGRANVLVVKVNNTQPSSRWYSGSGIYRNVYLTVTDPIHVARYGTFVTTPNLEKSIKEDRADVNIKTKISNDAAEAKQVKIKSTIYDGAGNTVQTVETEEKTAAAGTVTPFEQNTVIKQPKLWSIDKPYRYNLVTEVIVGGQTVDTYETKFGVRYFKFDENEGFSLNGEYMKLHGVSMHHDLGALGAATNARGVERQMQIMKDMGVNAIRVTHNPASPELLEAANKLGLFIIEEAFDSWAQSKKPYDYGRFFNAWAEHDIKEMVDRGKNEPAIIMWSIGNEIYDTTNAAGVETARNLVGWVKEIDTTRPTTIGEDKTRGDKVNVTPINSYIKEIFNIVDVVGLNYSENNYDGYHKQNPSWKLYGSETSSATRSRGVYTHPYQYNQSTKYADLQQSSYDNDYVGWGRTAEDAWKYDRDLKHIAGQFIWTGFDYIGEPTPYYNSYPAKSSYFGAVDTAGFPKDIFYYYQSQWKKEPMVHLLPHWNWKEGEKVRVLAYTNASKVELVLNGESLGEKNYDNKQTSWGAPYKETKDGKTYLEWAVPFKPGKLEAVAKDENGKVIARDQVVTAGEPASVRLTADRKVVKADGTDLSFITADIVDSKGIVVPDADHLITFNVTGQGELAGVDNGNASSVERYKDNKRKAFSGKALAIVQSSKLSGKITVHASVAGLSSDSTSVFTVTPADHDKKIVAGIDDVNLTVDVNEAPKLPSEIKVYYSDESAAAKNVTWDEVDPKQYSTVGEFTVEGSVEGTSLKAKAFVIVKGIVAVKPYSTATKVGVQPVLPEKATLLYSDGTTKGATVTWDEIPEDKLAKEGRFTVEGSVEGTDLKANVYVRVTNEVKSVNIMLQEQGSAYPKLEATFTNPADNLQHLNDGIKSYTNNPVNRWTNWTRTPRDAGDSITVNFGKKHVINNLDLFVFTDSGTVVPEKAEVQYWDGTAWKDVENLTQPSPYVVEKNELTFDAVATEKLKFHLTPSVKGKFLALTEAEVYADQIVMGETAKLQSITVNGKALEGFDHAKKNYELVLPYGSELPKIEAAAADNATVTILPAFSYPGTAKLFVTSEDGKVTTEYSIGVSTEEPKLVSAELSADKTNVMEDDIIDLKVIGLFESKEKIDVTDSQPTYEFDQQIIKIEGNKLYALETGNVKVKVTVTYKGVSVTTPALEFTIAKNPAPKYITSLEPVTVVVKKGEAPELPATVVAHYNRGIPRDVKVKWERINPSKYQQLGEFTVSGMVEGTDIKAQAKVIVKGAVAVEDIRMAVLLKQMPQLPGKVTVYYSDGAEEQRAVKWEEIPQEELENVGEFKVKGDVNGVKLKATATIRVTDEVGGEQNISRAKNGYEYPKAEASFTNNGPGSSDRIEAINDDVISYEANPHNRWTNWQPVPRAGDWVSITFGDYEPTEYDVDSMEIHWFADHGTSYPERFQIEYKSGDSWKEVTSLKSDPASPALGKANVYSFDRVKTSAIRVKMTAQAGKSLAITELKVFSKWPKAGTEPEVTDIKVGGKSILEDFEQKGDHYEVTIDAGDANVMPKINVKAKDQTSITIVPAVTSPSTAKVIAKSEDGKKVKVYSIHYK

SEQ ID NO: 2 - protein, 1422 aa, Bacillus circulans
MKKAISCVFLISALILSSFQVPVQGQAMSKTTSAAGNSVSYDGERRVNFNENWRFQRETNGSIAGAQNPGFDDSSWRKLNLPHDWSIELDFNKNSLATHEGGYLDGGIGWYRKTFTIPESMKGKRISLDFDGVYMNSTTYLNGEVLGTYPFGYNAFSYDISDKLYKDGRANVLVVKVNNTQPSSRWYSGSGIYRNVYLTVTDPIHVARYGTFVTTPNLEKSIKEDRADVNIKTKISNDAAEAKQVKIKSTIYDGAGNTVQTVETEEKTAAAGTVTPFEQNTVIKQPKLWSIDKPYRYNLVTEVIVGGQTVDTYETKFGVRYFKFDENEGFSLNGEYMKLHGVSMHHDLGALGAATNARGVERQMQIMKDMGVNAIRVTHNPASPELLEAANKLGLFIIEEAFDSWAQSKKPYDYGRFFNAWAEHDIKEMVDRGKNEPAIIMWSIGNEIYDTTNAAGVETARNLVGWVKEIDTTRPTTIGEDKTRGDKVNVTPINSYIKEIFNIVDVVGLNYSENNYDGYHKQNPSWKLYGSETSSATRSRGVYTHPYQYNQSTKYADLQQSSYDNDYVGWGRTAEDAWKYDRDLKHIAGQFIWTGFDYIGEPTPYYNSYPAKSSYFGAVDTAGFPKDIFYYYQSQWKKEPMVHLLPHWNWKEGEKVRVLAYTNASKVELVLNGESLGEKNYDNKQTSWGAPYKETKDGKTYLEWAVPFKPGKLEAVAKDENGKVIARDQVVTAGEPASVRLTADRKVVKADGTDLSFITADIVDSKGIVVPDADHLITFNVTGQGELAGVDNGNASSVERYKDNKRKAFSGKALAIVQSSKLSGKITVHASVAGLSSDSTSVFTVTPADHDKKIVAGIDDVNLTVDVNEAPKLPSEIKVYYSDESAAAKNVTWDEVDPKQYSTVGEFTVEGSVEGTSLKAKAFVIVKGIVAVKPYSTATKVGVQPVLPEKATLLYSDGTTKGATVTWDEIPEDKLAKEGRFTVEGSVEGTDLKANVYVRVTNEVKSVNIMLQEQGSAYPKLEATFTNPADNLQHLNDGIKSYTNNPVNRWTNWTRTPRDAGDSITVNFGKKHVINNLDLFVFTDSGTVVPEKAEVQYWDGTAWKDVENLTQPSPYVVEKNELTFDAVATEKLKFHLTPSVKGKFLALTEAEVYADQIVMGETAKLQSITVNGKALEGFDHAKKNYELVLPYGSELPKIEAAAADNATVTILPAFSYPGTAKLFVTSEDGKVTTEYSIGVSTEEPKLVSAELSADKTNVMEDDIIDLKVIGLFESKEKIDVTDSQPTYEFDQQIIKIEGNKLYALETGNVKVKVTVTYKGVSVTTPALEFTIAKNPAPKYITSLEPVTVVVKKGEAPELPATVVAHYNRGIPRDVKVKWERINPSKYQQLGEFTVSGMVEGTDIKAQAKVIVKGAVAVEDIRM

SEQ ID NO: 3 - protein, 1249 aa, Bacillus circulans
MKKAISCVFLISALILSSFQVPVQGQAMSKTTSAAGNSVSYDGERRVNFNENWRFQRETNGSIAGAQNPGFDDSSWRKLNLPHDWSIELDFNKNSLATHEGGYLDGGIGWYRKTFTIPESMKGKRISLDFDGVYMNSTTYLNGEVLGTYPFGYNAFSYDISDKLYKDGRANVLVVKVNNTQPSSRWYSGSGIYRNVYLTVTDPIHVARYGTFVTTPNLEKSIKEDRADVNIKTKISNDAAEAKQVKIKSTIYDGAGNTVQTVETEEKTAAAGTVTPFEQNTVIKQPKLWSIDKPYRYNLVTEVIVGGQTVDTYETKFGVRYFKFDENEGFSLNGEYMKLHGVSMHHDLGALGAATNARGVERQMQIMKDMGVNAIRVTHNPASPELLEAANKLGLFIIEEAFDSWAQSKKPYDYGRFFNAWAEHDIKEMVDRGKNEPAIIMWSIGNEIYDTTNAAGVETARNLVGWVKEIDTTRPTTIGEDKTRGDKVNVTPINSYIKEIFNIVDVVGLNYSENNYDGYHKQNPSWKLYGSETSSATRSRGVYTHPYQYNQSTKYADLQQSSYDNDYVGWGRTAEDAWKYDRDLKHIAGQFIWTGFDYIGEPTPYYNSYPAKSSYFGAVDTAGFPKDIFYYYQSQWKKEPMVHLLPHWNWKEGEKVRVLAYTNASKVELVLNGESLGEKNYDNKQTSWGAPYKETKDGKTYLEWAVPFKPGKLEAVAKDENGKVIARDQVVTAGEPASVRLTADRKVVKADGTDLSFITADIVDSKGIVVPDADHLITFNVTGQGELAGVDNGNASSVERYKDNKRKAFSGKALAIVQSSKLSGKITVHASVAGLSSDSTSVFTVTPADHDKKIVAGIDDVNLTVDVNEAPKLPSEIKVYYSDESAAAKNVTWDEVDPKQYSTVGEFTVEGSVEGTSLKAKAFVIVKGIVAVKPYSTATKVGVQPVLPEKATLLYSDGTTKGATVTWDEIPEDKLAKEGRFTVEGSVEGTDLKANVYVRVTNEVKSVNIMLQEQGSAYPKLEATFTNPADNLQHLNDGIKSYTNNPVNRWTNWTRTPRDAGDSITVNFGKKHVINNLDLFVFTDSGTVVPEKAEVQYWDGTAWKDVENLTQPSPYVVEKNELTFDAVATEKLKFHLTPSVKGKFLALTEAEVYADQIVMGETAKLQSITVNGKALEGFDHAKKNYELVLPYGSELPKIEAAAADNATVTILPAFSYPGTAKLFVTSEDGKVTTEYSIGVSTEEPKLVSA

SEQ ID NO: 4 - protein, 847 aa, Bacillus circulans
MKKAISCVFLISALILSSFQVPVQGQAMSKTTSAAGNSVSYDGERRVNFNENWRFQRETNGSIAGAQNPGFDDSSWRKLNLPHDWSIELDFNKNSLATHEGGYLDGGIGWYRKTFTIPESMKGKRISLDFDGVYMNSTTYLNGEVLGTYPFGYNAFSYDISDKLYKDGRANVLVVKVNNTQPSSRWYSGSGIYRNVYLTVTDPIHVARYGTFVTTPNLEKSIKEDRADVNIKTKISNDAAEAKQVKIKSTIYDGAGNTVQTVETEEKTAAAGTVTPFEQNTVIKQPKLWSIDKPYRYNLVTEVIVGGQTVDTYETKFGVRYFKFDENEGFSLNGEYMKLHGVSMHHDLGALGAATNARGVERQMQIMKDMGVNAIRVTHNPASPELLEAANKLGLFIIEEAFDSWAQSKKPYDYGRFFNAWAEHDIKEMVDRGKNEPAIIMWSIGNEIYDTTNAAGVETARNLVGWVKEIDTTRPTTIGEDKTRGDKVNVTPINSYIKEIFNIVDVVGLNYSENNYDGYHKQNPSWKLYGSETSSATRSRGVYTHPYQYNQSTKYADLQQSSYDNDYVGWGRTAEDAWKYDRDLKHIAGQFIWTGFDYIGEPTPYYNSYPAKSSYFGAVDTAGFPKDIFYYYQSQWKKEPMVHLLPHWNWKEGEKVRVLAYTNASKVELVLNGESLGEKNYDNKQTSWGAPYKETKDGKTYLEWAVPFKPGKLEAVAKDENGKVIARDQVVTAGEPASVRLTADRKVVKADGTDLSFITADIVDSKGIVVPDADHLITFNVTGQGELAGVDNGNASSVERYKDNKRKAFSGKALAIVQSSKLSGKITVHASVAGLSSDSTSVFTVTP

SEQ ID NO: 5 - DNA, 5214 bp, Bacillus circulans
  source 1..5214: mol_type = unassigned DNA
gtgaaaaaagcgattagctgcgtttttttaatttcagcactgattctatcaagctttcaagtccctgtacagggacaagccatgtcaaaaacgacatcggcagcaggaaacagtgtgagctatgatggagagagacgagtgaattttaacgagaattggcgatttcaacgagaaaccaatggaagtattgccggagcacagaatcctggctttgacgattcctcctggcggaaattaaatctgccgcatgactggagtattgaattagattttaataaaaattctcttgccacacatgaaggcggttatttggacggcggaatcggctggtaccgaaaaacctttacaatcccggaatcgatgaagggaaaacgaatttcgcttgattttgatggcgtttacatgaacagcaccacctatctaaacggggaagtgctcgggacctatccgtttggttataatgccttttcctatgatatttccgacaaactttataaagatggcagggcgaatgtccttgttgtcaaagtcaataacacccagccgagcagccgctggtattcggggagcgggatctaccggaatgtctatctcactgtgaccgatcccatccatgtggctcgctacggaacatttgtgacaacacccaatttagagaaatcgataaaagaagacagggctgatgtgaacatcaagacgaaaatcagtaacgatgctgctgaggcgaaacaggtaaagattaaatcaaccatctacgatggggctgggaacaccgtacagacagtggaaacggaggaaaaaacagctgccgccggcacggtgactccgttcgaacaaaacacagtcatcaagcagccgaagctttggagcattgacaagccttatcgatataaccttgttacagaagtcatcgttggcgggcaaacggtggatacgtatgaaacaaaatttggtgtcaggtatttcaaatttgatgaaaacgaaggcttttccttaaatggagagtatatgaagctgcacggcgtttcgatgcaccatgatttaggggcgcttggggcggcaacgaatgcacgcggcgtggaaagacaaatgcagattatgaaggatatgggggtcaatgccatcagggttacccacaacccggcatcaccggaactgctggaggcagctaataaattagggctattcatcatcgaggaggcatttgacagctgggcccagtcaaagaaaccctatgactatggccgttttttcaatgcatgggctgagcacgacattaaggaaatggtcgatcggggcaaaaacgaaccagctattatcatgtggtcgatcggaaatgaaatatatgatacgaccaatgccgctggtgtggaaacagcacgaaatttagtgggttgggtaaaagaaattgacaccacaaggccgacaacgatcggcgaggataaaacccgcggagacaaagtaaatgttacacctatcaacagctacatcaaggagatttttaatattgtcgatgtggtcggactgaactacagcgagaacaactatgatggctaccacaagcagaatccgtcatggaagctgtacggctcggagacgtcctcggcaacccgttcgcgtggtgtctacacgcatccgtaccagtataaccaaagcacaaagtatgctgatttacagcaatcctcttatgacaatgactatgtcggctggggacgaactgcagaagatgcatggaaatatgaccgcgacctgaagcatattgcagggcaatttatctggaccggctttgattatattggcgagccgacgccatattataattcctatcctgcaaaaagctcctattttggtgctgtggatacggctggttttccaaaggatattttctactattaccaaagccaatggaaaaaggagcctatggtccacctgctgccgcattggaactggaaggaaggggaaaaggtccgcgtcttagcttataccaatgcaagtaaggttgaacttgttctaaatggtgaatcgttaggggagaagaactatgacaacaaacaaacctcctggggagcaccatacaaagaaacaaaggatggaaaaacctatttggagtgggccgtaccatttaaaccgggcaaattagaagccgtcgccaaggatgaaaacggcaaagtgatcgcccgcgatcaggtagtgaccgctggtgagccagcctctgtcagattaacggctgatcgtaaggtggtcaaggcggacggtacggatctgtcgtttattacagcagacattgttgatagtaaagggattgttgtcccggatgccgatcatctgattacatttaacgtaacgggccaaggggaattggccggggttgataacggaaacgcgtccagtgtggagcgttacaaggacaacaagcgcaaggctttcagcgggaaagcattggcgattgttcaatcaagtaagctttctggaaaaattacggtccatgcgtcagtggcagggctttcgagcgattccacgagcgtatttacggtaacgccagctgaccatgacaaaaagattgtagctgggattgatgatgttaaccttactgtcgatgtcaatgaagcaccaaagcttccttcagaaatcaaggtttattacagtgatgagagtgcagctgcgaagaatgtgacttgggatgaggtggatccaaagcagtacagcactgttggtgaattcacagtggaaggcagtgtcgagggaacttcgctgaaggcaaaggcatttgttattgtcaaaggaattgtcgccgtcaagccttattcaacggcaacaaaggttggtgtacagccggtgctgcctgaaaaagcaacccttctttacagtgatggaacaaccaagggagcaactgtcacgtgggatgagatccctgaggacaagctggcaaaagagggccggtttaccgtcgagggcagtgtggagggaacagacctcaaggctaatgtctatgtcagggtgacaaatgaagtaaaatcagtgaatattatgcttcaggagcagggttcagcttatccaaagctcgaagctacttttaccaatccagctgacaatcttcagcatttgaacgatggcatcaagagctataccaataacccggtcaaccgctggacgaactggacaagaacaccgcgtgatgctggtgactcgattacagttaattttggcaagaagcatgtgattaataatctagatttatttgtttttaccgacagcggcacggtggttccagaaaaggcagaggtccaatattgggatggaacggcgtggaaggatgtcgaaaatctaacacagccatcgccatatgtggtagagaaaaatgaacttacatttgatgcggtcgcgacagaaaagctgaaattccatttgacaccatctgtgaaagggaaattcctagctctaacggaagcagaggtgtacgccgatcagattgtgatgggtgaaacagcaaaacttcaaagtattacggtgaatgggaaagcattagaaggctttgatcacgctaaaaagaattatgaacttgtacttccatatggaagcgagcttcctaagattgaggcggctgctgccgacaatgcaactgtcaccattttaccggcattctcctatccgggaacagcaaaactatttgtcacttcagaggatgggaaggtaactactgagtacagtattggtgtttctacagaagagccaaagctcgtctccgcagagttatccgcggacaagacgaatgtcatggaggacgatatcatcgatctgaaggtaattggtctcttcgaaagcaaggaaaagattgatgtgaccgacagccagccgacatatgaatttgaccagcagattattaaaattgaaggcaataagctgtatgcgctggaaacaggaaatgtcaaggtgaaagtgacggtgacatataagggtgtgagtgtcacaacacctgcgcttgagtttacgatcgcgaaaaaccctgctccaaaatacattacgagcttagagcctgtcacggttgttgttaaaaaaggagaagcgccggagcttccagcaacggttgtggcccattataaccgaggaatcccgcgggatgttaaggtgaagtgggaaagaatcaatccgtctaagtaccagcagctaggcgagtttaccgtatctggcatggtggaagggaccgatataaaagcccaagcaaaagtgattgtaaaaggggctgttgcggtcgaggatattagaatggctgtgctgttaaagcaaatgccacagctgccgggcaaggttacagtctattatagtgacggagcggaagaacaaagagcggtcaagtgggaggaaatcccgcaggaggaactcgagaatgtcggtgaatttaaggttaaaggtgatgttaatggagtgaagctgaaagcaacagccactattcgagtaaccgatgaagtcggcggcgagcagaatatcagccgggctaaaaatggttatgaatacccgaaggctgaagcttcctttaccaacaatggccctggatcaagcgatcgaatcgaggccatcaatgatgacgtgatctcctacgaggctaatccgcataatcgctggacgaattggcagccggtaccgcgtgcaggggactgggtttctatcacctttggagactatgagcctacggaatatgatgttgatagcatggagatccactggttcgcggatcatgggacctcgtatccagagcgtttccaaatcgaatataaatccggtgatagctggaaggaagtcaccagcctgaaaagcgatccagcctctccggccttgggtaaggcaaatgtctatagctttgatcgagtaaaaacatcggctatacgagtgaaaatgacagcacaagccggcaaaagcttagccattaccgagctgaaagtattttcaaaatggccaaaggcaggtaccgaaccagaggtgaccgatattaaggtcggaggaaaatcgattctggaggactttgaacaaaaaggcgatcactatgaagtaacgattgatgcaggagatgcgaatgtaatgccgaaaatcaatgtaaaggctaaggaccagacgagtattacgattgtgccagcagttacctctccatccacggcaaaggtaattgctaaatccgaggatggcaagaaagtgaaggtctatagcattcactataaataa

SEQ ID NO: 6 - DNA, 4266 bp, Bacillus circulans
  source 1..4266: mol_type = unassigned DNA
gtgaaaaaagcgattagctgcgtttttttaatttcagcactgattctatcaagctttcaagtccctgtacagggacaagccatgtcaaaaacgacatcggcagcaggaaacagtgtgagctatgatggagagagacgagtgaattttaacgagaattggcgatttcaacgagaaaccaatggaagtattgccggagcacagaatcctggctttgacgattcctcctggcggaaattaaatctgccgcatgactggagtattgaattagattttaataaaaattctcttgccacacatgaaggcggttatttggacggcggaatcggctggtaccgaaaaacctttacaatcccggaatcgatgaagggaaaacgaatttcgcttgattttgatggcgtttacatgaacagcaccacctatctaaacggggaagtgctcgggacctatccgtttggttataatgccttttcctatgatatttccgacaaactttataaagatggcagggcgaatgtccttgttgtcaaagtcaataacacccagccgagcagccgctggtattcggggagcgggatctaccggaatgtctatctcactgtgaccgatcccatccatgtggctcgctacggaacatttgtgacaacacccaatttagagaaatcgataaaagaagacagggctgatgtgaacatcaagacgaaaatcagtaacgatgctgctgaggcgaaacaggtaaagattaaatcaaccatctacgatggggctgggaacaccgtacagacagtggaaacggaggaaaaaacagctgccgccggcacggtgactccgttcgaacaaaacacagtcatcaagcagccgaagctttggagcattgacaagccttatcgatataaccttgttacagaagtcatcgttggcgggcaaacggtggatacgtatgaaacaaaatttggtgtcaggtatttcaaatttgatgaaaacgaaggcttttccttaaatggagagtatatgaagctgcacggcgtttcgatgcaccatgatttaggggcgcttggggcggcaacgaatgcacgcggcgtggaaagacaaatgcagattatgaaggatatgggggtcaatgccatcagggttacccacaacccggcatcaccggaactgctggaggcagctaataaattagggctattcatcatcgaggaggcatttgacagctgggcccagtcaaagaaaccctatgactatggccgttttttcaatgcatgggctgagcacgacattaaggaaatggtcgatcggggcaaaaacgaaccagctattatcatgtggtcgatcggaaatgaaatatatgatacgaccaatgccgctggtgtggaaacagcacgaaatttagtgggttgggtaaaagaaattgacaccacaaggccgacaacgatcggcgaggataaaacccgcggagacaaagtaaatgttacacctatcaacagctacatcaaggagatttttaatattgtcgatgtggtcggactgaactacagcgagaacaactatgatggctaccacaagcagaatccgtcatggaagctgtacggctcggagacgtcctcggcaacccgttcgcgtggtgtctacacgcatccgtaccagtataaccaaagcacaaagtatgctgatttacagcaatcctcttatgacaatgactatgtcggctggggacgaactgcagaagatgcatggaaatatgaccgcgacctgaagcatattgcagggcaatttatctggaccggctttgattatattggcgagccgacgccatattataattcctatcctgcaaaaagctcctattttggtgctgtggatacggctggttttccaaaggatattttctactattaccaaagccaatggaaaaaggagcctatggtccacctgctgccgcattggaactggaaggaaggggaaaaggtccgcgtcttagcttataccaatgcaagtaaggttgaacttgttctaaatggtgaatcgttaggggagaagaactatgacaacaaacaaacctcctggggagcaccatacaaagaaacaaaggatggaaaaacctatttggagtgggccgtaccatttaaaccgggcaaattagaagccgtcgccaaggatgaaaacggcaaagtgatcgcccgcgatcaggtagtgaccgctggtgagccagcctctgtcagattaacggctgatcgtaaggtggtcaaggcggacggtacggatctgtcgtttattacagcagacattgttgatagtaaagggattgttgtcccggatgccgatcatctgattacatttaacgtaacgggccaaggggaattggccggggttgataacggaaacgcgtccagtgtggagcgttacaaggacaacaagcgcaaggctttcagcgggaaagcattggcgattgttcaatcaagtaagctttctggaaaaattacggtccatgcgtcagtggcagggctttcgagcgattccacgagcgtatttacggtaacgccagctgaccatgacaaaaagattgtagctgggattgatgatgttaaccttactgtcgatgtcaatgaagcaccaaagcttccttcagaaatcaaggtttattacagtgatgagagtgcagctgcgaagaatgtgacttgggatgaggtggatccaaagcagtacagcactgttggtgaattcacagtggaaggcagtgtcgagggaacttcgctgaaggcaaaggcatttgttattgtcaaaggaattgtcgccgtcaagccttattcaacggcaacaaaggttggtgtacagccggtgctgcctgaaaaagcaacccttctttacagtgatggaacaaccaagggagcaactgtcacgtgggatgagatccctgaggacaagctggcaaaagagggccggtttaccgtcgagggcagtgtggagggaacagacctcaaggctaatgtctatgtcagggtgacaaatgaagtaaaatcagtgaatattatgcttcaggagcagggttcagcttatccaaagctcgaagctacttttaccaatccagctgacaatcttcagcatttgaacgatggcatcaagagctataccaataacccggtcaaccgctggacgaactggacaagaacaccgcgtgatgctggtgactcgattacagttaattttggcaagaagcatgtgattaataatctagatttatttgtttttaccgacagcggcacggtggttccagaaaaggcagaggtccaatattgggatggaacggcgtggaaggatgtcgaaaatctaacacagccatcgccatatgtggtagagaaaaatgaacttacatttgatgcggtcgcgacagaaaagctgaaattccatttgacaccatctgtgaaagggaaattcctagctctaacggaagcagaggtgtacgccgatcagattgtgatgggtgaaacagcaaaacttcaaagtattacggtgaatgggaaagcattagaaggctttgatcacgctaaaaagaattatgaacttgtacttccatatggaagcgagcttcctaagattgaggcggctgctgccgacaatgcaactgtcaccattttaccggcattctcctatccgggaacagcaaaactatttgtcacttcagaggatgggaaggtaactactgagtacagtattggtgtttctacagaagagccaaagctcgtctccgcagagttatccgcggacaagacgaatgtcatggaggacgatatcatcgatctgaaggtaattggtctcttcgaaagcaaggaaaagattgatgtgaccgacagccagccgacatatgaatttgaccagcagattattaaaattgaaggcaataagctgtatgcgctggaaacaggaaatgtcaaggtgaaagtgacggtgacatataagggtgtgagtgtcacaacacctgcgcttgagtttacgatcgcgaaaaaccctgctccaaaatacattacgagcttagagcctgtcacggttgttgttaaaaaaggagaagcgccggagcttccagcaacggttgtggcccattataaccgaggaatcccgcgggatgttaaggtgaagtgggaaagaatcaatccgtctaagtaccagcagctaggcgagtttaccgtatctggcatggtggaagggaccgatataaaagcccaagcaaaagtgattgtaaaaggggctgttgcggtcgaggatattagaatg

SEQ ID NO: 7 - DNA, 3747 bp, Bacillus circulans
  source 1..3747: mol_type = unassigned DNA
gtgaaaaaagcgattagctgcgtttttttaatttcagcactgattctatcaagctttcaagtccctgtacagggacaagccatgtcaaaaacgacatcggcagcaggaaacagtgtgagctatgatggagagagacgagtgaattttaacgagaattggcgatttcaacgagaaaccaatggaagtattgccggagcacagaatcctggctttgacgattcctcctggcggaaattaaatctgccgcatgactggagtattgaattagattttaataaaaattctcttgccacacatgaaggcggttatttggacggcggaatcggctggtaccgaaaaacctttacaatcccggaatcgatgaagggaaaacgaatttcgcttgattttgatggcgtttacatgaacagcaccacctatctaaacggggaagtgctcgggacctatccgtttggttataatgccttttcctatgatatttccgacaaactttataaagatggcagggcgaatgtccttgttgtcaaagtcaataacacccagccgagcagccgctggtattcggggagcgggatctaccggaatgtctatctcactgtgaccgatcccatccatgtggctcgctacggaacatttgtgacaacacccaatttagagaaatcgataaaagaagacagggctgatgtgaacatcaagacgaaaatcagtaacgatgctgctgaggcgaaacaggtaaagattaaatcaaccatctacgatggggctgggaacaccgtacagacagtggaaacggaggaaaaaacagctgccgccggcacggtgactccgttcgaacaaaacacagtcatcaagcagccgaagctttggagcattgacaagccttatcgatataaccttgttacagaagtcatcgttggcgggcaaacggtggatacgtatgaaacaaaatttggtgtcaggtatttcaaatttgatgaaaacgaaggcttttccttaaatggagagtatatgaagctgcacggcgtttcgatgcaccatgatttaggggcgcttggggcggcaacgaatgcacgcggcgtggaaagacaaatgcagattatgaaggatatgggggtcaatgccatcagggttacccacaacccggcatcaccggaactgctggaggcagctaataaattagggctattcatcatcgaggaggcatttgacagctgggcccagtcaaagaaaccctatgactatggccgttttttcaatgcatgggctgagcacgacattaaggaaatggtcgatcggggcaaaaacgaaccagctattatcatgtggtcgatcggaaatgaaatatatgatacgaccaatgccgctggtgtggaaacagcacgaaatttagtgggttgggtaaaagaaattgacaccacaaggccgacaacgatcggcgaggataaaacccgcggagacaaagtaaatgttacacctatcaacagctacatcaaggagatttttaatattgtcgatgtggtcggactgaactacagcgagaacaactatgatggctaccacaagcagaatccgtcatggaagctgtacggctcggagacgtcctcggcaacccgttcgcgtggtgtctacacgcatccgtaccagtataaccaaagcacaaagtatgctgatttacagcaatcctcttatgacaatgactatgtcggctggggacgaactgcagaagatgcatggaaatatgaccgcgacctgaagcatattgcagggcaatttatctggaccggctttgattatattggcgagccgacgccatattataattcctatcctgcaaaaagctcctattttggtgctgtggatacggctggttttccaaaggatattttctactattaccaaagccaatggaaaaaggagcctatggtccacctgctgccgcattggaactggaaggaaggggaaaaggtccgcgtcttagcttataccaatgcaagtaaggttgaacttgttctaaatggtgaatcgttaggggagaagaactatgacaacaaacaaacctcctggggagcaccatacaaagaaacaaaggatggaaaaacctatttggagtgggccgtaccatttaaaccgggcaaattagaagccgtcgccaaggatgaaaacggcaaagtgatcgcccgcgatcaggtagtgaccgctggtgagccagcctctgtcagattaacggctgatcgtaaggtggtcaaggcggacggtacggatctgtcgtttattacagcagacattgttgatagtaaagggattgttgtcccggatgccgatcatctgattacatttaacgtaacgggccaaggggaattggccggggttgataacggaaacgcgtccagtgtggagcgttacaaggacaacaagcgcaaggctttcagcgggaaagcattggcgattgttcaatcaagtaagctttctggaaaaattacggtccatgcgtcagtggcagggctttcgagcgattccacgagcgtatttacggtaacgccagctgaccatgacaaaaagattgtagctgggattgatgatgttaaccttactgtcgatgtcaatgaagcaccaaagcttccttcagaaatcaaggtttattacagtgatgagagtgcagctgcgaagaatgtgacttgggatgaggtggatccaaagcagtacagcactgttggtgaattcacagtggaaggcagtgtcgagggaacttcgctgaaggcaaaggcatttgttattgtcaaaggaattgtcgccgtcaagccttattcaacggcaacaaaggttggtgtacagccggtgctgcctgaaaaagcaacccttctttacagtgatggaacaaccaagggagcaactgtcacgtgggatgagatccctgaggacaagctggcaaaagagggccggtttaccgtcgagggcagtgtggagggaacagacctcaaggctaatgtctatgtcagggtgacaaatgaagtaaaatcagtgaatattatgcttcaggagcagggttcagcttatccaaagctcgaagctacttttaccaatccagctgacaatcttcagcatttgaacgatggcatcaagagctataccaataacccggtcaaccgctggacgaactggacaagaacaccgcgtgatgctggtgactcgattacagttaattttggcaagaagcatgtgattaataatctagatttatttgtttttaccgacagcggcacggtggttccagaaaaggcagaggtccaatattgggatggaacggcgtggaaggatgtcgaaaatctaacacagccatcgccatatgtggtagagaaaaatgaacttacatttgatgcggtcgcgacagaaaagctgaaattccatttgacaccatctgtgaaagggaaattcctagctctaacggaagcagaggtgtacgccgatcagattgtgatgggtgaaacagcaaaacttcaaagtattacggtgaatgggaaagcattagaaggctttgatcacgctaaaaagaattatgaacttgtacttccatatggaagcgagcttcctaagattgaggcggctgctgccgacaatgcaactgtcaccattttaccggcattctcctatccgggaacagcaaaactatttgtcacttcagaggatgggaaggtaactactgagtacagtattggtgtttctacagaagagccaaagctcgtctccgca

SEQ ID NO: 8 - DNA, 2541 bp, Bacillus circulans
  source 1..2541: mol_type = unassigned DNA
gtgaaaaaagcgattagctgcgtttttttaatttcagcactgattctatcaagctttcaagtccctgtacagggacaagccatgtcaaaaacgacatcggcagcaggaaacagtgtgagctatgatggagagagacgagtgaattttaacgagaattggcgatttcaacgagaaaccaatggaagtattgccggagcacagaatcctggctttgacgattcctcctggcggaaattaaatctgccgcatgactggagtattgaattagattttaataaaaattctcttgccacacatgaaggcggttatttggacggcggaatcggctggtaccgaaaaacctttacaatcccggaatcgatgaagggaaaacgaatttcgcttgattttgatggcgtttacatgaacagcaccacctatctaaacggggaagtgctcgggacctatccgtttggttataatgccttttcctatgatatttccgacaaactttataaagatggcagggcgaatgtccttgttgtcaaagtcaataacacccagccgagcagccgctggtattcggggagcgggatctaccggaatgtctatctcactgtgaccgatcccatccatgtggctcgctacggaacatttgtgacaacacccaatttagagaaatcgataaaagaagacagggctgatgtgaacatcaagacgaaaatcagtaacgatgctgctgaggcgaaacaggtaaagattaaatcaaccatctacgatggggctgggaacaccgtacagacagtggaaacggaggaaaaaacagctgccgccggcacggtgactccgttcgaacaaaacacagtcatcaagcagccgaagctttggagcattgacaagccttatcgatataaccttgttacagaagtcatcgttggcgggcaaacggtggatacgtatgaaacaaaatttggtgtcaggtatttcaaatttgatgaaaacgaaggcttttccttaaatggagagtatatgaagctgcacggcgtttcgatgcaccatgatttaggggcgcttggggcggcaacgaatgcacgcggcgtggaaagacaaatgcagattatgaaggatatgggggtcaatgccatcagggttacccacaacccggcatcaccggaactgctggaggcagctaataaattagggctattcatcatcgaggaggcatttgacagctgggcccagtcaaagaaaccctatgactatggccgttttttcaatgcatgggctgagcacgacattaaggaaatggtcgatcggggcaaaaacgaaccagctattatcatgtggtcgatcggaaatgaaatatatgatacgaccaatgccgctggtgtggaaacagcacgaaatttagtgggttgggtaaaagaaattgacaccacaaggccgacaacgatcggcgaggataaaacccgcggagacaaagtaaatgttacacctatcaacagctacatcaaggagatttttaatattgtcgatgtggtcggactgaactacagcgagaacaactatgatggctaccacaagcagaatccgtcatggaagctgtacggctcggagacgtcctcggcaacccgttcgcgtggtgtctacacgcatccgtaccagtataaccaaagcacaaagtatgctgatttacagcaatcctcttatgacaatgactatgtcggctggggacgaactgcagaagatgcatggaaatatgaccgcgacctgaagcatattgcagggcaatttatctggaccggctttgattatattggcgagccgacgccatattataattcctatcctgcaaaaagctcctattttggtgctgtggatacggctggttttccaaaggatattttctactattaccaaagccaatggaaaaaggagcctatggtccacctgctgccgcattggaactggaaggaaggggaaaaggtccgcgtcttagcttataccaatgcaagtaaggttgaacttgttctaaatggtgaatcgttaggggagaagaactatgacaacaaacaaacctcctggggagcaccatacaaagaaacaaaggatggaaaaacctatttggagtgggccgtaccatttaaaccgggcaaattagaagccgtcgccaaggatgaaaacggcaaagtgatcgcccgcgatcaggtagtgaccgctggtgagccagcctctgtcagattaacggctgatcgtaaggtggtcaaggcggacggtacggatctgtcgtttattacagcagacattgttgatagtaaagggattgttgtcccggatgccgatcatctgattacatttaacgtaacgggccaaggggaattggccggggttgataacggaaacgcgtccagtgtggagcgttacaaggacaacaagcgcaaggctttcagcgggaaagcattggcgattgttcaatcaagtaagctttctggaaaaattacggtccatgcgtcagtggcagggctttcgagcgattccacgagcgtatttacggtaacgcca